(19) United States
(12) Patent Application Publication
(10) Pub. No.: US 2023/0146776 A1
(43) Pub. Date: May 11, 2023

(54) COMPOUND FOR ORGANIC ELECTRIC DEVICE, ORGANIC ELECTRIC DEVICE USING SAME, AND ELECTRONIC DEVICE THEREOF

(71) Applicant: DUK SAN NEOLUX CO., LTD., Cheonan-si, Chungcheongnam-do (KR)

(72) Inventors: Seok Won CHO, Cheonan-si, Chungcheongnam-do (KR); Jin Ho YUN, Cheonan-si, Chungcheongnam-do (KR); Yong Wook PARK, Cheonan-si, Chungcheongnam-do (KR); Young Hoon KANG, Cheonan-si, Chungcheongnam-do (KR); Min Ji JO, Cheonan-si, Chungcheongnam-do (KR)

(73) Assignee: DUK SAN NEOLUX CO., LTD., Cheonan-si, Chungcheongnam-do (KR)

(21) Appl. No.: 17/995,023

(22) PCT Filed: Mar. 4, 2021
(86) PCT No.: PCT/KR2021/002703

§ 371 Date: Sep. 29, 2022

(30) Foreign Application Priority Data
Mar. 31, 2020 (KR) 10-2020-0038749

(51) Int. Cl. H10K 85/60; C07C 211/61; C09K 11/06; C07D 307/91; C07D 215/40; C07D 319/24

(52) U.S. Cl. CPC H01L 51/006; C07C 211/61; C09K 11/06; C07D 307/91; H01L 51/0061; C07D 215/40; C07D 319/24; H01L 51/5012

(57) ABSTRACT

Provided is a compound for an organic electric device, an organic electric device using the same, and an electronic device including the organic electric device. Further provided is an organic electric device having high luminous efficiency, low driving voltage, and high heat resistance can be provided, and color purity and lifespan of the organic electric device can be improved.

### TECHNICAL FIELD

The present disclosure relates to a compound for an organic electric device, an organic electric device using the same, and an electronic device thereof.

### BACKGROUND ART

In general, an organic light emitting phenomenon refers to a phenomenon in which electric energy is converted into light energy using an organic material. An organic electric device using the organic light emitting phenomenon usually has a structure including an anode, a cathode, and an organic material layer therebetween. Here, the organic material layer is often formed of a multi-layered structure composed of different materials in order to increase the efficiency and stability of the organic electric device, and may be formed as, for example, a hole injection layer, a hole transport layer, a light emitting layer, an electron transport layer, and an electron injection layer.

A material used as an organic material layer in an organic electric device may be classified into a light emitting material and a charge transport material, e.g., a hole injection material, a hole transport material, an electron transport material, and an electron injection material depending on its functions. In addition, the light emitting material may be classified into a high molecular type and a low molecular type depending on the molecular weight, and may be classified into a fluorescent material derived from a singlet excited state of electrons and a phosphorescent material derived from a triplet excited state of electrons depending on the light emission mechanism. Further, the light emitting material may be divided into blue, green, and red light emitting materials depending on the emission color, and yellow and orange light emitting materials necessary for realizing a better natural color.

Meanwhile, since, when only one material is used as a light emitting material, there are problems in that the maximum emission wavelength is shifted to a long wavelength and the color purity is lowered due to intermolecular interaction, or the efficiency of the device is reduced due to luminescence attenuation effect, a host/dopant system may be used as a light emitting material in order to increase luminous efficiency through color purity increase and energy transfer. The principle is that when a small amount of a dopant having a smaller energy band gap than that of the host forming the light emitting layer is mixed in the light emitting layer, excitons generated in the light emitting layer are transported to the dopant to emit light with high efficiency. At this time, since the wavelength of the host moves to the wavelength band of the dopant, light having a desired wavelength may be obtained depending on the type of a dopant used.

Currently, the portable display market is a large-area display, and the size thereof is in a trend of increasing, and thus, more power consumption than the power consumption required by the existing portable display is required. Therefore, power consumption has become an important factor from the standpoint of a portable display having a limited power supply called a battery, and efficiency and lifespan problems are also important factors that should be solved.

Efficiency, lifespan, and driving voltage are related to each other, and when the efficiency is increased, the driving voltage is relatively decreased. As the driving voltage is lowered, crystallization of the organic material due to Joule heating generated during driving decreases, and as a result, the lifespan tends to increase. However, the efficiency cannot be maximized by simply improving the organic material layer. This is because, when the energy level and T1 value between respective organic material layers, and the intrinsic properties (mobility, interfacial properties, etc.) of materials are optimally combined, long lifespan and high efficiency may be achieved at the same time.

Meanwhile, it is necessary to develop a hole injection layer material having stable characteristics against Joule heating generated during device driving, that is, a high glass transition temperature while delaying the penetration and diffusion of metal oxide from the anode electrode (ITO) into the organic layer, which is one of the causes of shortening the lifespan of the organic electric device. Further, the low glass transition temperature of the hole transport layer material has a characteristic of lowering the uniformity of the thin film surface when driving the device, which is reported to have a significant effect on the device lifespan.

However, this cannot be achieved simply with the structural characteristics of the materials present in the organic material layer, and when the core and sub-substituent properties of each material and the appropriate combination between the respective organic material layers are achieved, a device with high efficiency and long lifespan may be realized.

Therefore, in order to sufficiently exhibit the excellent characteristics of the organic electric device, materials constituting the organic material layer in the device, for example, a hole injection material, a hole transport material, a light emitting material, an electron transport material, an electron injection material, a light emitting auxiliary layer material, etc. should be supported by stable and efficient materials in advance. In particular, there is an urgent need for the development of materials used for the hole transport layer, the light emitting auxiliary layer, etc.

### SUMMARY

An object of the present disclosure is to provide a compound capable of lowering the driving voltage of a device and improving the luminous efficiency, color purity and lifespan of the device, an organic electric device using the same, and an electronic device including the organic electric device.

In an aspect, the present disclosure provides a compound represented by Chemical Formula below.

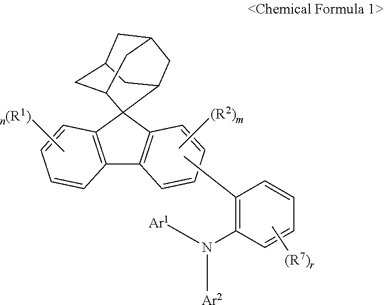

In another aspect, the present disclosure provides an organic electric device using the compound represented by Chemical Formula above and an electronic device thereof.

### Advantageous Effects

There are effects that high luminous efficiency and low driving voltage of the device can be achieved, and the color purity and lifespan of the device can be improved by using the compound according to the present disclosure.

### BRIEF DESCRIPTION OF THE DRAWINGS

FIGS. 1 to 3 schematically show organic electric devices according to embodiments of the present disclosure.

FIG. 4 shows a chemical formula according to an aspect of the present disclosure.

### DESCRIPTION OF EXEMPLARY EMBODIMENTS

The present disclosure provides a compound represented by Chemical Formula below.

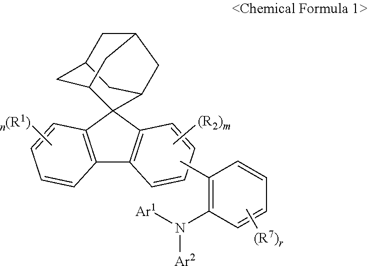

In another aspect, the present disclosure provides an organic electric device using the compound represented by Chemical Formula above and an electronic device thereof.

Hereinafter, preferred embodiments of the present disclosure will be described with reference to the accompanying drawings.

In order to describe the present embodiments, it should be noted that, in adding reference numerals to components of each drawing, the same components are given the same reference numerals as much as possible even though they are indicated in different drawings. Further, in describing the present disclosure, if it is determined that a detailed description of a related known configuration or function may obscure the gist of the present disclosure, the detailed description thereof will be omitted. In the drawings referenced below, no scale ratio applies.

In describing the components of the present disclosure, a term such as first, second, A, B, (a), (b), or the like may be used. Such a term is only for distinguishing the component from other components, and the essence, order, or sequence of the corresponding component is not limited by the term.

When it is described that a component is “linked”, “coupled” or “connected” to other component, it should be understood that the component may be directly linked or connected to the other component, but another component may also be “linked,” “coupled,” or “connected between the respective components.

Further, when an element such as a layer, a film, a region, a plate, or the like is said to be “above” or “on” other element, it should be understood that this includes not only the case where it is “directly above” the other element, but also the case where another element is in the middle therebetween. Conversely, it should be understood that when an element is said to be “directly on” other part, it means that there is no another part in the middle therebetween.

The terms used in the present specification and the appended claims are as follows, unless otherwise stated, within a range in which they are not departed from the spirit of the present disclosure.

The term “halo” or “halogen” used in the present application includes fluorine (F), chlorine (Cl), bromine (Br), and iodine (I) unless otherwise specified.

The term “alkyl” or “alkyl group” used in the present application, unless otherwise specified, has 1 to 60 carbons linked by a single bond, and means a radical of saturated aliphatic functional groups including a straight chain alkyl group, a branched chain alkyl group, a cycloalkyl (alicyclic) group, an alkyl-substituted cycloalkyl group, and a cycloalkyl-substituted alkyl group.

The term “haloalkyl group” or “halogen alkyl group” used in the present application refers to an alkyl group substituted with halogen unless otherwise specified.

The term “alkenyl” or “alkynyl” used in the present application has a double bond or a triple bond respectively, unless otherwise specified, contains a straight or branched chain group, and has 2 to 60 carbon atoms, but is not limited thereto.

The term “cycloalkyl” used in the present application refers to an alkyl forming a ring having 3 to 60 carbon atoms, unless otherwise specified, and is not limited thereto.

The term “alkoxy group” or “alkyloxy group” used in the present application refers to an alkyl group to which an oxygen radical is bonded, and has 1 to 60 carbon atoms unless otherwise specified, but is not limited thereto.

The terms “alkenoxyl group”, “alkenoxy group”, “alkenyloxyl group”, or “alkenyloxy group” used in the present application means an alkenyl group to which an oxygen radical is attached and, unless otherwise specified, has 2 to 60 carbon atoms, but is not limited thereto.

The terms “aryl group” and “arylene group” used in the present application have 6 to 60 carbon atoms respectively, unless otherwise specified, but are not limited thereto. In the present application, the aryl group or the arylene group includes a monocyclic type, ring assemblies, fused multiple cyclic compounds, and the like. For example, the aryl group may include a phenyl group, a monovalent functional group of biphenyl, a monovalent functional group of naphthalene, a fluorenyl group, and a substituted fluorenyl group, and the arylene group may include a fluorenylene group and a substituted fluorenylene group.

The term “ring assemblies” used in the present application means that two or more ring systems (monocyclic or bonded ring systems) are directly linked to each other through a single bond or a double bond, and the number of direct links between such rings is one less than the total number of ring systems contained in the compound. In the ring assemblies, the same or different ring systems may be directly linked to each other through a single bond or a double bond.

Since the aryl group in the present application includes a ring assembly, the aryl group includes biphenyl and terphenyl in which a benzene ring that is an aromatic single ring is linked by a single bond. Further, since the aryl group also includes a compound in which an aromatic ring system fused to an aromatic single ring is linked by a single bond, it also includes, for example, a compound in which a fluorene, an aromatic ring system fused to a benzene ring that is an aromatic single ring, is linked by a single bond.

The term “fused multiple ring systems” used in the present application refers to a fused ring form that shares at least two atoms, and includes a form in which a ring system of two or more hydrocarbons is fused and a form in which at least one heterocyclic system containing at least one heteroatom is fused. Such fused multiple ring systems may be aromatic rings, heteroaromatic rings, aliphatic rings, or combinations of these rings. For example, in the case of an aryl group, it may be a naphthalenyl group, a phenanthrenyl group, a fluorenyl group, etc., but is not limited thereto.

The term “spiro compound” used in the present application has a ‘spiro union’, and the spiro union refers to a connection formed by two rings sharing only one atom. At this time, the atoms shared by the two rings are called ‘spiro atoms’, and these are respectively called ‘a monospiro-compound’, ‘a dispiro-compound’, and ‘a trispiro-compound’ depending on the number of spiro atoms contained in a compound.

The term “fluorenyl group”, “fluorenylene group”, or “fluorentriyl group” used in the present application mean that it is a monovalent, divalent or trivalent functional group in which R, R′, R″ and R′″ are all hydrogen in the structure below respectively, unless otherwise specified, and “a substituted fluorenyl group”, “a substituted fluorenylene group” or “a substituted fluorentriyl group” refers to one in which at least one of substituents R, R′, R″, and R′″ is a substituent other than hydrogen, and includes cases in which R and R′ are bonded to each other to form a spiro compound together with carbon to which they are bonded. In the present specification, the fluorenyl group, the fluorenylene group, and the fluorentriyl group may all be referred to as fluorene groups regardless of valences such as monovalent, divalent, trivalent, etc.

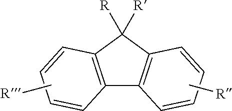

Further, R, R′, R″ and R′″ may be each independently an alkyl group having 1 to 20 carbon atoms, an alkenyl group having 1 to 20 carbon atoms, an aryl group having 6 to 30 carbon atoms, or a heterocyclic group having 3 to 30 carbon atoms, and for example, the aryl group may be phenyl, biphenyl, naphthalene, anthracene, or phenanthrene, and the heterocyclic group may be pyrrole, furan, thiophene, pyrazole, imidazole, triazole, pyridine, pyrimidine, pyridazine, pyrazine, triazine, indole, benzofuran, quinazoline, or quinoxaline. For example, the substituted fluorenyl group and the fluorenylene group may be a monovalent functional group or a divalent functional group of 9,9-dimethylfluorene, 9,9-diphenylfluorene, and 9,9′-spirobi[9H-fluorene] respectively.

The term “heterocyclic group” used in the present application includes not only aromatic rings such as “heteroaryl group” or “heteroarylene group” but also non-aromatic rings, and unless otherwise specified, it means a ring of 2 to 60 carbon atoms which each include one or more heteroatoms, but is not limited thereto. The term “heteroatom” used in the present application refers to N, O, S, P, or Si, unless otherwise specified, and the heterocyclic group means a monocyclic type, ring assemblies, fused multiple ring systems, spiro compounds, or the like containing heteroatoms.

For example, the “heterocyclic group” may also include compounds containing a heteroatom group such as SO₂, P═O, etc., such as the compound below instead of carbon forming a ring.

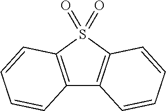 [SMILES: O=S1(=O)c2ccccc2-c2ccccc21]

The term “ring” used in the present application includes monocyclic and polycyclic rings, includes heterocycles containing at least one heteroatom as well as hydrocarbon rings, and includes aromatic and non-aromatic rings.

The term “polycyclic” used in the present application includes ring assemblies such as biphenyl, terphenyl, etc., fused multiple ring systems, and spiro compounds, includes non-aromatic groups as well as aromatic groups, and includes at least one heteroatom-containing heterocycles as well as hydrocarbon rings.

The term “aliphatic ring group” used in the present application means a cyclic hydrocarbon other than an aromatic hydrocarbon, and includes a monocyclic type, ring assemblies, fused multiple ring systems, spiro compounds, etc., and unless otherwise specified, it means a ring of 3 to 60 carbon atoms, but is not limited thereto. For example, even when benzene, which is an aromatic ring, and cyclohexane, which is a non-aromatic ring, are fused, it corresponds to an aliphatic ring.

Further, when prefixes are named consecutively, it is meant that the substituents are listed in the order listed first. For example, an arylalkoxy group means an alkoxy group substituted with an aryl group, an alkoxycarbonyl group means a carbonyl group substituted with an alkoxy group, and an arylcarbonylalkenyl group means an alkenyl group substituted with an arylcarbonyl group, where the arylcarbonyl group is a carbonyl group substituted with an aryl group.

Further, unless otherwise explicitly stated, the “substitution” in the term “substituted or unsubstituted” used in the present application means being substituted with one or more substituents selected from the group consisting of deuterium, halogen, an amino group, a nitrile group, a nitro group, a C₁-C₂₀ alkyl group, a C₁-C₂₀ alkoxy group, a C₁-C₂₀ alkylamine group, a C₁-C₂₀ alkylthiophene group, a C₆-C₂₀ arylthiophene group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted with deuterium, a C₈-C₂₀ arylalkenyl group, a silane group, a boron group, a germanium group, and a C₂-C₂₀ heterocyclic group containing at least one heteroatom selected from the group consisting of O, N, S, Si, and P, but the substituents are not limited to these substituents.

In the present application, the ‘functional group name’ corresponding to the aryl group, arylene group, heterocyclic group, etc. exemplified as examples of each symbol and its substituents may be described as ‘the name of the functional group reflecting the valence’, but it may also be described as ‘the name of the parent compound’. For example, in the case of ‘phenanthrene’ that is a type of aryl group, the name of the group may be described by dividing the valency, such as dividing a monovalent ‘group’ into ‘phenanthryl (group)’ and dividing a divalent group into ‘phenanthrylene (group)’, but it may also be described as ‘phenanthrene’, which is the name of the parent compound, regardless of the valence.

Similarly, in the case of pyrimidine, it may be described as ‘pyrimidine’ regardless of the valence, or it may be described as the ‘name of the group’ of the corresponding valence, such as describing it as a pyrimidinyl (group) if it is a monovalent and describing it as a pyrimidinylene (group) if it is a divalent. Accordingly, when the type of the substituent is described as the name of the parent compound in the present application, it may mean an n-valent ‘group’ formed by the detachment of a hydrogen atom bonding to a carbon atom and/or a heteroatom of the parent compound.

Further, numbers or alphabets indicating positions in describing the compound name or the substituent name may be omitted in the present specification. For example, pyrido[4,3-d]pyrimidine may be described as pyridopyrimidine, benzofuro[2,3-d]pyrimidine may be described as benzofuropyrimidine, and 9,9-dimethyl-9H-fluorene may be described as dimethylfluorene. Therefore, both benzo[g]quinoxaline and benzo[f]quinoxaline may be described as benzoquinoxaline.

Further, unless there is an explicit explanation, Chemical Formulas used in the present application are applied in the same way as the definition of the substituent by the exponent definition of Chemical Formula below.

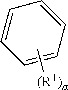

Here, when a is an integer of 0, it means that the substituent R¹ does not exist, that is, when a is 0, it means that hydrogen is bonded to all of carbons forming the benzene ring, and in this case, the indication of hydrogen bonded to carbons may be omitted, and the chemical formula or compound may be described. Further, one substituent R¹ may be bonded to any one of carbons forming the benzene ring when a is an integer of 1, it may be bonded, for example, as follows when a is an integer of 2 or 3, it may be bonded to carbons of the benzene ring in a similar manner even when a is an integer of 4 to 6, and R¹ may be the same as or different from each other when a is an integer of 2 or more.

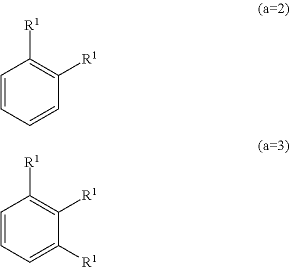

Unless otherwise specified in the present application, forming a ring means that adjacent groups are bonded to each other to form a single ring or fused multiple rings, and the single ring and the formed fused multiple rings may include heterocycles containing at least one heteroatom as well as hydrocarbon rings, and may include aromatic and non-aromatic rings.

Further, unless otherwise specified in the present specification, when representing a condensed ring, the number in ‘number-condensed ring’ indicates the number of rings to be condensed. For example, a form in which three rings such as anthracene, phenanthrene, benzoquinazoline, etc. are condensed with each other may be expressed as a 3-condensed ring.

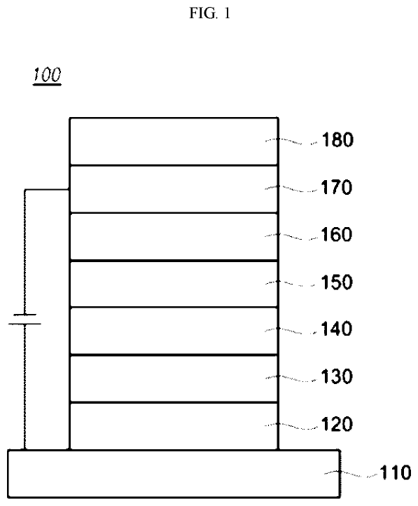

Hereinafter, the stacked structure of the organic electric device comprising the compound of the present disclosure will be described with reference to FIGS. 1 to 3.

Referring to FIG. 1, the organic electric device 100 according to an embodiment of the present disclosure comprises a first electrode 110 formed on a substrate (not shown), a second electrode 170, and an organic material layer containing the compound according to the present disclosure between the first electrode 110 and the second electrodes 170.

The first electrode 110 may be an anode, the second electrode 170 may be a cathode, and in the case of an inverted type, the first electrode may be a cathode, and the second electrode may be an anode.

The organic material layer may include a hole injection layer 120, a hole transport layer 130, a light emitting layer 140, an electron transport layer 150, and an electron injection layer 160. Specifically, the hole injection layer 120, the hole transport layer 130, the light emitting layer 140, the electron transport layer 150, and the electron injection layer 160 may be sequentially formed on the first electrode 110.

Preferably, a capping layer 180 may be formed on one surface of both surfaces of the first electrode 110 or the second electrode 170 that is not in contact with the organic material layer, and the light efficiency of the organic electric device may be improved when the capping layer 180 is formed.

For example, the capping layer 180 may be formed on the second electrode 170. In the case of a top emission organic light emitting device, the capping layer 180 is formed so that optical energy loss due to surface plasmon polaritons (SPPs) in the second electrode 170 may be reduced, and in the case of a bottom emission organic light emitting device, the capping layer 180 may serve to buffer the second electrode 170.

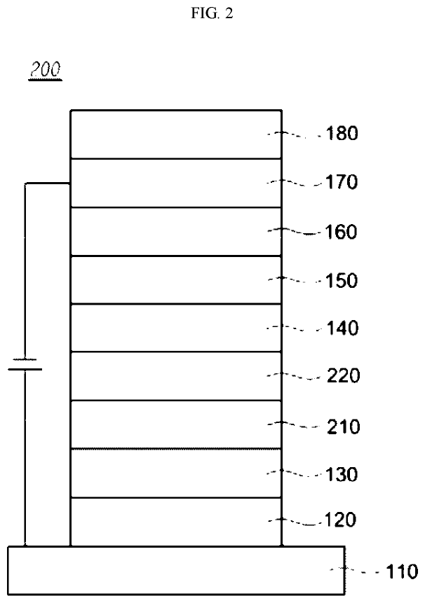

Meanwhile, a buffer layer 210 or a light emitting auxiliary layer 220 may be further formed between the hole transport layer 130 and the light emitting layer 140, which will be described with reference to FIG. 2.

Referring to FIG. 2, the organic electric device 200 according to another embodiment of the present disclosure may comprise a hole injection layer 120, a hole transport layer 130, a buffer layer 210, a light emitting auxiliary layer 220, a light emitting layer 140, an electron transport layer 150, an electron injection layer 160, and a second electrode 170 which are sequentially formed on a first electrode 110, and a capping layer 180 may be formed on the second electrode.

Although it is not shown in FIG. 2, an electron transport auxiliary layer may be further formed between the light emitting layer 140 and the electron transport layer 150.

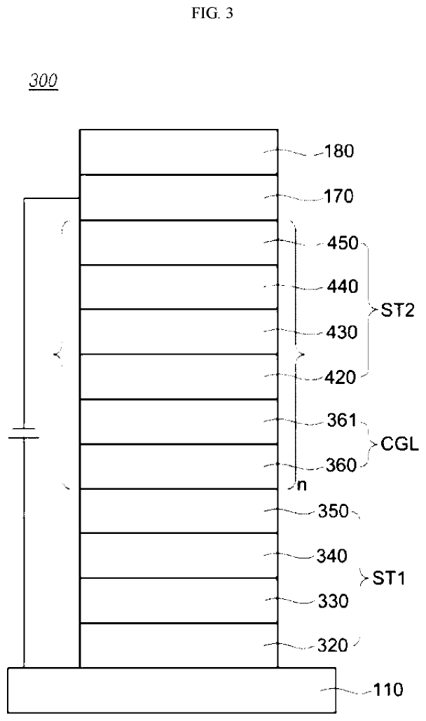

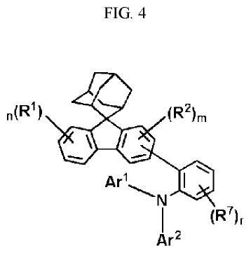

Further, according to another embodiment of the present disclosure, the organic material layer may be a form in which a plurality of stacks including a hole transport layer, a light emitting layer, and an electron transport layer are formed. This will be described with reference to FIG. 3.

Referring to FIG. 3, the organic electric device 300 according to another embodiment of the present disclosure may comprise two sets or more stacks ST1 and ST2 of an organic material layer formed as a multilayer between the first electrode 110 and the second electrode 170, and a charge generation layer CGL may be formed between the stacks of the organic material layer.

Specifically, the organic electric device according to an embodiment of the present disclosure may comprise a first electrode 110, a first stack ST1, a charge generation layer CGL, a second stack ST2, and a second electrode 170, and a capping layer 180.

The first stack ST1 is an organic material layer formed on the first electrode 110, which may contain a first hole injection layer 320, a first hole transport layer 330, a first light emitting layer 340, and a first electron transport layer 350.

The second stack ST2 may contain a second hole injection layer 420, a second hole transport layer 430, a second light emitting layer 440, and a second electron transport layer 450.

As described above, the first stack and the second stack may be organic material layers having the same stacked structure, but may be organic material layers having different stacked structures.

A charge generation layer CGL may be formed between the first stack ST1 and the second stack ST2. The charge generation layer CGL may include a first charge generation layer 360 and a second charge generation layer 361. Such a charge generation layer CGL is formed between the first light emitting layer 340 and the second light emitting layer 440 to play a role of increasing the efficiency of current generated in each light emitting layer and smoothly distributing electric charges.

The first light emitting layer 340 may contain a light emitting material including a blue fluorescent dopant in a blue host, and the second light emitting layer 440 may contain a material doped together with a greenish yellow dopant and a red dopant in a green host, but the materials of the first light emitting layer 340 and the second light emitting layer 440 according to the embodiment of the present disclosure are not limited thereto.

In this case, the second hole transport layer 430 is formed by containing the second stack ST2 in which the energy level is set to be higher than the triplet excited state energy level of the second light emitting layer 440.

Since the energy level of the second hole transport layer 430 is higher than that of the second light emitting layer 440, it can be prevented that triplet excitons of the second light emitting layer 440 come over to the second hole transport layer 430 to lower the luminous efficiency. That is, the second hole transport layer 430 may function as an exciton blocking layer that simultaneously functions to transport holes from the inherent second light emitting layer 440 and prevents triplet excitons from coming over.

Further, for the function of the exciton blocking layer, the first hole transport layer 330 may also be set to an energy level higher than the triplet excitation energy level of the first light emitting layer 340. In addition, it is preferable that the first electron transport layer 350 is also set to an energy level higher than the energy level of the triplet excited state of the first light emitting layer 340, and the second electron transport layer 450 is also set to an energy level higher than the energy level of the triplet excited state of the second light emitting layer 440.

In FIG. 3, n may be an integer of 1 to 5. When n is 2, the charge generation layer CGL and the third stack may be additionally stacked on the second stack ST2.

When a plurality of light emitting layers are formed by the multilayer stack structure method as shown in FIG. 3, not only an organic electroluminescent device that emits white light by the mixing effect of light emitted from each light emitting layer can be manufactured, but also an organic electroluminescent device that emits light of various colors can be manufactured.

The compound represented by Chemical Formula 1 of the present disclosure may be used as a material of the hole injection layers 120, 320, and 420, the hole transport layers 130, 330, and 430, the buffer layer 210, the light emitting auxiliary layer 220, the electron transport layers 150, 350, and 450, the electron injection layer 160, the light emitting layers 140, 340, and 440, or the capping layer 180, but may be preferably used as a material of the hole transport layers 130, 330, and 430, the light emitting auxiliary layer 220, the light emitting layers 140, 340, and 440, and/or the capping layer 180.

The organic electric devices according to FIGS. 1 to 3 may further comprise a protective layer (not shown) and an encapsulation layer (not shown). The protective layer may be positioned on the capping layer, and the encapsulation layer may be positioned on the capping layer, and may be formed to cover a side portion of one or more of the first electrode, the second electrode, and the organic material layer in order to protect the first electrode, the second electrode, and the organic material layer.

The protective layer may provide a planarized surface so that the encapsulation layer is uniformly formed, and may serve to protect the first electrode, the second electrode, and the organic material layer during the manufacturing process of the encapsulation layer.

The encapsulation layer may serve to prevent the penetration of external oxygen and moisture into the organic electric device.

Meanwhile, even when it is the same or similar core, the band gap, electrical properties, interfacial properties, etc. may vary depending on which substituent is bonded at which position. Therefore, it is necessary to study the selection of the core and the combination of sub-substituents bound thereto. In particular, when the energy level and T1 value between the respective organic material layers, the intrinsic properties (mobility, interfacial properties, etc.) of a material, and the like are optimally combined, long lifespan and high efficiency may be achieved at the same time.

Therefore, in the present disclosure, the lifespan and efficiency of the organic electric device could be improved at the same time by using the compound represented by Chemical Formula 1 as a material for the light emitting auxiliary layer 220, the light emitting layers 140, 340, and 440, and/or the capping layer 180, thereby optimizing the energy level and T1 value between the respective organic material layers, the intrinsic properties (mobility, interfacial properties, etc.) of the material, and the like.

The organic electroluminescent device according to an embodiment of the present disclosure may be manufactured using various deposition methods. It may be manufactured using a deposition method such as PVD or CVD. For example, it may be manufactured by depositing a metal or a metal oxide having conductivity or an alloy thereof on a substrate to form the anode 110, forming the organic material layer including the hole injection layers 120, 320 and 420, the hole transport layers 130, 330, and 430, the light emitting layers 140, 340, and 440, the electron transport layers 150, 350, and 450, and the electron injection layer 160 thereon, and then depositing a material that can be used as the cathode 170 thereon. Further, the light emitting auxiliary layer 220 may be further formed between the hole transport layers 130, 330, and 430 and the light emitting layers 140, 340, and 440, and an electron transport auxiliary layer (not shown) may be further formed between the light emitting layer 140 and the electron transport layer 150, or they may be formed in a stack structure as described above.

Further, the organic material layer may be manufactured into a smaller number of layers by a solution process or a solvent process rather than a deposition method using various polymer materials, such as a spin coating process, a nozzle printing process, an inkjet printing process, a slot coating process, a dip coating process, a roll-to-roll process, a doctor blading process, a screen printing process, or a method such as a thermal transfer method or the like. Since the organic material layer according to the present disclosure may be formed by various methods, the scope of rights of the present disclosure is not limited by the formation methods.

The organic electric device according to an embodiment of the present disclosure may be a top emission type, a back emission type, or a double-sided emission type depending on the material used.

The organic electric device according to an embodiment of the present disclosure may include an organic electroluminescent device, an organic solar cell, an organic photoreceptor, an organic transistor, a device for monochromatic lighting, a device for a quantum dot display, and the like.

Another embodiment of the present disclosure may include a display device including the organic electric device of the present disclosure described above, and an electronic device comprising a control unit for controlling the display device. In this case, the electronic device may be a current or future wired/wireless communication terminal, and includes all electronic devices such as a mobile communication terminal such as a mobile phone, a PDA, an electronic dictionary, a PMP, a remote controller, a navigation system, a game machine, various TVs, and various computers.

Hereinafter, the compound according to one aspect of the present disclosure will be described.

The compound according to one aspect of the present disclosure is represented by Chemical Formula 1 below.

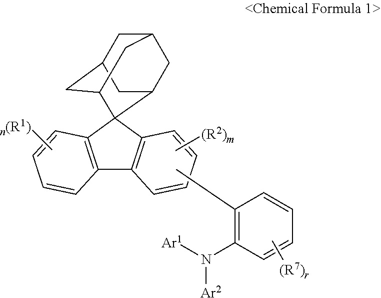

In Chemical Formula 1,

1) Ar¹ to Ar² are each independently a C₆-C₆₀ aryl group; a fluorenyl group; a C₂-C₆₀ heterocyclic group containing at least one heteroatom of O, N, S, Si, and P; or a fused ring group of a C₃-C₆₀ aliphatic ring and a C₆-C₆₀ aromatic ring; a C₁-C₃₀ alkyl group; a C₂-C₃₀ alkenyl group; a C₂-C₃₀ alkynyl group; a C₁-C₃₀ alkoxyl group; a C₆-C₃₀ aryloxy group; -Lₐ-N(Rₐ)(R_{b}); or combinations thereof,

2) at least one of Ar¹ and Ar² may be represented by Chemical Formula 1-a or Chemical Formula 1-b below,

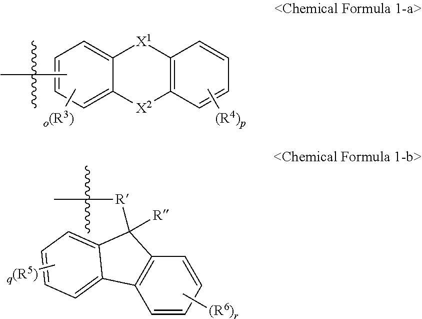

3) R¹ to R⁶, R′, and R″ are each independently hydrogen; deuterium; halogen; a cyano group; a nitro group; a C₆-C₆₀ aryl group; a fluorenyl group; a C₂-C₆₀ heterocyclic group containing at least one heteroatom of O, N, S, Si, and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; or -Lₐ-N(Rₐ)(R_{b}); or adjacent groups may be bonded to each other to form a ring,

4) Rₐ to R_{b} are each independently a C₆-C₆₀ aryl group; a fluorenyl group; a C₂-C₆₀ heterocyclic group containing at least one heteroatom of O, N, S, Si, and P; or a fused ring group of a C₃-C₆₀ aliphatic ring and a C₆-C₆₀ aromatic ring; or combinations thereof,

5) Lₐ is a single bond; a C₆-C₆₀ arylene group; a fluorenylene group; a C₂-C₆₀ heterocyclic group containing at least one heteroatom of O, N, S, Si, and P; a fused ring group of a C₃-C₆₀ aliphatic ring and a C₆-C₆₀ aromatic ring; or combinations thereof,

6) R⁷ is hydrogen; deuterium; halogen; a cyano group; a nitro group; a C₆-C₆₀ aryl group; a fluorenyl group; a C₂-C₆₀ heterocyclic group containing at least one heteroatom of O, N, S, Si, and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; or a C₆-C₃₀ aryloxy group; or adjacent groups may be bonded to each other to form a ring,

7) X¹ is CR′R″, NR′, O, S, Se, or SiR′R″,

8) X² is CR′R″, NR′, O, S, Se, SiR′R″, or a single bond,

9) n, p, q, and r are each independently an integer of 0 to 4,

10) m and o are each independently an integer of 0 to 3,

11) Ar¹ to Ar², R¹ to R⁷, R′, R″, Rₐ to R_{b}, Lₐ, and the rings formed by bonding neighboring groups to each other may each be further substituted with one or more substituents selected from the group consisting of deuterium; halogen; a silane group substituted or unsubstituted with a C₁-C₂₀ alkyl group or a C₆-C₂₀ aryl group; a siloxane group; a boron group; a germanium group; a cyano group; an amino group; a nitro group; a C₁-C₂₀ alkylthio group; a C₁-C₂₀ alkoxy group; a C₆-C₂₀ arylalkoxy group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group containing at least one heteroatom selected from the group consisting of O, N, S, Si, and P; a C₃-C₂₀ aliphatic ring group; a C₇-C₂₀ arylalkyl group; a C₈-C₂₀ arylalkenyl group; and combinations thereof, and adjacent substituents may form a ring with each other.

When Ar¹ to Ar², R¹ to R⁷, R′, R″, and Rₐ to R_{b} are an aryl group, they may be preferably a C₆-C₃₀ aryl group, more preferably a C₆-C₁₈ aryl group, such as phenyl, biphenyl, naphthyl, terphenyl, or the like.

When Ar¹ to Ar², R¹ to R⁷, R′, R″, and Rₐ to R_{b} are a heterocyclic group, they may be preferably a C₂-C₃₀ heterocyclic group, more preferably a C₂-C₁₈ heterocyclic group, such as dibenzofuran, dibenzothiophene, naphthobenzothiophene, naphthobenzofuran, or the like.

When Ar¹ to Ar², R¹ to R⁷, R′, R″, and Rₐ to R_{b} are a fluorenyl group, they may be preferably 9,9-dimethyl-9H-fluorene, 9,9-diphenyl-9H-fluorenyl group, 9,9′-spirobifluorene, or the like.

When Lₐ is an arylene group, it may be preferably a C₆-C₃₀ arylene group, more preferably a C₆-C₁₈ arylene group, such as phenyl, biphenyl, naphthyl, terphenyl, or the like.

When R¹ to R⁷, R′, and R″ are an alkyl group, they may be preferably a C₁-C₁₀ alkyl group, such as methyl, t-butyl, or the like.

When R¹ to R⁷, R′, and R″ are an alkoxyl group, they may be preferably a C₁-C₂₀ alkoxyl group, more preferably a C₁-C₁₀ alkoxyl group, such as methoxy, t-butoxy, or the like.

The rings formed by bonding to each other adjacent groups of Ar¹ to Ar², R¹ to R⁷, R′, R″, Rₐ to R_{b}, and Lₐ may be a C₆-C₆₀ aromatic ring group; a fluorenyl group; a C₂-C₆₀ heterocyclic group containing at least one heteroatom of O, N, S, Si, and P; or a C₃-C₆₀ aliphatic ring group, and for example, when adjacent groups are bonded to each other to form an aromatic ring, preferably a C₆-C₂₀ aromatic ring, more preferably a C₆-C₁₄ aromatic ring, such as benzene, naphthalene, phenanthrene, or the like may be formed.

Chemical Formula 1 above may be represented by any one of Chemical Formulas 2 to 4 below, but is not limited thereto.

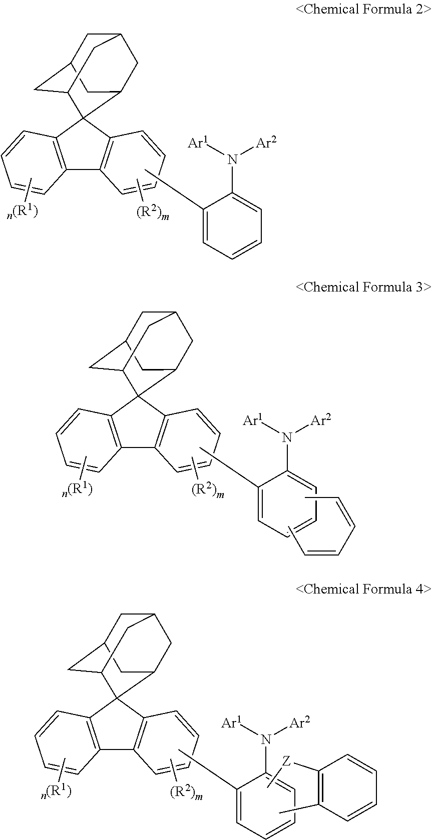

In Chemical Formulas 2 to 4,

1) Z is CR′R″, NR′, O, S, Se, SiR′R″, or a single bond, and

2) Ar¹ to Ar², R′, R″, and R¹ to R² are the same as defined in Chemical Formula 1.

Chemical Formula 1 above may be represented by Chemical Formula 5 or Chemical Formula 6 below, but is not limited thereto.

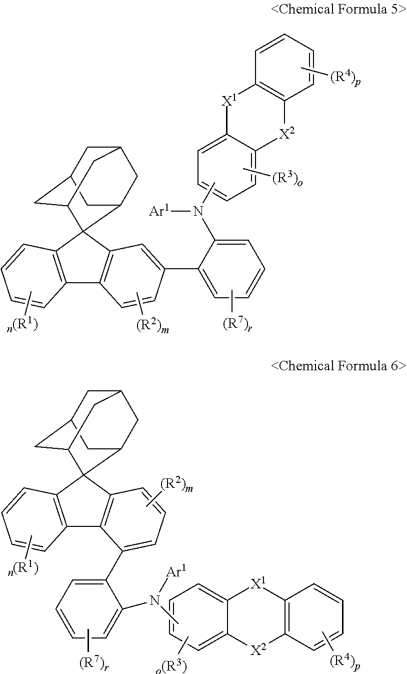

In Chemical Formula 5 and Chemical Formula 6,

X¹, X², Ar¹, R¹ to R⁴, R⁷, m to p, and r are the same as defined in Chemical Formula 1.

Chemical Formula 1 above may be represented by Chemical Formula 7 or Chemical Formula 8 below, but is not limited thereto.

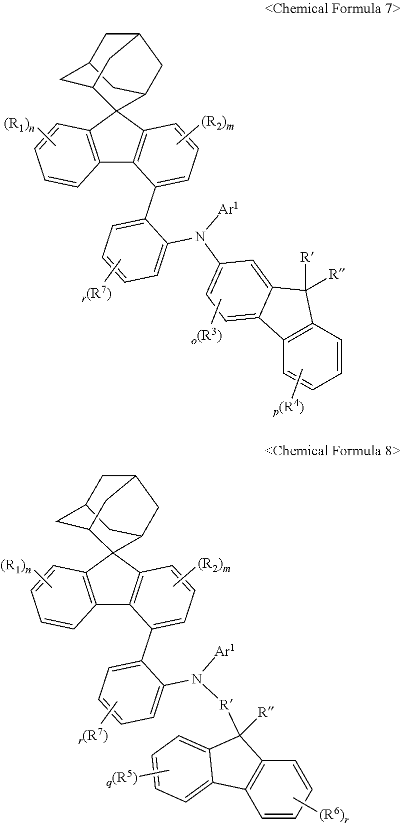

In Chemical Formulas 7 and 8,

Ar¹, R¹ to R⁷, R′, R″, and m to r are the same as defined in Chemical Formula 1.

Chemical Formula 1-a may be represented by any one of Chemical Formulas 1-a-1 to 1-a-5 below, but is not limited thereto.

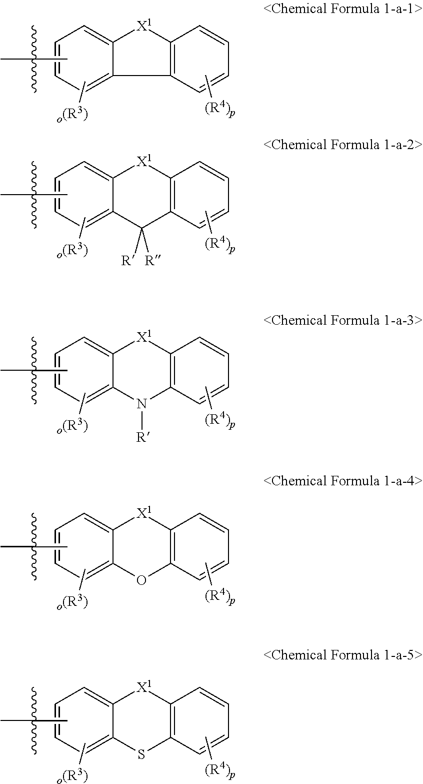

In Chemical Formula 1-a-1 to Chemical Formula 1-a-5,

X¹, R³ to R⁴, R′, R″, o, and p are the same as defined in Chemical Formula 1.

Meanwhile, the compound represented by Chemical Formula 1 above may be one of P-1 to P-90 below, but is not limited thereto.

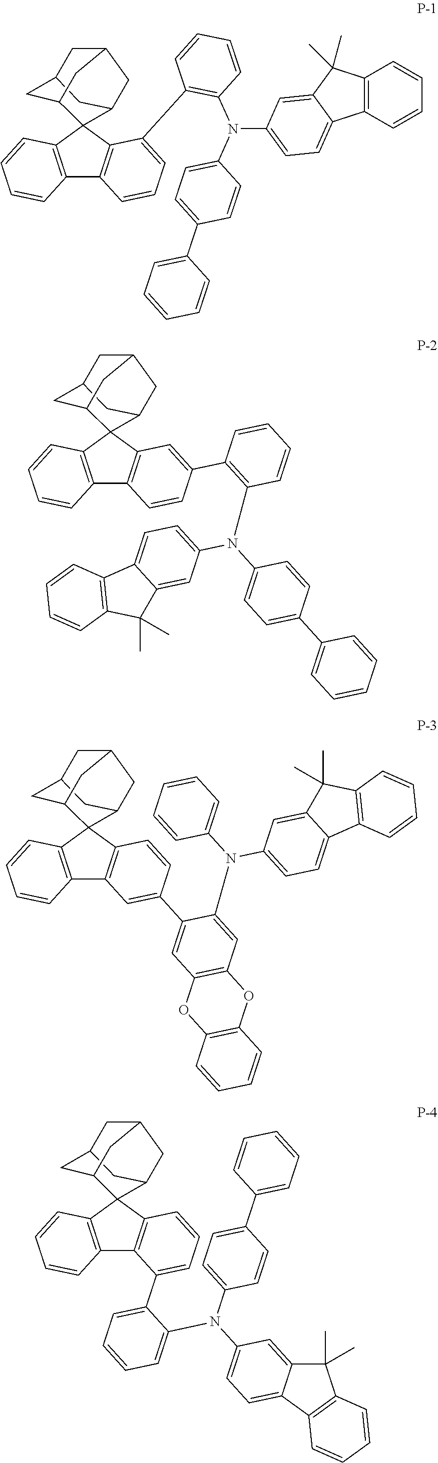 [SMILES: CC1(C)c2ccccc2-c2ccc(N(c3ccc(-c4ccccc4)cc3)c3ccccc3-c3cccc4c3C3(c5ccccc5-4)C4CC5CC(C4)CC3C5)cc21 ; CC1(C)c2ccccc2-c2ccc(N(c3ccc(-c4ccccc4)cc3)c3ccccc3-c3ccc4c(c3)C3(c5ccccc5-4)C4CC5CC(C4)CC3C5)cc21 ; CC1(C)c2ccccc2-c2ccc(N(c3ccccc3)c3cc4c(cc3-c3ccc5c(c3)-c3ccccc3C53C5CC6CC(C5)CC3C6)Oc3ccccc3O4)cc21 ; CC1(C)c2ccccc2-c2ccc(N(c3ccc(-c4ccccc4)cc3)c3ccccc3-c3cccc4c3-c3ccccc3C43C4CC5CC(C4)CC3C5)cc21]

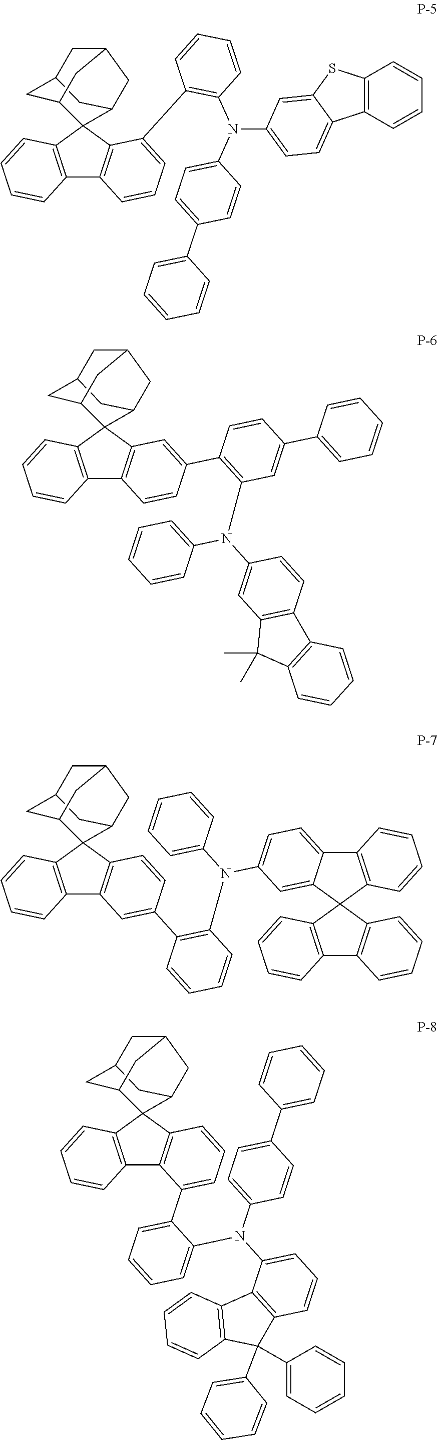 [SMILES: c1ccc(-c2ccc(N(c3ccc4c(c3)sc3ccccc34)c3ccccc3-c3cccc4c3C3(c5ccccc5-4)C4CC5CC(C4)CC3C5)cc2)cc1 ; CC1(C)c2ccccc2-c2ccc(N(c3ccccc3)c3cc(-c4ccccc4)ccc3-c3ccc4c(c3)C3(c5ccccc5-4)C4CC5CC(C4)CC3C5)cc21 ; c1ccc(N(c2ccc3c(c2)C2(c4ccccc4-c4ccccc42)c2ccccc2-3)c2ccccc2-c2ccc3c(c2)-c2ccccc2C32C3CC4CC(C3)CC2C4)cc1 ; c1ccc(-c2ccc(N(c3ccccc3-c3cccc4c3-c3ccccc3C43C4CC5CC(C4)CC3C5)c3cccc4c3-c3ccccc3C4(c3ccccc3)c3ccccc3)cc2)cc1]

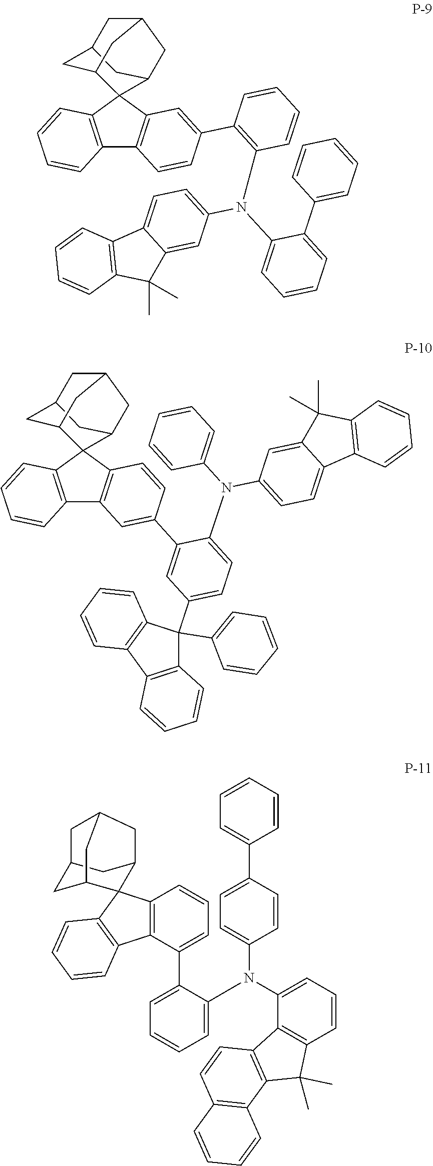 [SMILES: CC1(C)c2ccccc2-c2ccc(N(c3ccccc3-c3ccccc3)c3ccccc3-c3ccc4c(c3)C3(c5ccccc5-4)C4CC5CC(C4)CC3C5)cc21 ; CC1(C)c2ccccc2-c2ccc(N(c3ccccc3)c3ccc(C4(c5ccccc5)c5ccccc5-c5ccccc54)cc3-c3ccc4c(c3)-c3ccccc3C43C4CC5CC(C4)CC3C5)cc21 ; CC1(C)c2cccc(N(c3ccc(-c4ccccc4)cc3)c3ccccc3-c3cccc4c3-c3ccccc3C43C4CC5CC(C4)CC3C5)c2-c2ccc3ccccc3c21]

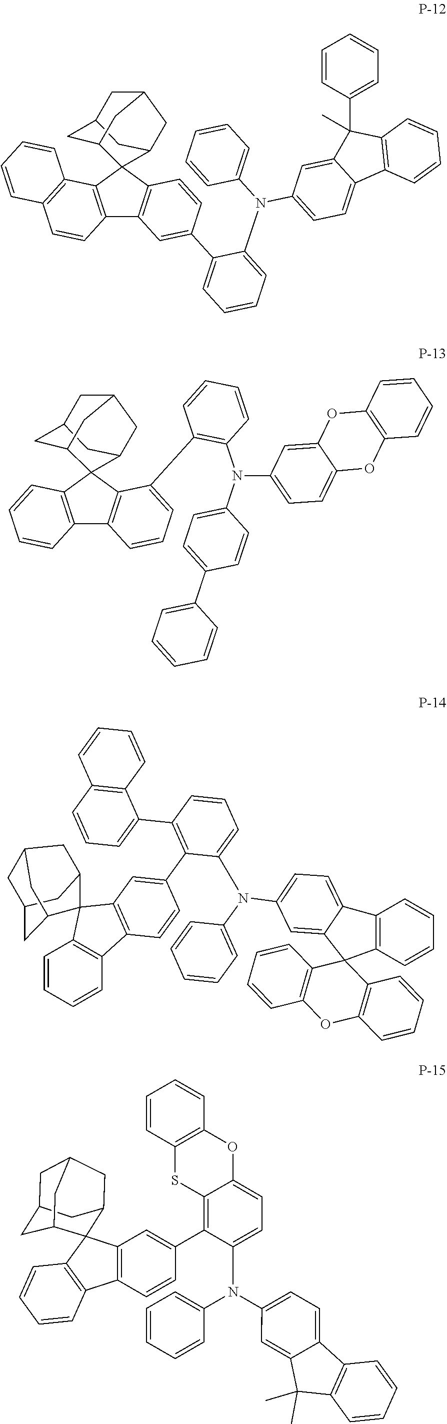 [SMILES: CC1(c2ccccc2)c2ccccc2-c2ccc(N(c3ccccc3)c3ccccc3-c3ccc4c(c3)-c3ccc5ccccc5c3C43C4CC5CC(C4)CC3C5)cc21 ; c1ccc(-c2ccc(N(c3ccc4c(c3)Oc3ccccc3O4)c3ccccc3-c3cccc4c3C3(c5ccccc5-4)C4CC5CC(C4)CC3C5)cc2)cc1 ; c1ccc(N(c2ccc3c(c2)C2(c4ccccc4Oc4ccccc42)c2ccccc2-3)c2cccc(-c3cccc4ccccc34)c2-c2ccc3c(c2)C2(c4ccccc4-3)C3CC4CC(C3)CC2C4)cc1 ; CC1(C)c2ccccc2-c2ccc(N(c3ccccc3)c3ccc4c(c3-c3ccc5c(c3)C3(c6ccccc6-5)C5CC6CC(C5)CC3C6)Sc3ccccc3O4)cc21]

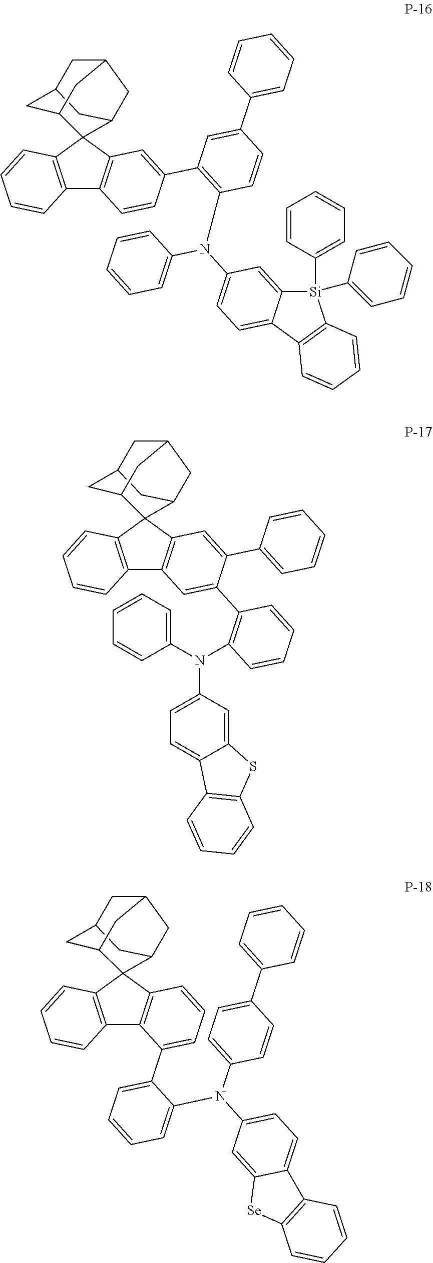 [SMILES: c1ccc(-c2ccc(N(c3ccccc3)c3ccc4c(c3)[Si](c3ccccc3)(c3ccccc3)c3ccccc3-4)c(-c3ccc4c(c3)C3(c5ccccc5-4)C4CC5CC(C4)CC3C5)c2)cc1 ; c1ccc(-c2cc3c(cc2-c2ccccc2N(c2ccccc2)c2ccc4c(c2)sc2ccccc24)-c2ccccc2C32C3CC4CC(C3)CC2C4)cc1 ; c1ccc(-c2ccc(N(c3ccc4c(c3)[se]c3ccccc34)c3ccccc3-c3cccc4c3-c3ccccc3C43C4CC5CC(C4)CC3C5)cc2)cc1]

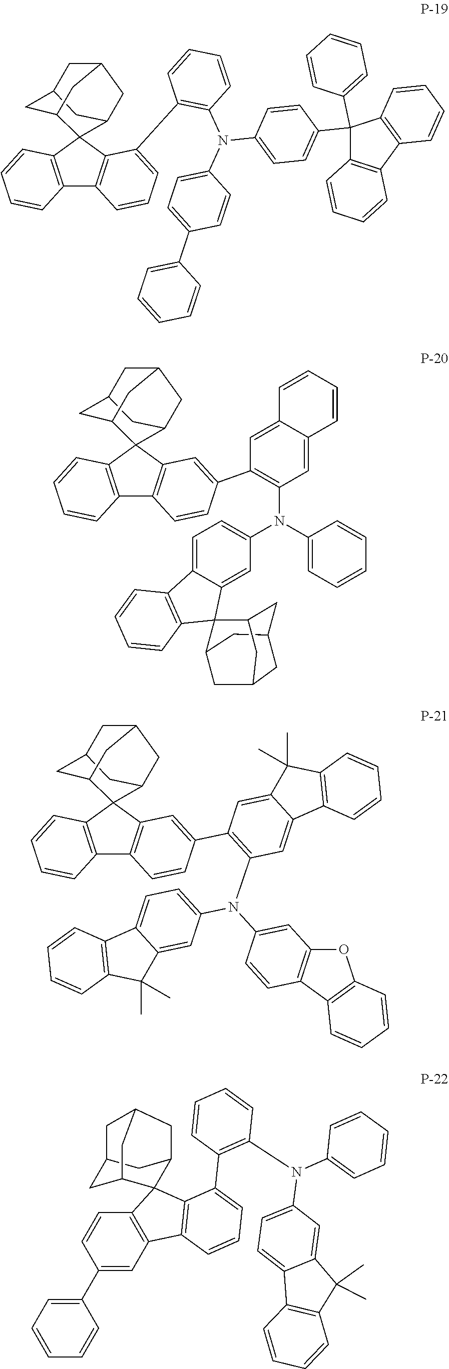

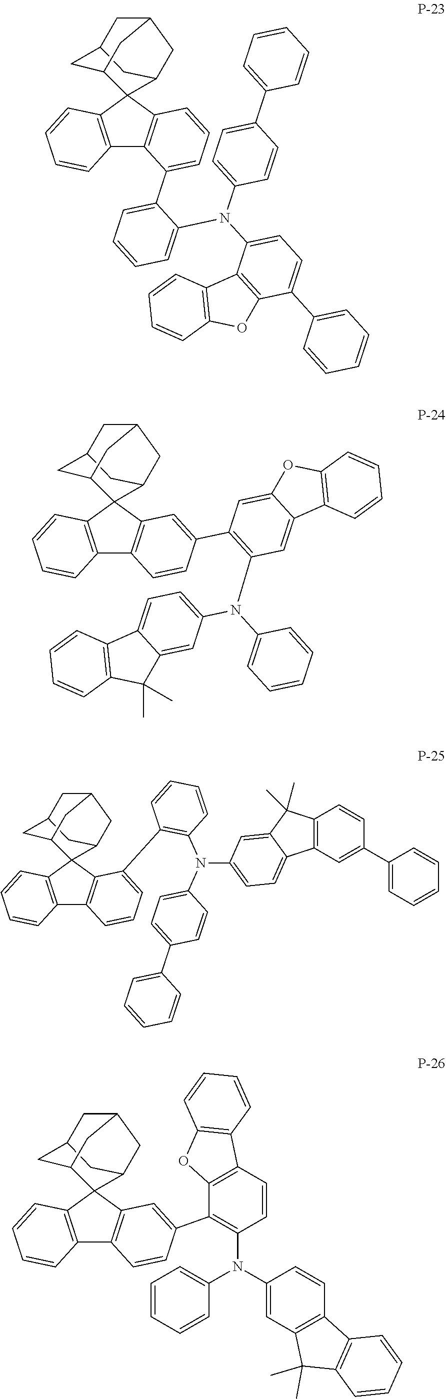 [SMILES: c1ccc(-c2ccc(N(c3ccccc3-c3cccc4c3-c3ccccc3C43C4CC5CC(C4)CC3C5)c3ccc(-c4ccccc4)c4oc5ccccc5c34)cc2)cc1 ; CC1(C)c2ccccc2-c2ccc(N(c3ccccc3)c3cc4c(cc3-c3ccc5c(c3)C3(c6ccccc6-5)C5CC6CC(C5)CC3C6)oc3ccccc34)cc21 ; CC1(C)c2ccc(-c3ccccc3)cc2-c2ccc(N(c3ccc(-c4ccccc4)cc3)c3ccccc3-c3cccc4c3C3(c5ccccc5-4)C4CC5CC(C4)CC3C5)cc21 ; CC1(C)c2ccccc2-c2ccc(N(c3ccccc3)c3ccc4c(oc5ccccc54)c3-c3ccc4c(c3)C3(c5ccccc5-4)C4CC5CC(C4)CC3C5)cc21]

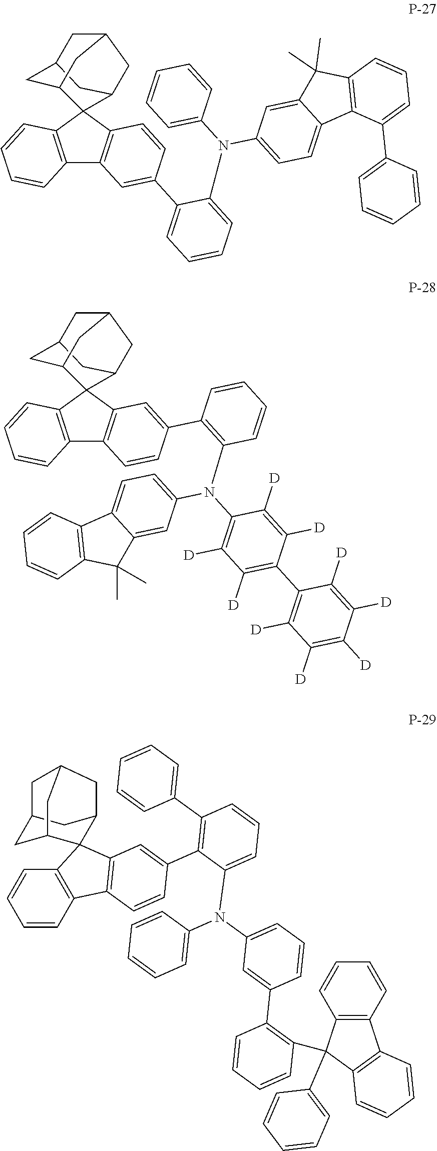 [SMILES: CC1(C)c2cc(N(c3ccccc3)c3ccccc3-c3ccc4c(c3)-c3ccccc3C43C4CC5CC(C4)CC3C5)ccc2-c2c(-c3ccccc3)cccc21 ; [2H]c1c([2H])c([2H])c(-c2c([2H])c([2H])c(N(c3ccc4c(c3)C(C)(C)c3ccccc3-4)c3ccccc3-c3ccc4c(c3)C3(c5ccccc5-4)C4CC5CC(C4)CC3C5)c([2H])c2[2H])c([2H])c1[2H] ; c1ccc(-c2cccc(N(c3ccccc3)c3cccc(-c4ccccc4C4(c5ccccc5)c5ccccc5-c5ccccc54)c3)c2-c2ccc3c(c2)C2(c4ccccc4-3)C3CC4CC(C3)CC2C4)cc1]

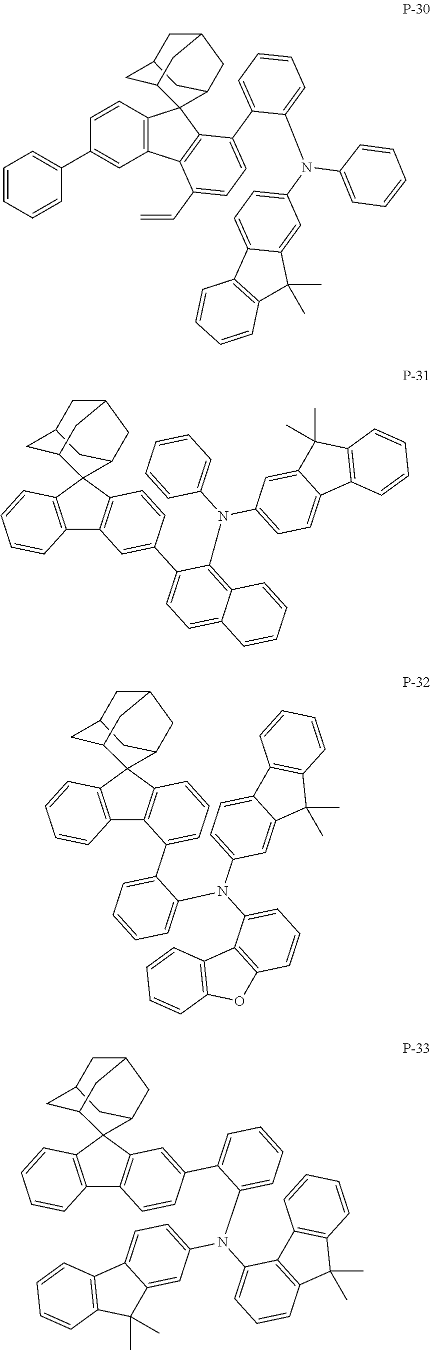 [SMILES: C=Cc1ccc(-c2ccccc2N(c2ccccc2)c2ccc3c(c2)C(C)(C)c2ccccc2-3)c2c1-c1cc(-c3ccccc3)ccc1C21C2CC3CC(C2)CC1C3 ; CC1(C)c2ccccc2-c2ccc(N(c3ccccc3)c3c(-c4ccc5c(c4)-c4ccccc4C54C5CC6CC(C5)CC4C6)ccc4ccccc34)cc21 ; CC1(C)c2ccccc2-c2ccc(N(c3ccccc3-c3cccc4c3-c3ccccc3C43C4CC5CC(C4)CC3C5)c3cccc4oc5ccccc5c34)cc21 ; CC1(C)c2ccccc2-c2ccc(N(c3ccccc3-c3ccc4c(c3)C3(c5ccccc5-4)C4CC5CC(C4)CC3C5)c3cccc4c3-c3ccccc3C4(C)C)cc21]

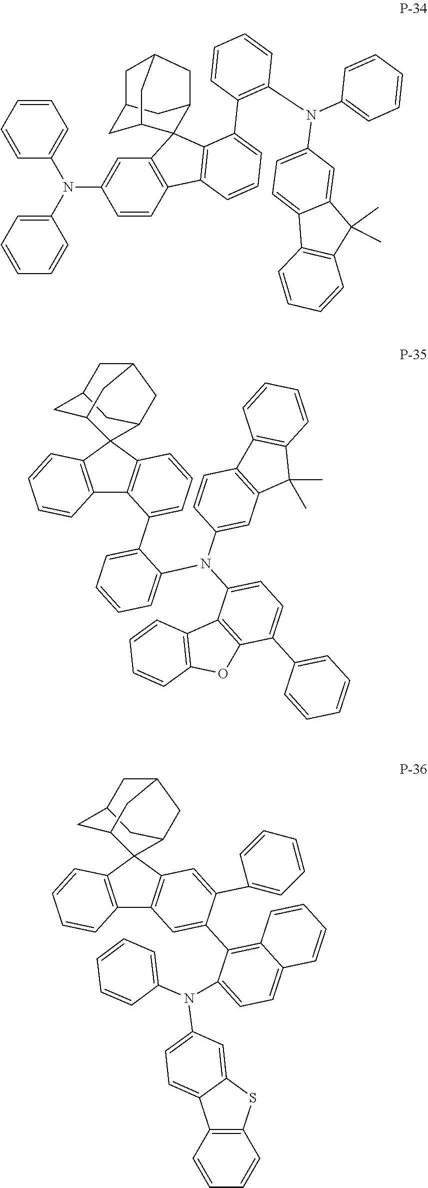 [SMILES: CC1(C)c2ccccc2-c2ccc(N(c3ccccc3)c3ccccc3-c3cccc4c3C3(c5cc(N(c6ccccc6)c6ccccc6)ccc5-4)C4CC5CC(C4)CC3C5)cc21 ; CC1(C)c2ccccc2-c2ccc(N(c3ccccc3-c3cccc4c3-c3ccccc3C43C4CC5CC(C4)CC3C5)c3ccc(-c4ccccc4)c4oc5ccccc5c34)cc21 ; c1ccc(-c2cc3c(cc2-c2c(N(c4ccccc4)c4ccc5c(c4)sc4ccccc45)ccc4ccccc24)-c2ccccc2C32C3CC4CC(C3)CC2C4)cc1]

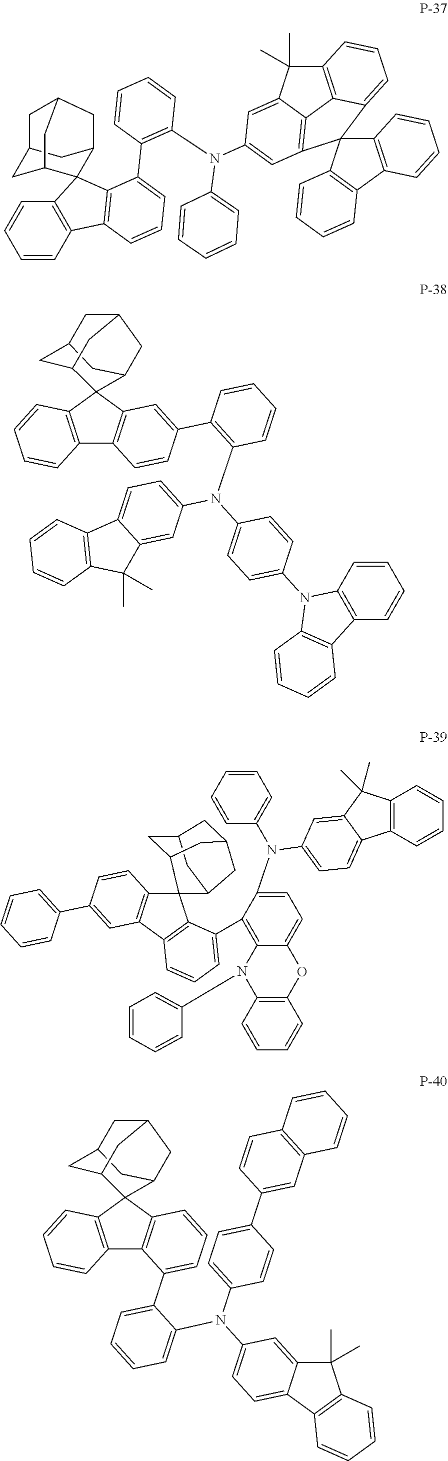 [SMILES: CC1(C)c2cccc3c2-c2c1cc(N(c1ccccc1)c1ccccc1-c1cccc4c1C1(c5ccccc5-4)C4CC5CC(C4)CC1C5)cc2C31c2ccccc2-c2ccccc21 ; CC1(C)c2ccccc2-c2ccc(N(c3ccc(-n4c5ccccc5c5ccccc54)cc3)c3ccccc3-c3ccc4c(c3)C3(c5ccccc5-4)C4CC5CC(C4)CC3C5)cc21 ; CC1(C)c2ccccc2-c2ccc(N(c3ccccc3)c3ccc4c(c3-c3cccc5c3C3(c6ccc(-c7ccccc7)cc6-5)C5CC6CC(C5)CC3C6)N(c3ccccc3)c3ccccc3O4)cc21 ; CC1(C)c2ccccc2-c2ccc(N(c3ccc(-c4ccc5ccccc5c4)cc3)c3ccccc3-c3cccc4c3-c3ccccc3C43C4CC5CC(C4)CC3C5)cc21]

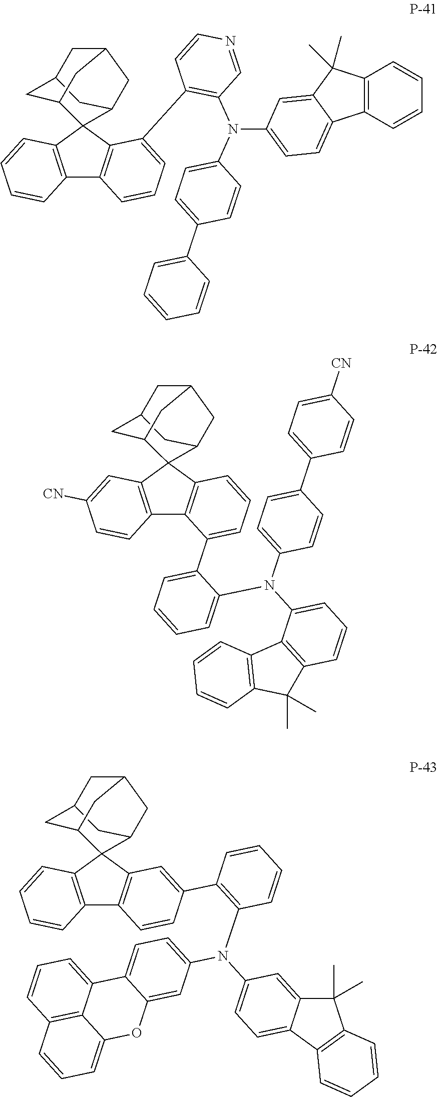

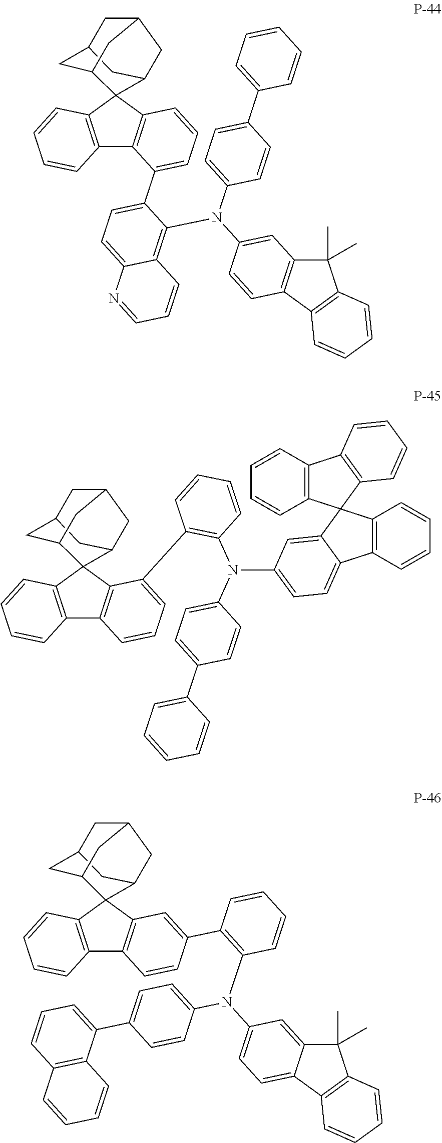 [SMILES: CC1(C)c2ccccc2-c2ccc(N(c3ccc(-c4ccccc4)cc3)c3c(-c4cccc5c4-c4ccccc4C54C5CC6CC(C5)CC4C6)ccc4ncccc34)cc21 ; c1ccc(-c2ccc(N(c3ccc4c(c3)C3(c5ccccc5-c5ccccc53)c3ccccc3-4)c3ccccc3-c3cccc4c3C3(c5ccccc5-4)C4CC5CC(C4)CC3C5)cc2)cc1 ; CC1(C)c2ccccc2-c2ccc(N(c3ccc(-c4cccc5ccccc45)cc3)c3ccccc3-c3ccc4c(c3)C3(c5ccccc5-4)C4CC5CC(C4)CC3C5)cc21]

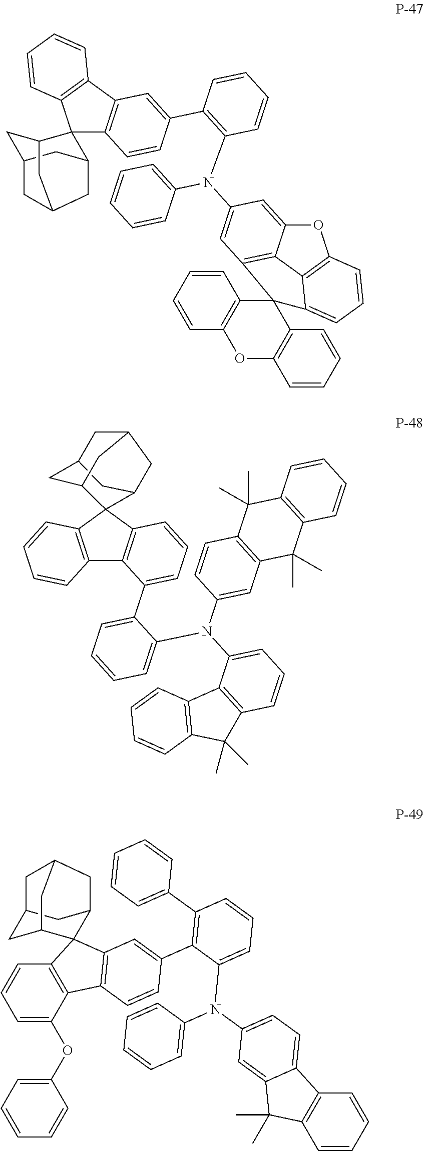 [SMILES: c1ccc(N(c2cc3c4c(c2)oc2cccc(c24)C32c3ccccc3Oc3ccccc32)c2ccccc2-c2ccc3c(c2)-c2ccccc2C32C3CC4CC(C3)CC2C4)cc1 ; CC1(C)c2ccccc2C(C)(C)c2cc(N(c3ccccc3-c3cccc4c3-c3ccccc3C43C4CC5CC(C4)CC3C5)c3cccc4c3-c3ccccc3C4(C)C)ccc21 ; CC1(C)c2ccccc2-c2ccc(N(c3ccccc3)c3cccc(-c4ccccc4)c3-c3ccc4c(c3)C3(c5cccc(Oc6ccccc6)c5-4)C4CC5CC(C4)CC3C5)cc21]

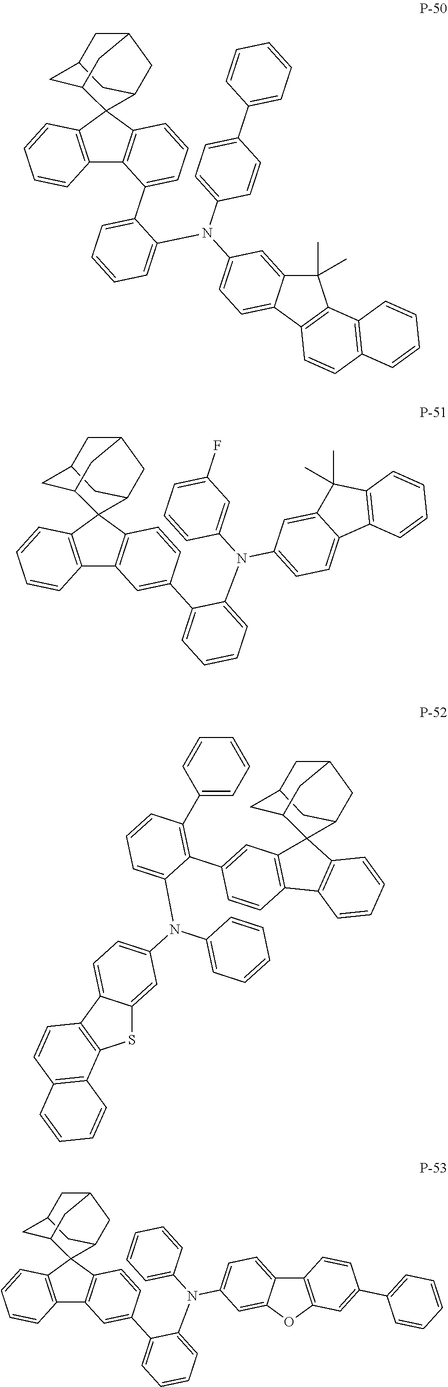 [SMILES: CC1(C)c2cc(N(c3ccc(-c4ccccc4)cc3)c3ccccc3-c3cccc4c3-c3ccccc3C43C4CC5CC(C4)CC3C5)ccc2-c2ccc3ccccc3c21 ; CC1(C)c2ccccc2-c2ccc(N(c3cccc(F)c3)c3ccccc3-c3ccc4c(c3)-c3ccccc3C43C4CC5CC(C4)CC3C5)cc21 ; c1ccc(-c2cccc(N(c3ccccc3)c3ccc4c(c3)sc3c5ccccc5ccc43)c2-c2ccc3c(c2)C2(c4ccccc4-3)C3CC4CC(C3)CC2C4)cc1 ; c1ccc(-c2ccc3c(c2)oc2cc(N(c4ccccc4)c4ccccc4-c4ccc5c(c4)-c4ccccc4C54C5CC6CC(C5)CC4C6)ccc23)cc1]

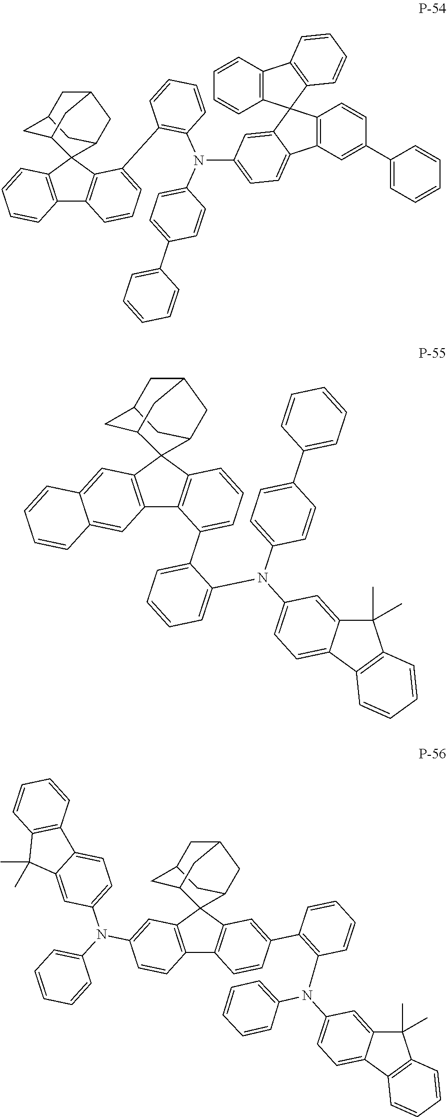 [SMILES: c1ccc(-c2ccc(N(c3ccc4c(c3)C3(c5ccccc5-c5ccccc53)c3ccc(-c5ccccc5)cc3-4)c3ccccc3-c3cccc4c3C3(c5ccccc5-4)C4CC5CC(C4)CC3C5)cc2)cc1 ; CC1(C)c2ccccc2-c2ccc(N(c3ccc(-c4ccccc4)cc3)c3ccccc3-c3cccc4c3-c3cc5ccccc5cc3C43C4CC5CC(C4)CC3C5)cc21 ; CC1(C)c2ccccc2-c2ccc(N(c3ccccc3)c3ccc4c(c3)C3(c5cc(-c6ccccc6N(c6ccccc6)c6ccc7c(c6)C(C)(C)c6ccccc6-7)ccc5-4)C4CC5CC(C4)CC3C5)cc21]

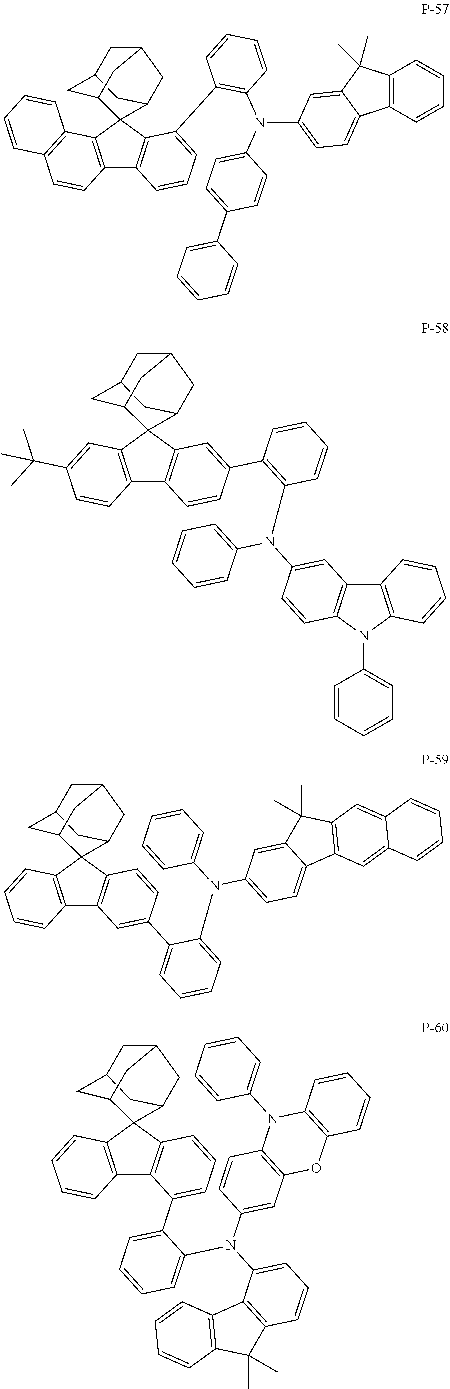 [SMILES: CC1(C)c2ccccc2-c2ccc(N(c3ccc(-c4ccccc4)cc3)c3ccccc3-c3cccc4c3C3(c5c-4ccc4ccccc54)C4CC5CC(C4)CC3C5)cc21 ; CC(C)(C)c1ccc2c(c1)C1(c3cc(-c4ccccc4N(c4ccccc4)c4ccc5c(c4)c4ccccc4n5-c4ccccc4)ccc3-2)C2CC3CC(C2)CC1C3 ; CC1(C)c2cc(N(c3ccccc3)c3ccccc3-c3ccc4c(c3)-c3ccccc3C43C4CC5CC(C4)CC3C5)ccc2-c2cc3ccccc3cc21 ; CC1(C)c2ccccc2-c2c(N(c3ccc4c(c3)Oc3ccccc3N4c3ccccc3)c3ccccc3-c3cccc4c3-c3ccccc3C43C4CC5CC(C4)CC3C5)cccc21]

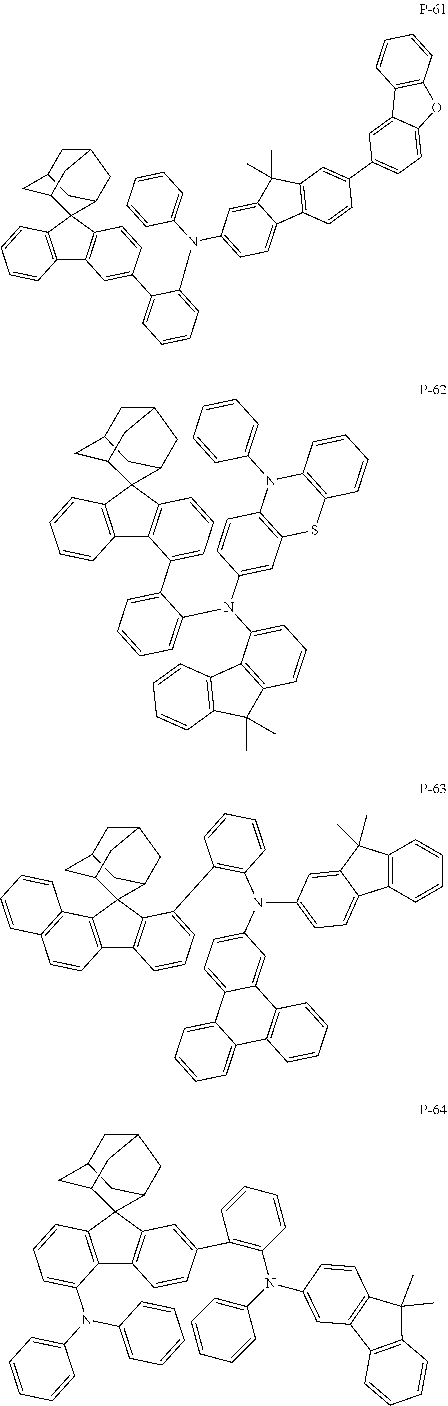 [SMILES: CC1(C)c2cc(-c3ccc4oc5ccccc5c4c3)ccc2-c2ccc(N(c3ccccc3)c3ccccc3-c3ccc4c(c3)-c3ccccc3C43C4CC5CC(C4)CC3C5)cc21 ; CC1(C)c2ccccc2-c2c(N(c3ccc4c(c3)Sc3ccccc3N4c3ccccc3)c3ccccc3-c3cccc4c3-c3ccccc3C43C4CC5CC(C4)CC3C5)cccc21 ; CC1(C)c2ccccc2-c2ccc(N(c3ccc4c5ccccc5c5ccccc5c4c3)c3ccccc3-c3cccc4c3C3(c5c-4ccc4ccccc54)C4CC5CC(C4)CC3C5)cc21 ; CC1(C)c2ccccc2-c2cc(N(c3ccccc3)c3ccccc3-c3ccc4c(c3)C3(c5cccc(N(c6ccccc6)c6ccccc6)c5-4)C4CC5CC(C4)CC3C5)ccc21]

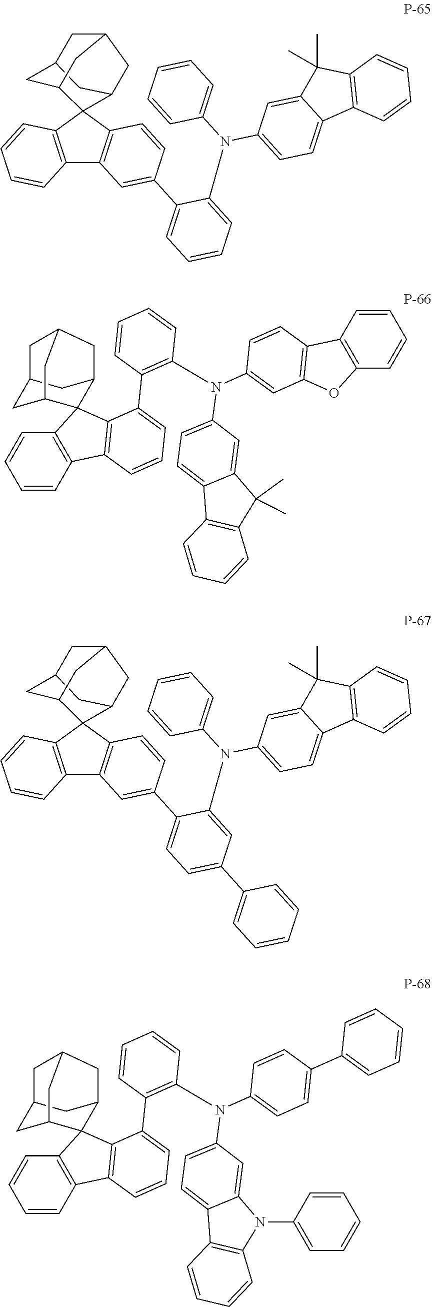 [SMILES: CC1(C)c2ccccc2-c2ccc(N(c3ccccc3)c3ccccc3-c3ccc4c(c3)-c3ccccc3C43C4CC5CC(C4)CC3C5)cc21 ; CC1(C)c2ccccc2-c2ccc(N(c3ccc4c(c3)oc3ccccc34)c3ccccc3-c3cccc4c3C3(c5ccccc5-4)C4CC5CC(C4)CC3C5)cc21 ; CC1(C)c2ccccc2-c2ccc(N(c3ccccc3)c3cc(-c4ccccc4)ccc3-c3ccc4c(c3)-c3ccccc3C43C4CC5CC(C4)CC3C5)cc21 ; c1ccc(-c2ccc(N(c3ccc4c5ccccc5n(-c5ccccc5)c4c3)c3ccccc3-c3cccc4c3C3(c5ccccc5-4)C4CC5CC(C4)CC3C5)cc2)cc1]

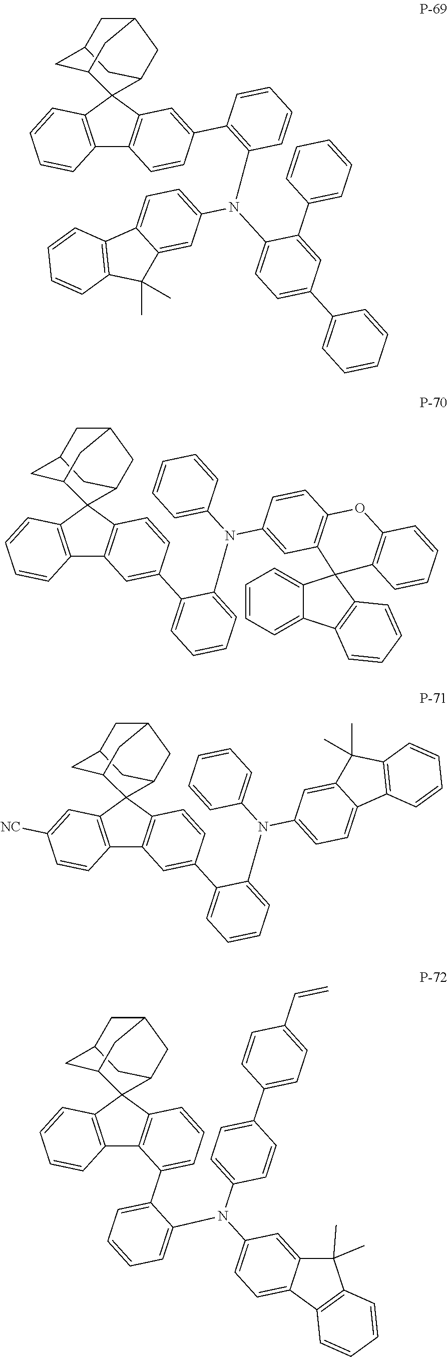 [SMILES: CC1(C)c2ccccc2-c2ccc(N(c3ccccc3-c3ccc4c(c3)C3(c5ccccc5-4)C4CC5CC(C4)CC3C5)c3ccc(-c4ccccc4)cc3-c3ccccc3)cc21 ; c1ccc(N(c2ccc3c(c2)C2(c4ccccc4O3)c3ccccc3-c3ccccc32)c2ccccc2-c2ccc3c(c2)-c2ccccc2C32C3CC4CC(C3)CC2C4)cc1 ; CC1(C)c2ccccc2-c2ccc(N(c3ccccc3)c3ccccc3-c3ccc4c(c3)-c3ccc(C#N)cc3C43C4CC5CC(C4)CC3C5)cc21 ; C=Cc1ccc(-c2ccc(N(c3ccc4c(c3)C(C)(C)c3ccccc3-4)c3ccccc3-c3cccc4c3-c3ccccc3C43C4CC5CC(C4)CC3C5)cc2)cc1]

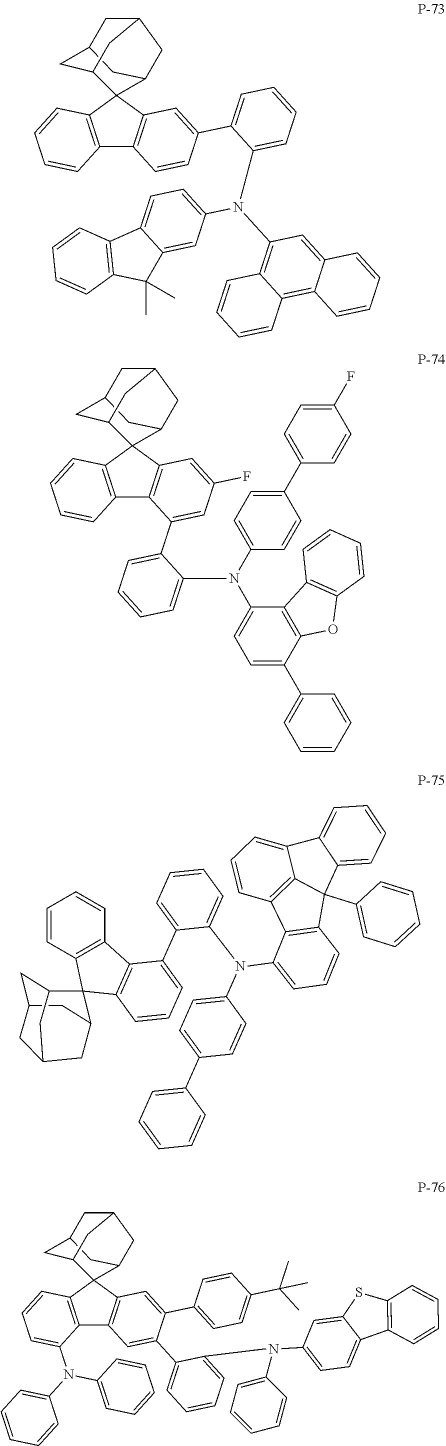 [SMILES: CC1(C)c2ccccc2-c2ccc(N(c3ccccc3-c3ccc4c(c3)C3(c5ccccc5-4)C4CC5CC(C4)CC3C5)c3cc4ccccc4c4ccccc34)cc21 ; Fc1ccc(-c2ccc(N(c3ccccc3-c3cc(F)cc4c3-c3ccccc3C43C4CC5CC(C4)CC3C5)c3ccc(-c4ccccc4)c4oc5ccccc5c34)cc2)cc1 ; c1ccc(-c2ccc(N(c3ccccc3-c3cccc4c3-c3ccccc3C43C4CC5CC(C4)CC3C5)c3cccc4c3-c3cccc5c3C4(c3ccccc3)c3ccccc3-5)cc2)cc1 ; CC(C)(C)c1ccc(-c2cc3c(cc2-c2ccccc2N(c2ccccc2)c2ccc4c(c2)sc2ccccc24)-c2c(N(c4ccccc4)c4ccccc4)cccc2C32C3CC4CC(C3)CC2C4)cc1]

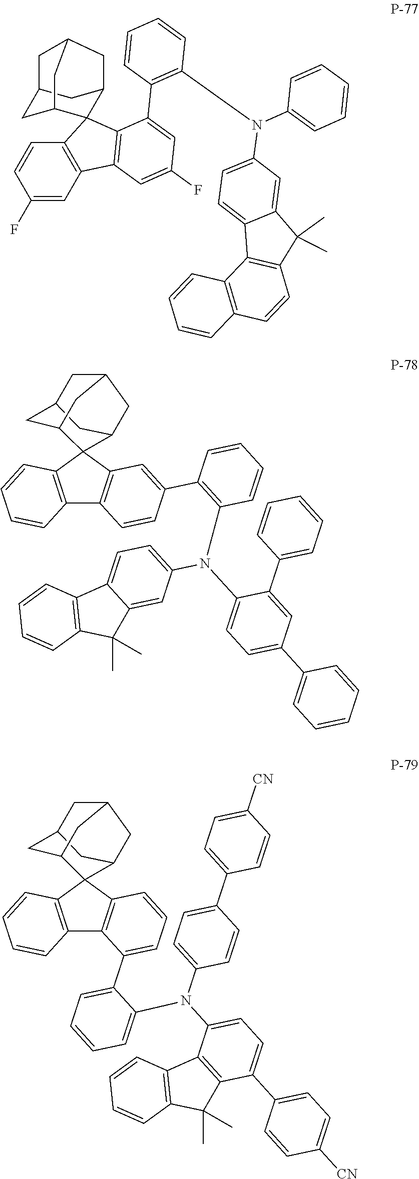 [SMILES: CC1(C)c2cc(N(c3ccccc3)c3ccccc3-c3cc(F)cc4c3C3(c5ccc(F)cc5-4)C4CC5CC(C4)CC3C5)ccc2-c2c1ccc1ccccc21 ; CC1(C)c2ccccc2-c2ccc(N(c3ccccc3-c3ccc4c(c3)C3(c5ccccc5-4)C4CC5CC(C4)CC3C5)c3ccc(-c4ccccc4)cc3-c3ccccc3)cc21 ; CC1(C)c2ccccc2-c2c(N(c3ccc(-c4ccc(C#N)cc4)cc3)c3ccccc3-c3cccc4c3-c3ccccc3C43C4CC5CC(C4)CC3C5)ccc(-c3ccc(C#N)cc3)c21]

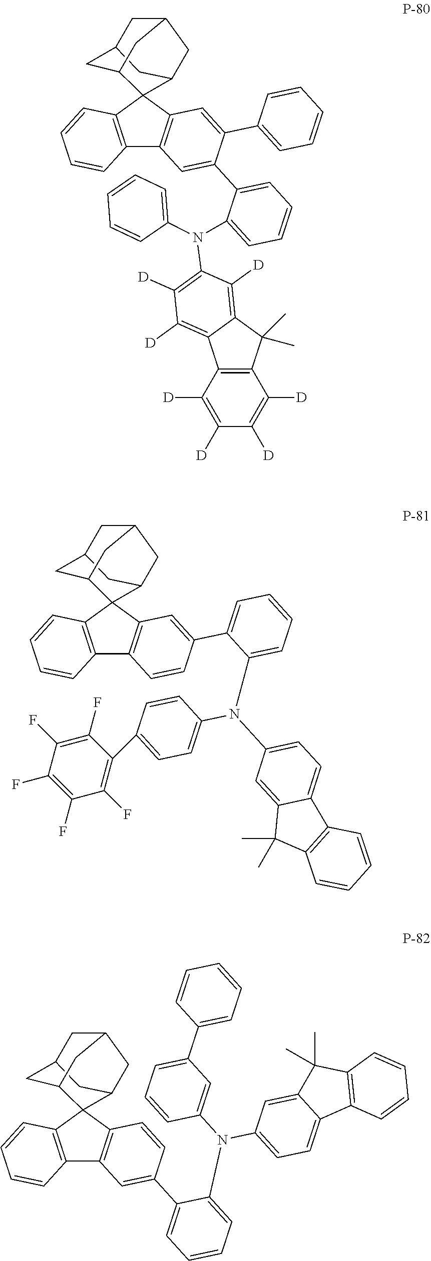 [SMILES: [2H]c1c([2H])c([2H])c2c(c1[2H])-c1c([2H])c([2H])c(N(c3ccccc3)c3ccccc3-c3cc4c(cc3-c3ccccc3)C3(c5ccccc5-4)C4CC5CC(C4)CC3C5)c([2H])c1C2(C)C ; CC1(C)c2ccccc2-c2ccc(N(c3ccc(-c4c(F)c(F)c(F)c(F)c4F)cc3)c3ccccc3-c3ccc4c(c3)C3(c5ccccc5-4)C4CC5CC(C4)CC3C5)cc21 ; CC1(C)c2ccccc2-c2ccc(N(c3cccc(-c4ccccc4)c3)c3ccccc3-c3ccc4c(c3)-c3ccccc3C43C4CC5CC(C4)CC3C5)cc21]

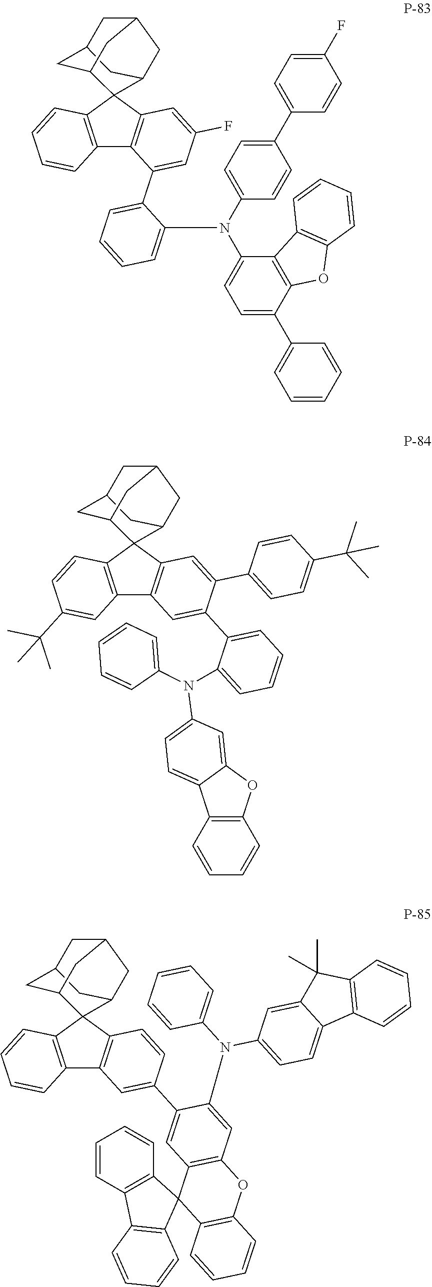 [SMILES: Fc1ccc(-c2ccc(N(c3ccccc3-c3cc(F)cc4c3-c3ccccc3C43C4CC5CC(C4)CC3C5)c3ccc(-c4ccccc4)c4oc5ccccc5c34)cc2)cc1 ; CC(C)(C)c1ccc(-c2cc3c(cc2-c2ccccc2N(c2ccccc2)c2ccc4c(c2)oc2ccccc24)-c2cc(C(C)(C)C)ccc2C32C3CC4CC(C3)CC2C4)cc1 ; CC1(C)c2ccccc2-c2ccc(N(c3ccccc3)c3cc4c(cc3-c3ccc5c(c3)-c3ccccc3C53C5CC6CC(C5)CC3C6)C3(c5ccccc5O4)c4ccccc4-c4ccccc43)cc21]

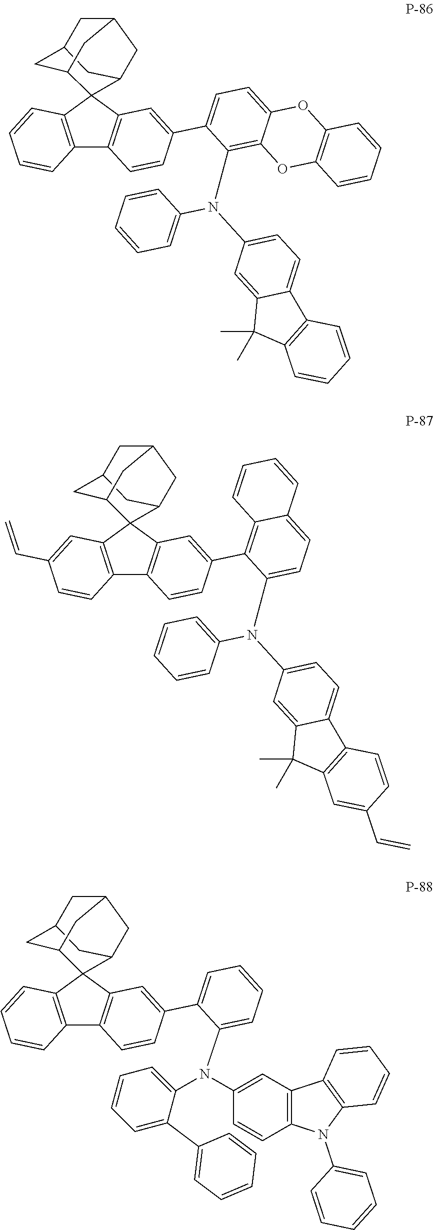 [SMILES: CC1(C)c2ccccc2-c2ccc(N(c3ccccc3)c3c(-c4ccc5c(c4)C4(c6ccccc6-5)C5CC6CC(C5)CC4C6)ccc4c3Oc3ccccc3O4)cc21 ; C=Cc1ccc2c(c1)C(C)(C)c1cc(N(c3ccccc3)c3ccc4ccccc4c3-c3ccc4c(c3)C3(c5cc(C=C)ccc5-4)C4CC5CC(C4)CC3C5)ccc1-2 ; c1ccc(-c2ccccc2N(c2ccc3c(c2)c2ccccc2n3-c2ccccc2)c2ccccc2-c2ccc3c(c2)C2(c4ccccc4-3)C3CC4CC(C3)CC2C4)cc1]

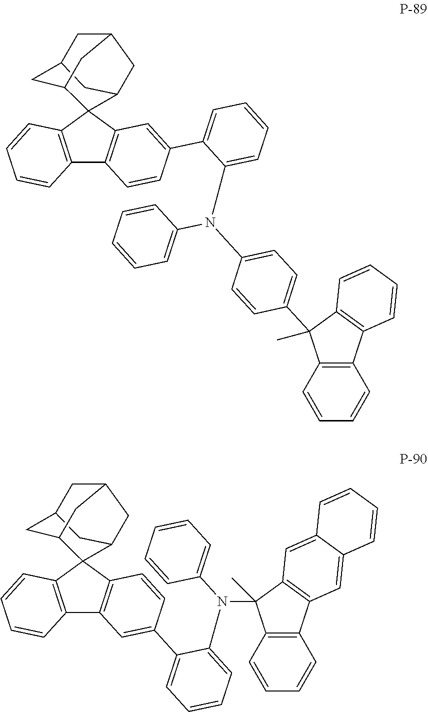 [SMILES: CC1(c2ccc(N(c3ccccc3)c3ccccc3-c3ccc4c(c3)C3(c5ccccc5-4)C4CC5CC(C4)CC3C5)cc2)c2ccccc2-c2ccccc21 ; CC1(N(c2ccccc2)c2ccccc2-c2ccc3c(c2)-c2ccccc2C32C3CC4CC(C3)CC2C4)c2ccccc2-c2cc3ccccc3cc21]

As other embodiment of the present disclosure, the present disclosure provides an organic electric device comprising: a first electrode; a second electrode; and an organic material layer formed between the first electrode and the second electrode, wherein the organic material layer contains the compounds represented by Chemical Formula 1 alone or in mixtures thereof.

As another embodiment of the present disclosure, the present disclosure provides an organic electric device comprising: a first electrode; a second electrode; an organic material layer formed between the first electrode and the second electrode; and a capping layer, wherein the capping layer is formed on one surface of both surfaces of the first electrode and the second electrode that is not in contact with the organic material layer, and the organic material layer or the capping layer contains the compounds represented by Chemical Formula 1 alone or in mixtures thereof.

The organic material layer includes at least one of a hole injection layer, a hole transport layer, a light emitting auxiliary layer, a light emitting layer, an electron transport auxiliary layer, an electron transport layer, and an electron injection layer. That is, at least one of the hole injection layer, the hole transport layer, the light emitting auxiliary layer, the light emitting layer, the electron transport auxiliary layer, the electron transport layer, and the electron injection layer included in the organic material layer may contain the compounds represented by Chemical Formula 1.

Preferably, the organic material layer includes at least one of the hole transport layer, the light emitting auxiliary layer, and the light emitting layer. That is, the compounds may be contained in at least one of the hole transport layer, the light emitting auxiliary layer, and the light emitting layer.

The organic material layer may contain two or more stacks including a hole transport layer, a light emitting layer, and an electron transport layer sequentially formed on the anode.

Preferably, the organic material layer further includes a charge generation layer formed between the two or more stacks.

As another embodiment of the present disclosure, the present disclosure provide an electronic device comprising a display device including an organic electric device comprising the compounds represented by Chemical Formula 1 above and a controller for driving the display device.

In an embodiment of the present disclosure, the compounds of Chemical Formula 1 above may be contained alone, the compounds may be contained in combinations of two or more different compounds, or the compounds may be contained in combinations of two or more other compounds.

Hereinafter, Synthesis Examples of the compounds represented by Chemical Formula 1 according to the present disclosure and Manufacturing Examples of the organic electric device will be described in detail with reference to Examples, but the present disclosure is not limited to the Examples below.

### Synthesis Example

The final product represented by Chemical Formula 1 above according to the present disclosure may be synthesized by reacting Sub 1 and Sub 2 as shown in Reaction Scheme 1 below, but is not limited thereto.

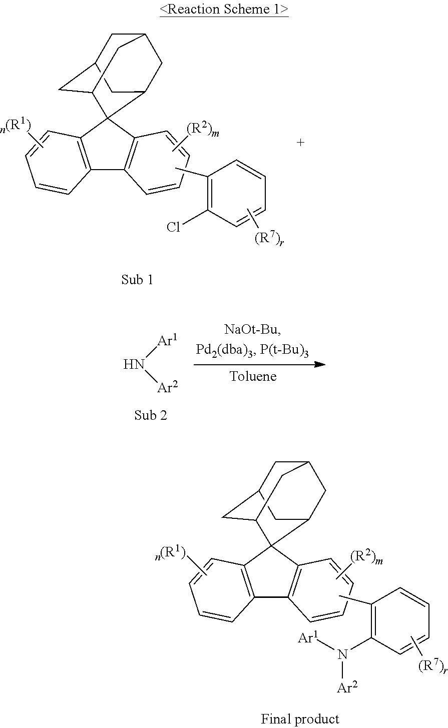

In Reaction Scheme 1,

R¹, R², R⁷, Ar¹, Ar², n, m, and r are the same as defined in Chemical Formula 1.

### I. Synthesis Example of Sub 1

Sub 1 of Reaction Scheme 1 above may be synthesized by Reaction Scheme 2 below, but is not limited thereto.

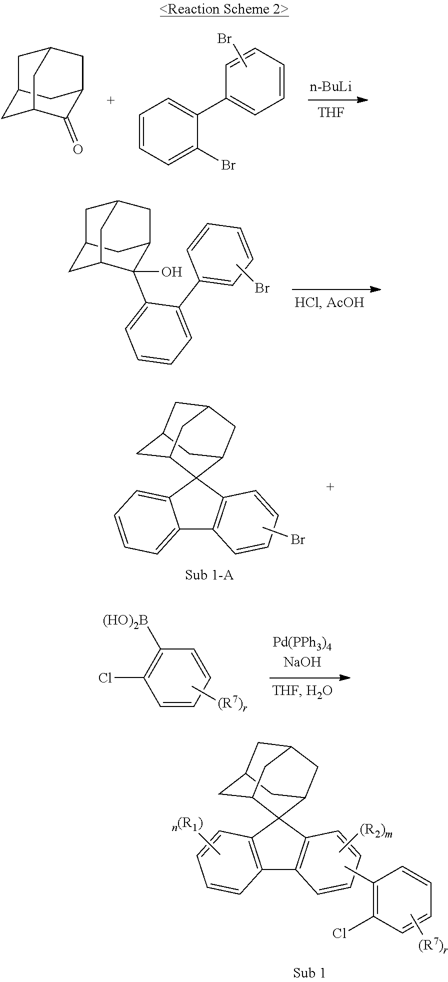

### Synthesis Example of Sub 1-A-1

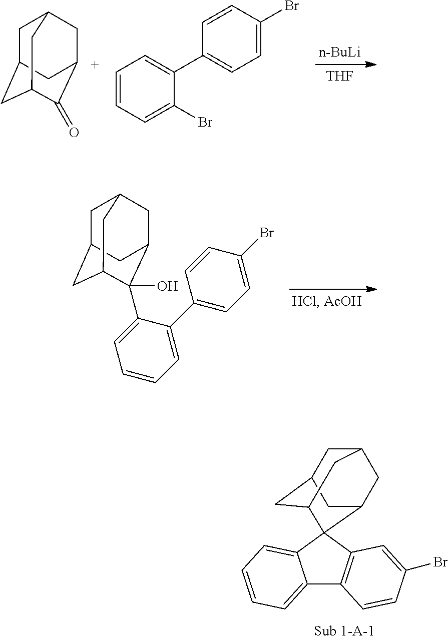

535 ml of THF and 50.00 g (160.25 mmol) of 2,2′-dibromo-1,1′-biphenyl were injected into a dried 2000 ml round bottom flask under a nitrogen atmosphere, and the mixture was cooled to −78° C. After that, 64.1 ml of n-BuLi was slowly added dropwise to the reaction container. 24.07 g (160.25 mmol) of adamantan-2-one was dissolved in 150 ml of THF, and then slowly added dropwise to the cooled reaction solution. The reaction solution was slowly heated to room temperature, and then reacted at room temperature for 3 hours. After the reaction was completed, the reaction solution was quenched with an 1N HCl aqueous solution and extracted with ethyl acetate. The extracted organic layer was dried over MgSO₄, filtered, and then concentrated. 500 ml of acetic acid and 100 ml of an HCl aqueous solution (35%) were injected into the concentrated mixture, and the mixture was refluxed and stirred for 6 hours. After completion of the reaction, the reaction solution was cooled to room temperature, and then the solid was filtered. The filtered solid was subjected to silica gel column chromatography to obtain 47.47 g (yield: 81.1%) of a product.

### Sub 1-1 Synthesis Example

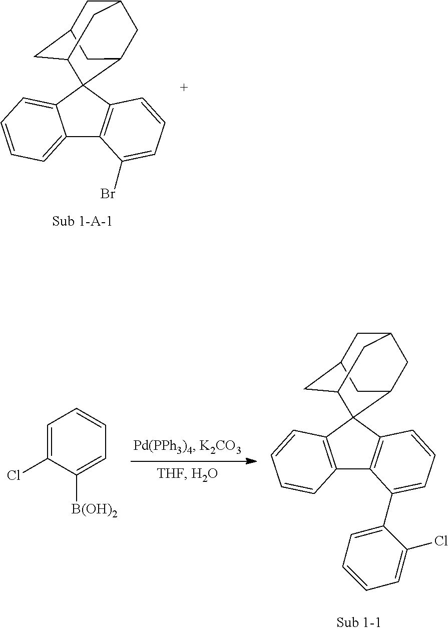 [SMILES: Brc1cccc2c1-c1ccccc1C21C2CC3CC(C2)CC1C3 ; OB(O)c1ccccc1Cl ; Clc1ccccc1-c1cccc2c1-c1ccccc1C21C2CC3CC(C2)CC1C3]

20.21 g (55.32 mmol) of Sub 1-A-1 obtained in the above synthesis, 9.08 g (58.09 mmol) of (2-chlorophenyl)boronic acid, 1.92 g (1.66 mmol) of Pd(PPh₃)₄, and 185 ml of THF were injected into a 500 ml round bottom flask, 22.94 g (165.97 mmol) of K₂CO₃ was dissolved in 45 ml of water and added thereto, and then the mixture was refluxed and stirred for 10 hours. After completion of the reaction, extraction was performed with ethyl acetate and water, and the organic layer was dried over MgSO₄, filtered, and then concentrated. The produced compound was subjected to silica gel column chromatography to obtain 20.13 g (yield: 91.7%) of a product.

### Sub 1-19 Synthesis Example

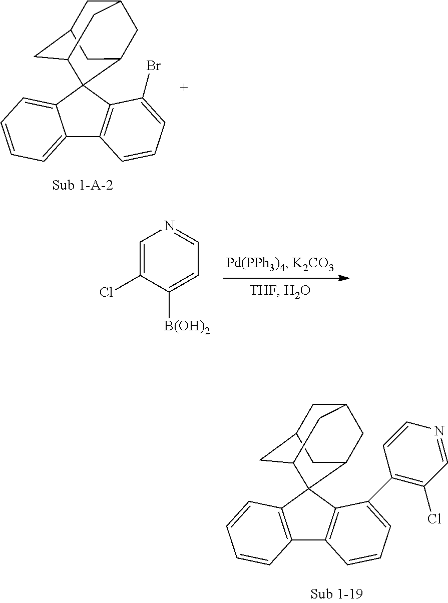 [SMILES: Brc1cccc2c1C1(c3ccccc3-2)C2CC3CC(C2)CC1C3 ; OB(O)c1ccncc1Cl ; Clc1cnccc1-c1cccc2c1C1(c3ccccc3-2)C2CC3CC(C2)CC1C3]

16.74 g (45.82 mmol) of Sub 1-A-2 obtained by performing synthesis in the same manner as in the synthesis of Sub 1-A-1 above, 7.57 g (48.12 mmol) of (3-chloropyridin-4-yl)boronic acid, 1.59 g (1.37 mmol) of Pd(PPh₃)₄, and 155 ml of THF were injected into a 500 ml round bottom flask, and 19.00 g (137.47 mmol) of K₂CO₃ was dissolved in 40 ml of water and added thereto, and then the mixture was refluxed and stirred for 10 hours. After completion of the reaction, extraction was performed with ethyl acetate and water, and the organic layer was dried over MgSO₄, filtered, and then concentrated. The produced compound was subjected to silica gel column chromatography to obtain 14.88 g (yield: 81.6%) of a product.

### Sub 1-20 Synthesis Example

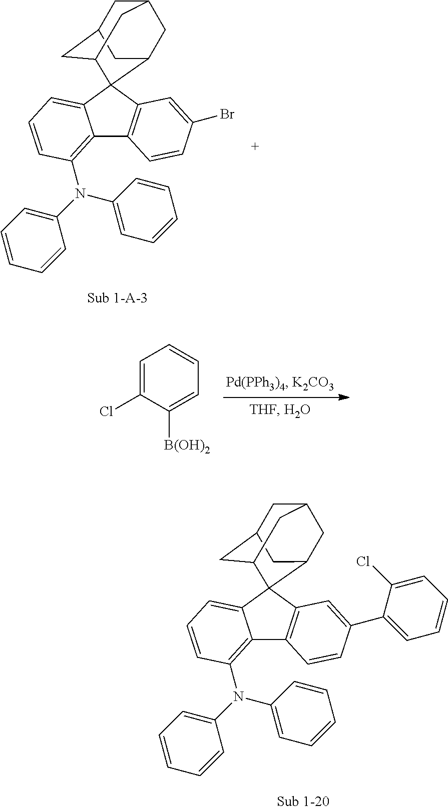 [SMILES: Brc1ccc2c(c1)C1(c3cccc(N(c4ccccc4)c4ccccc4)c3-2)C2CC3CC(C2)CC1C3 ; OB(O)c1ccccc1Cl ; Clc1ccccc1-c1ccc2c(c1)C1(c3cccc(N(c4ccccc4)c4ccccc4)c3-2)C2CC3CC(C2)CC1C3]

21.40 g (40.19 mmol) of Sub 1-A-3 obtained by performing synthesis in the same manner as in the synthesis of Sub 1-A-1 above, 6.60 g (42.19 mmol) of (2-chlorophenyl)boronic acid, 1.39 g (1.12 mmol) of Pd(PPh₃)₄, and 135 ml of THF were injected into a 500 ml round bottom flask, and 16.66 g (120.56 mmol) of K₂CO₃ was dissolved in 40 ml of water and added thereto, and then the mixture was refluxed and stirred for 10 hours. After completion of the reaction, extraction was performed with ethyl acetate and water, and the organic layer was dried over MgSO₄, filtered, and then concentrated. The produced compound was subjected to silica gel column chromatography to obtain 18.00 g (yield: 79.4%) of a product.

### Sub 1-25 Synthesis Example

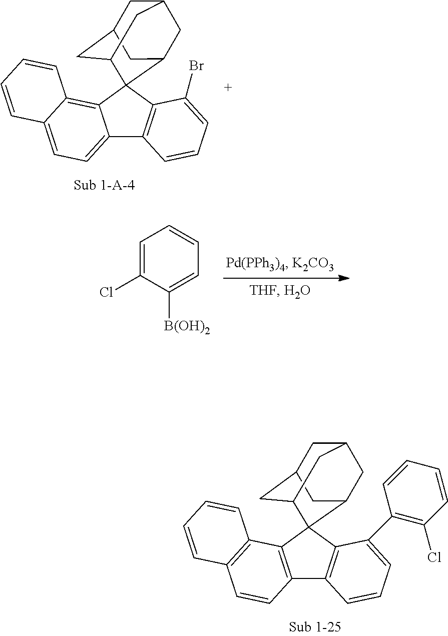 [SMILES: Brc1cccc2c1C1(c3c-2ccc2ccccc32)C2CC3CC(C2)CC1C3 ; OB(O)c1ccccc1Cl ; Clc1ccccc1-c1cccc2c1C1(c3c-2ccc2ccccc32)C2CC3CC(C2)CC1C3]

23.98 g (57.73 mmol) of Sub 1-A-4 obtained by performing synthesis in the same manner as in the synthesis of Sub 1-A-1 above, 9.48 g (60.62 mmol) of (2-chlorophenyl)boronic acid, 2.00 g (1.73 mmol) of Pd(PPh₃)₄, and 195 ml of THF were injected into a 500 ml round bottom flask, and 23.94 g (173.19 mmol) of K₂CO₃ was dissolved in 50 ml of water and added thereto, and then the mixture was refluxed and stirred for 10 hours. After completion of the reaction, extraction was performed with ethyl acetate and water, and the organic layer was dried over MgSO₄, filtered, and then concentrated. The produced compound was subjected to silica gel column chromatography to obtain 22.53 g (yield: 87.3%) of a product.

### Sub 1-40 Synthesis Example

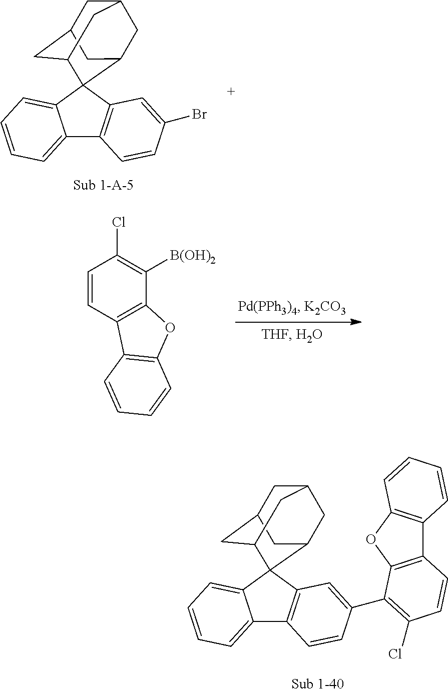 [SMILES: Brc1ccc2c(c1)C1(c3ccccc3-2)C2CC3CC(C2)CC1C3 ; OB(O)c1c(Cl)ccc2c1oc1ccccc12 ; Clc1ccc2c(oc3ccccc32)c1-c1ccc2c(c1)C1(c3ccccc3-2)C2CC3CC(C2)CC1C3]

19.76 g (54.09 mmol) of Sub 1-A-5 obtained by performing synthesis in the same manner as in the synthesis of Sub 1-A-1 above, 14.00 g (56.80 mmol) of (1-chlorodibenzo[b,d]furan-4-yl)boronic acid, 1.88 g (1.62 mmol) of Pd(PPh₃)₄, and 180 ml of THF were injected into a 500 ml round bottom flask, and 22.43 g (162.27 mmol) of K₂CO₃ was dissolved in 50 ml of water and added thereto, and then the mixture was refluxed and stirred for 10 hours. After completion of the reaction, extraction was performed with ethyl acetate and water, and the organic layer was dried over MgSO₄, filtered, and then concentrated. The produced compound was subjected to silica gel column chromatography to obtain 22.70 g (yield: 86.2%) of a product.

The compounds belonging to Sub 1 may be compounds as follows, but are not limited thereto.

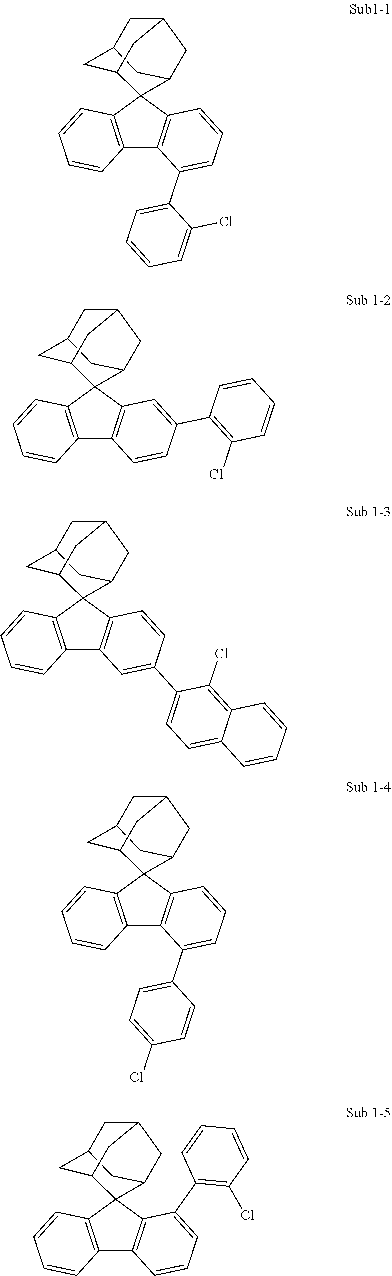

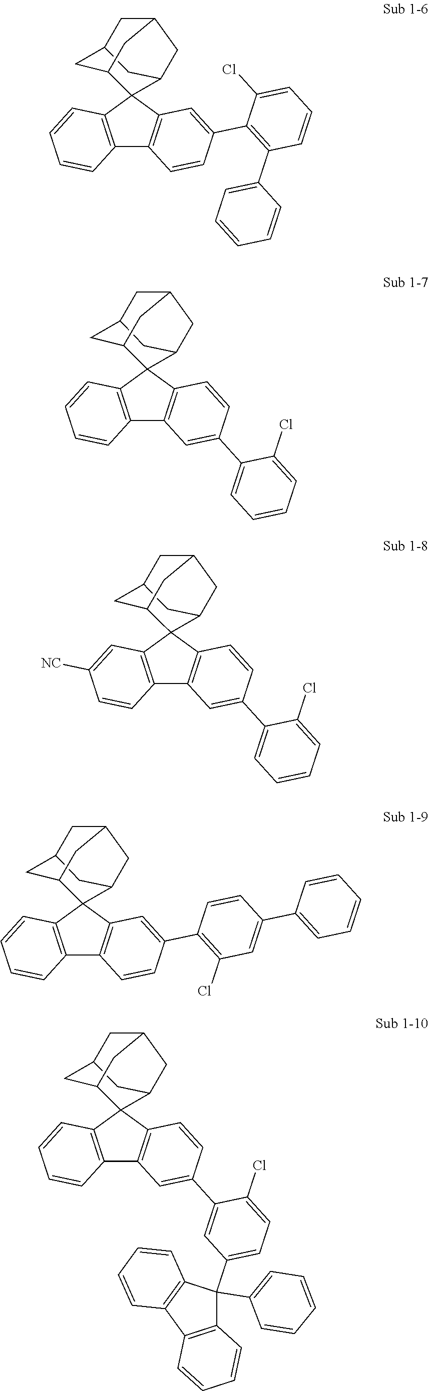 [SMILES: Clc1cccc(-c2ccccc2)c1-c1ccc2c(c1)C1(c3ccccc3-2)C2CC3CC(C2)CC1C3 ; Clc1ccccc1-c1ccc2c(c1)-c1ccccc1C21C2CC3CC(C2)CC1C3 ; N#Cc1ccc2c(c1)C1(c3ccc(-c4ccccc4Cl)cc3-2)C2CC3CC(C2)CC1C3 ; Clc1cc(-c2ccccc2)ccc1-c1ccc2c(c1)C1(c3ccccc3-2)C2CC3CC(C2)CC1C3 ; Clc1ccc(C2(c3ccccc3)c3ccccc3-c3ccccc32)cc1-c1ccc2c(c1)-c1ccccc1C21C2CC3CC(C2)CC1C3]

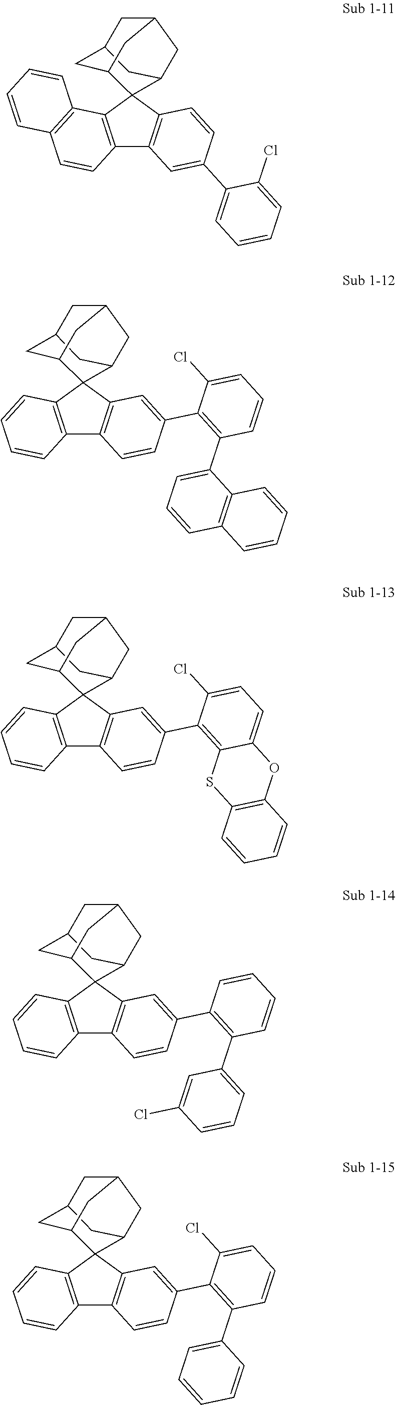 [SMILES: Clc1ccccc1-c1ccc2c(c1)-c1ccc3ccccc3c1C21C2CC3CC(C2)CC1C3 ; Clc1cccc(-c2cccc3ccccc23)c1-c1ccc2c(c1)C1(c3ccccc3-2)C2CC3CC(C2)CC1C3 ; Clc1ccc2c(c1-c1ccc3c(c1)C1(c4ccccc4-3)C3CC4CC(C3)CC1C4)Sc1ccccc1O2 ; Clc1cccc(-c2ccccc2-c2ccc3c(c2)C2(c4ccccc4-3)C3CC4CC(C3)CC2C4)c1 ; Clc1cccc(-c2ccccc2)c1-c1ccc2c(c1)C1(c3ccccc3-2)C2CC3CC(C2)CC1C3]

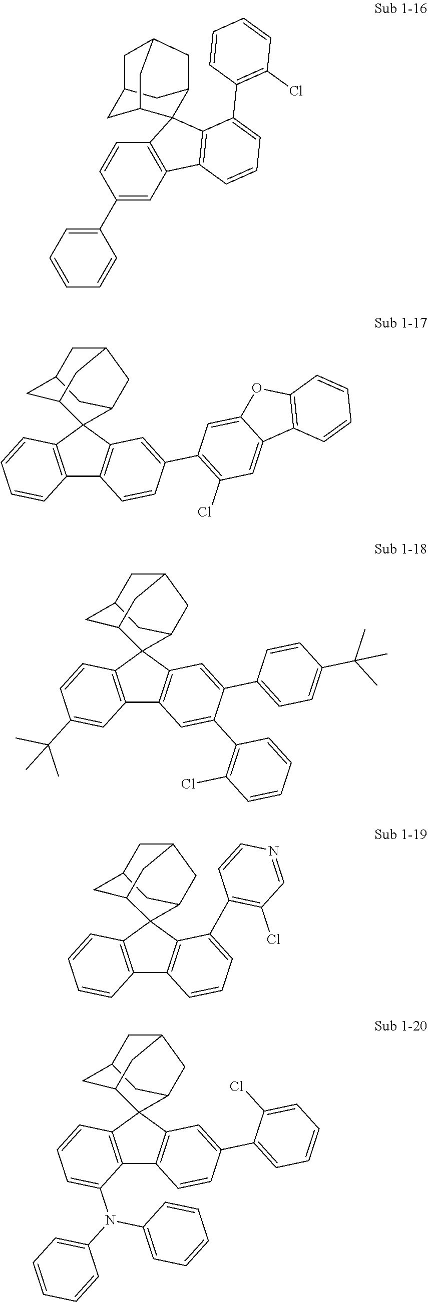

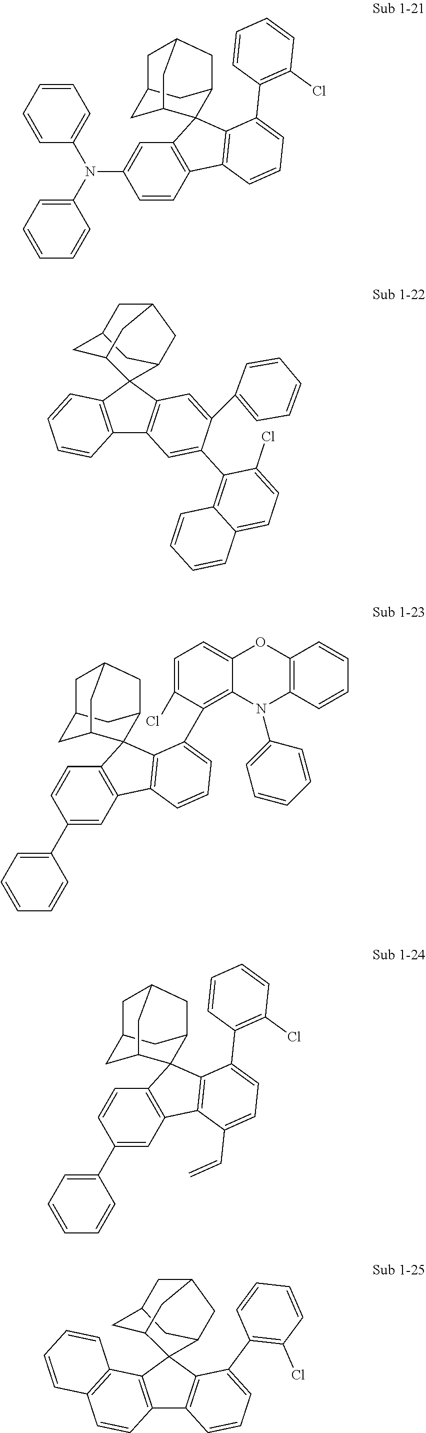 [SMILES: Clc1ccccc1-c1cccc2c1C1(c3cc(N(c4ccccc4)c4ccccc4)ccc3-2)C2CC3CC(C2)CC1C3 ; Clc1ccc2ccccc2c1-c1cc2c(cc1-c1ccccc1)C1(c3ccccc3-2)C2CC3CC(C2)CC1C3 ; Clc1ccc2c(c1-c1cccc3c1C1(c4ccc(-c5ccccc5)cc4-3)C3CC4CC(C3)CC1C4)N(c1ccccc1)c1ccccc1O2 ; C=Cc1ccc(-c2ccccc2Cl)c2c1-c1cc(-c3ccccc3)ccc1C21C2CC3CC(C2)CC1C3 ; Clc1ccccc1-c1cccc2c1C1(c3c-2ccc2ccccc32)C2CC3CC(C2)CC1C3]

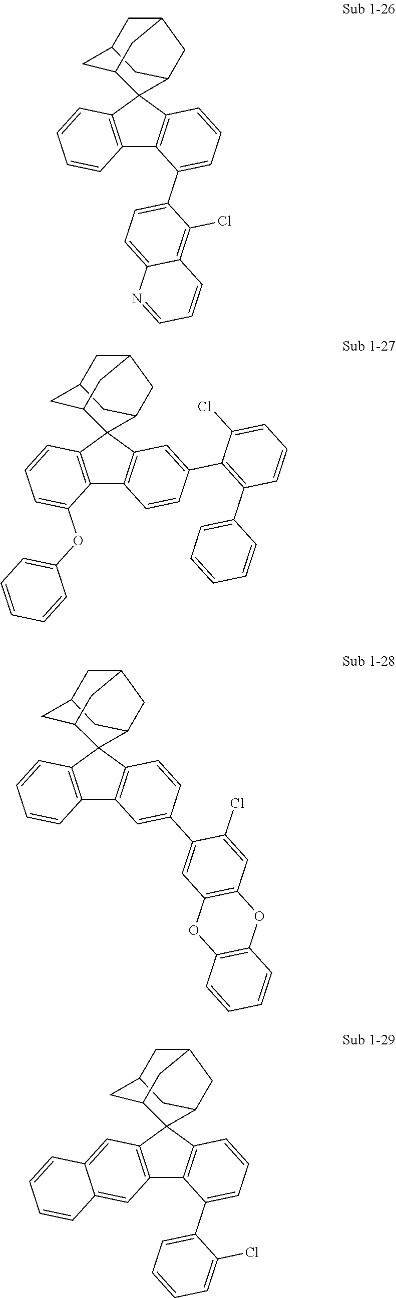

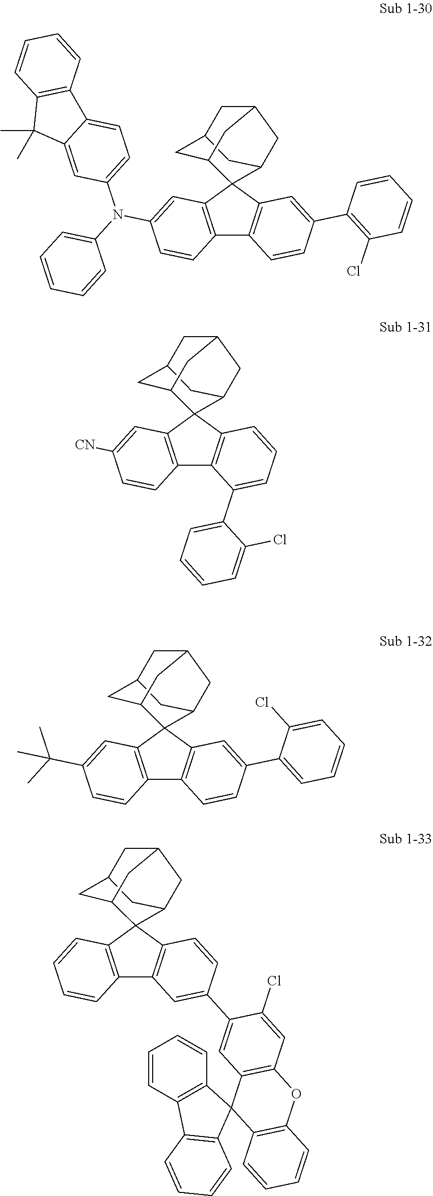

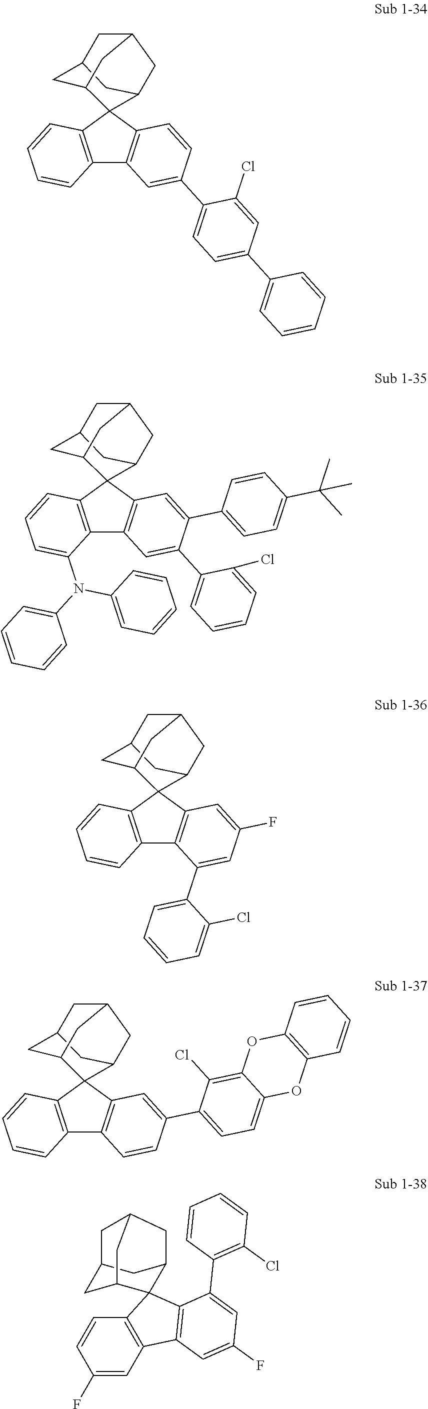 [SMILES: Clc1cc(-c2ccccc2)ccc1-c1ccc2c(c1)-c1ccccc1C21C2CC3CC(C2)CC1C3 ; CC(C)(C)c1ccc(-c2cc3c(cc2-c2ccccc2Cl)-c2c(N(c4ccccc4)c4ccccc4)cccc2C32C3CC4CC(C3)CC2C4)cc1 ; Fc1cc(-c2ccccc2Cl)c2c(c1)C1(c3ccccc3-2)C2CC3CC(C2)CC1C3 ; Clc1c(-c2ccc3c(c2)C2(c4ccccc4-3)C3CC4CC(C3)CC2C4)ccc2c1Oc1ccccc1O2 ; Fc1ccc2c(c1)-c1cc(F)cc(-c3ccccc3Cl)c1C21C2CC3CC(C2)CC1C3]

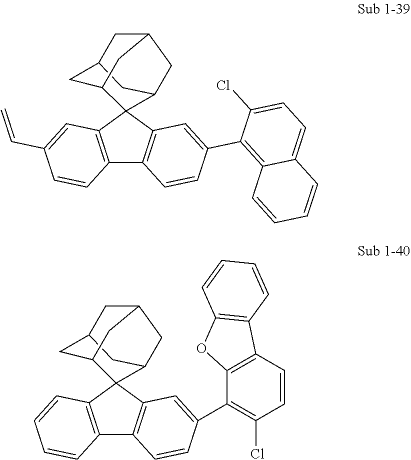

Table 1 below shows FD-MS values of the compounds belonging to Sub 1.

TABLE 1

| Compound | FD-MS | Compound | FD-MS |
|---|---|---|---|
| Sub 1-1 | m/z = 396.16(C₂₈H₂₅Cl = 396.96) | Sub 1-2 | m/z = 396.16(C₂₈H₂₅Cl = 396.96) |
| Sub 1-3 | m/z = 446.18(C₃₂H₂₇Cl = 447.02) | Sub 1-4 | m/z = 396.16(C₂₈H₂₅Cl = 396.96) |
| Sub 1-5 | m/z = 396.16(C₂₈H₂₅Cl = 396.96) | Sub 1-6 | m/z = 472.20(C₃₄H₂₉Cl = 473.06) |
| Sub 1-7 | m/z = 396.16(C₂₈H₂₅Cl = 396.96) | Sub 1-8 | m/z = 421.16(C₂₉H₂₄ClN = 421.97) |
| Sub 1-9 | m/z = 472.20(C₃₄H₂₉Cl = 473.06) | Sub 1-10 | m/z = 636.26(C₄₇H₃₇Cl = 637.26) |
| Sub 1-11 | m/z = 446.18(C₃₂H₂₇Cl = 447.02) | Sub 1-12 | m/z = 522.21(C₃₈H₃₁Cl = 523.12) |
| Sub 1-13 | m/z = 518.15(C₃₄H₂₇ClOS = 519.10) | Sub 1-14 | m/z = 472.20(C₃₄H₂₉Cl = 473.06) |
| Sub 1-15 | m/z = 472.20(C₃₄H₂₉Cl = 473.06) | Sub 1-16 | m/z = 472.20(C₃₄H₂₉Cl = 473.06) |
| Sub 1-17 | m/z = 486.18(C₃₄H₂₇ClO = 487.04) | Sub 1-18 | m/z = 486.18(C₃₄H₂₇ClO = 487.04) |
| Sub 1-19 | m/z = 397.16(C₂₇H₂₄ClN = 397.95) | Sub 1-20 | m/z = 563.24(C₄₀H₃₄ClN = 564.17) |
| Sub 1-21 | m/z = 563.24(C₄₀H₃₄ClN = 564.17) | Sub 1-22 | m/z = 522.21(C₃₈H₃₁Cl = 523.12) |
| Sub 1-23 | m/z = 654.25(C₄₆H₃₆ClNO = 653.25) | Sub 1-24 | m/z = 498.21(C₃₆H₃₁Cl = 499.09) |
| Sub 1-25 | m/z = 446.18(C₃₂H₂₇Cl = 447.02) | Sub 1-26 | m/z = 447.18(C₃₁H₂₆ClN = 448.01) |
| Sub 1-27 | m/z = 564.22(C₄₀H₃₃ClO = 565.15) | Sub 1-28 | m/z = 502.17(C₃₄H₂₇ClO₂ = 503.04) |
| Sub 1-29 | m/z = 446.18(C₃₂H₂₇Cl = 447.02) | Sub 1-30 | m/z = 679.30(C₄₉H₄₂ClN = 680.33) |
| Sub 1-31 | m/z = 421.16(C₂₉H₂₄ClN = 421.97) | Sub 1-32 | m/z = 452.23(C₃₂H₃₃Cl = 453.07) |
| Sub 1-33 | m/z = 650.24(C₄₇H₃₅ClO = 651.25) | Sub 1-34 | m/z = 472.20(C₃₄H₂₉Cl = 473.06) |
| Sub 1-35 | m/z = 695.33(C₅₀H₄₆ClN = 696.38) | Sub 1-36 | m/z = 414.16(C₂₈H₂₄ClF = 414.95) |
| Sub 1-37 | m/z = 502.17(C₃₄H₂₇ClO₂ = 503.04) | Sub 1-38 | m/z = 432.15(C₂₈H₂₃ClF₂ = 432.94) |
| Sub 1-39 | m/z = 472.20(C₃₄H₂₉Cl = 473.06) | Sub 1-40 | m/z = 584.32(C₄₂H₄₅Cl = 585.27) |

### II. Synthesis Example of Sub 2

Sub 2 of Reaction Scheme 1 above may be synthesized by the reaction route of Reaction Scheme 3 below, but is not limited thereto.

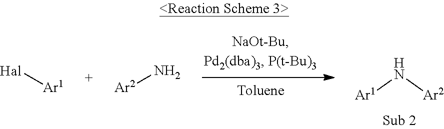

Meanwhile, the compounds belonging to Sub 2 may be compounds as follows, but are not limited thereto.

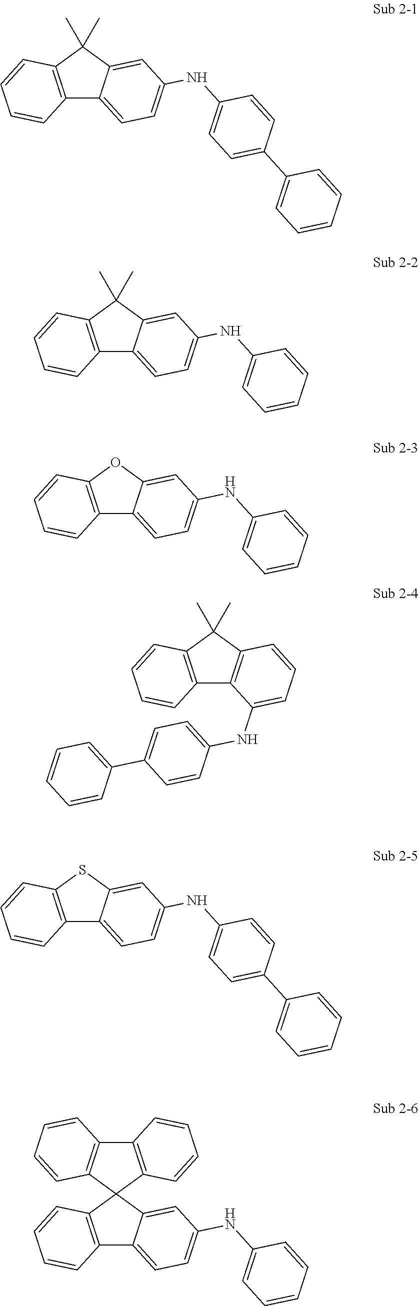

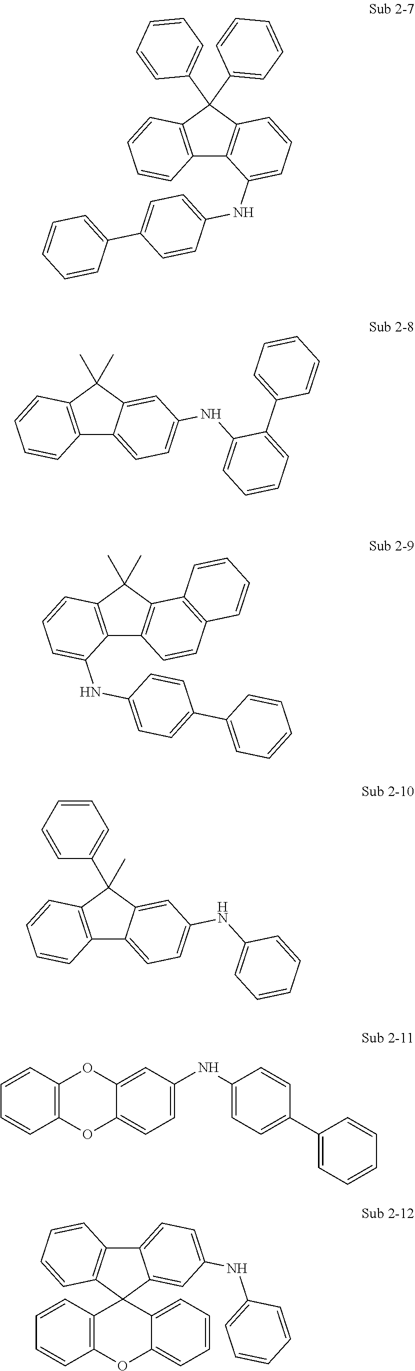 [SMILES: c1ccc(-c2ccc(Nc3cccc4c3-c3ccccc3C4(c3ccccc3)c3ccccc3)cc2)cc1 ; CC1(C)c2ccccc2-c2ccc(Nc3ccccc3-c3ccccc3)cc21 ; CC1(C)c2cccc(Nc3ccc(-c4ccccc4)cc3)c2-c2ccc3ccccc3c21 ; CC1(c2ccccc2)c2ccccc2-c2ccc(Nc3ccccc3)cc21 ; c1ccc(-c2ccc(Nc3ccc4c(c3)Oc3ccccc3O4)cc2)cc1 ; c1ccc(Nc2ccc3c(c2)C2(c4ccccc4Oc4ccccc42)c2ccccc2-3)cc1]

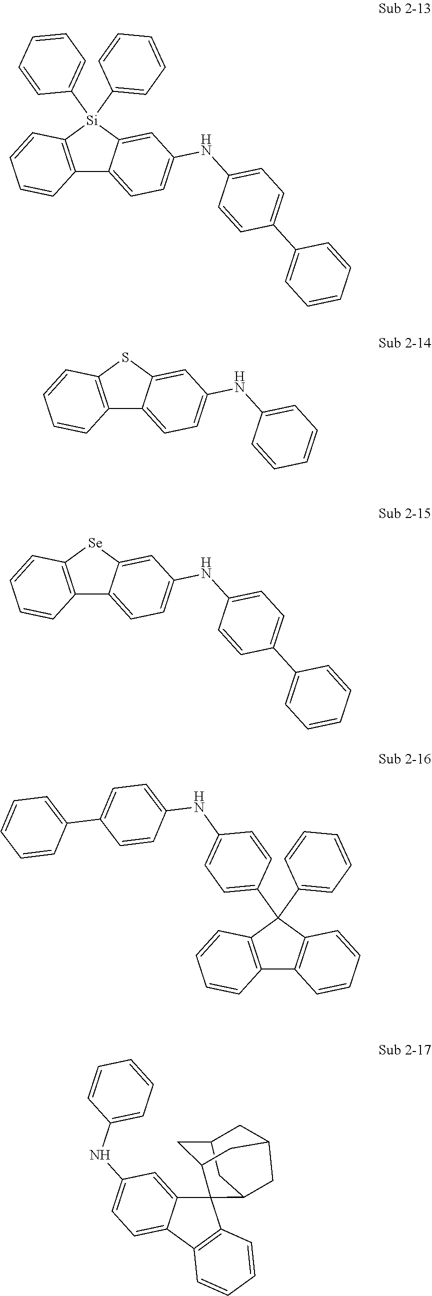 [SMILES: c1ccc(-c2ccc(Nc3ccc4c(c3)[Si](c3ccccc3)(c3ccccc3)c3ccccc3-4)cc2)cc1 ; c1ccc(Nc2ccc3c(c2)sc2ccccc23)cc1 ; c1ccc(-c2ccc(Nc3ccc4c(c3)[se]c3ccccc34)cc2)cc1 ; c1ccc(-c2ccc(Nc3ccc(C4(c5ccccc5)c5ccccc5-c5ccccc54)cc3)cc2)cc1 ; c1ccc(Nc2ccc3c(c2)C2(c4ccccc4-3)C3CC4CC(C3)CC2C4)cc1]

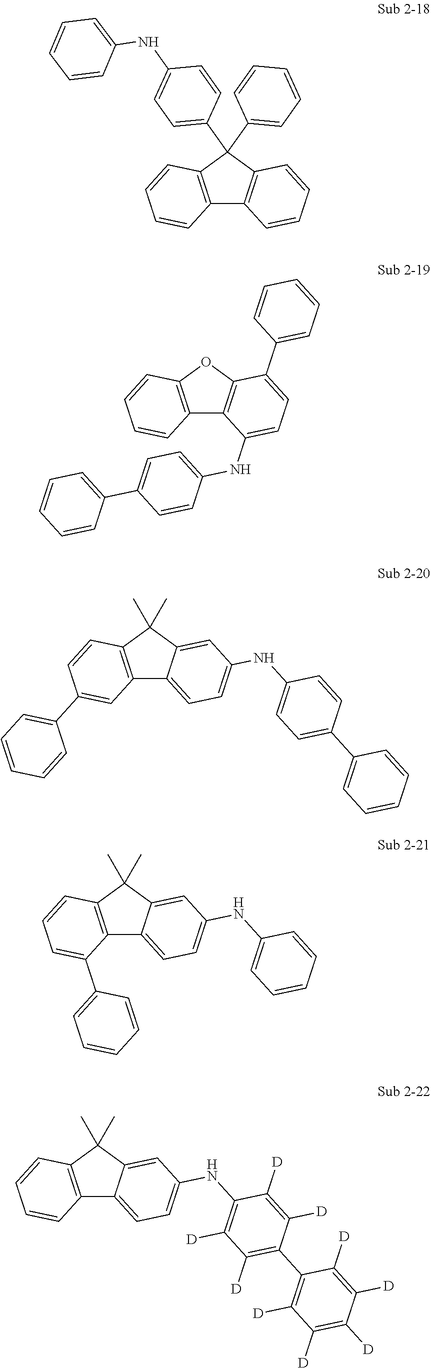 [SMILES: c1ccc(Nc2ccc(C3(c4ccccc4)c4ccccc4-c4ccccc43)cc2)cc1 ; c1ccc(-c2ccc(Nc3ccc(-c4ccccc4)c4oc5ccccc5c34)cc2)cc1 ; CC1(C)c2ccc(-c3ccccc3)cc2-c2ccc(Nc3ccc(-c4ccccc4)cc3)cc21 ; CC1(C)c2cc(Nc3ccccc3)ccc2-c2c(-c3ccccc3)cccc21 ; [2H]c1c([2H])c([2H])c(-c2c([2H])c([2H])c(Nc3ccc4c(c3)C(C)(C)c3ccccc3-4)c([2H])c2[2H])c([2H])c1[2H]]

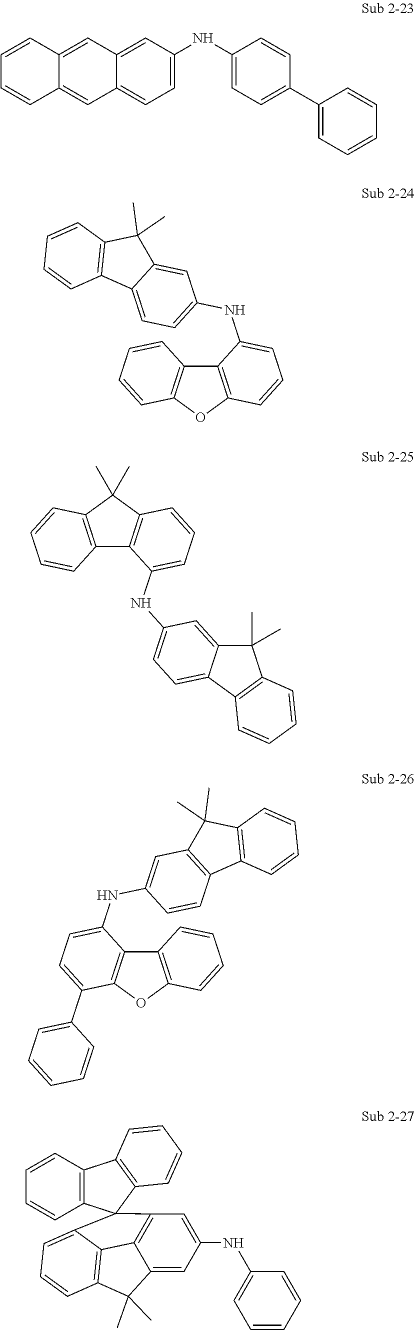 [SMILES: c1ccc(-c2ccc(Nc3ccc4cc5ccccc5cc4c3)cc2)cc1 ; CC1(C)c2ccccc2-c2ccc(Nc3cccc4oc5ccccc5c34)cc21 ; CC1(C)c2ccccc2-c2ccc(Nc3cccc4c3-c3ccccc3C4(C)C)cc21 ; CC1(C)c2ccccc2-c2ccc(Nc3ccc(-c4ccccc4)c4oc5ccccc5c34)cc21 ; CC1(C)c2cccc3c2-c2c1cc(Nc1ccccc1)cc2C31c2ccccc2-c2ccccc21]

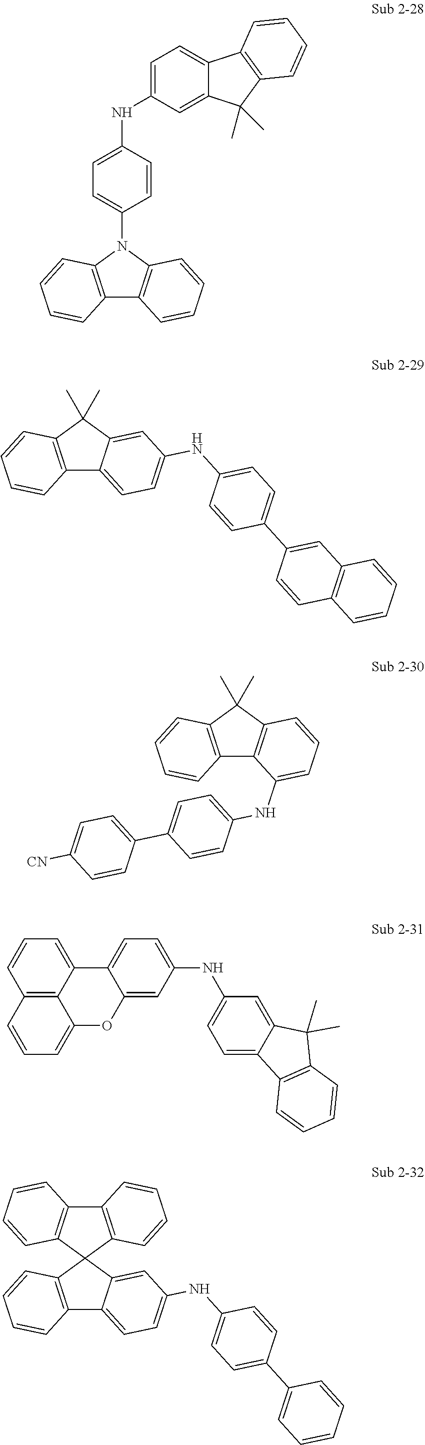

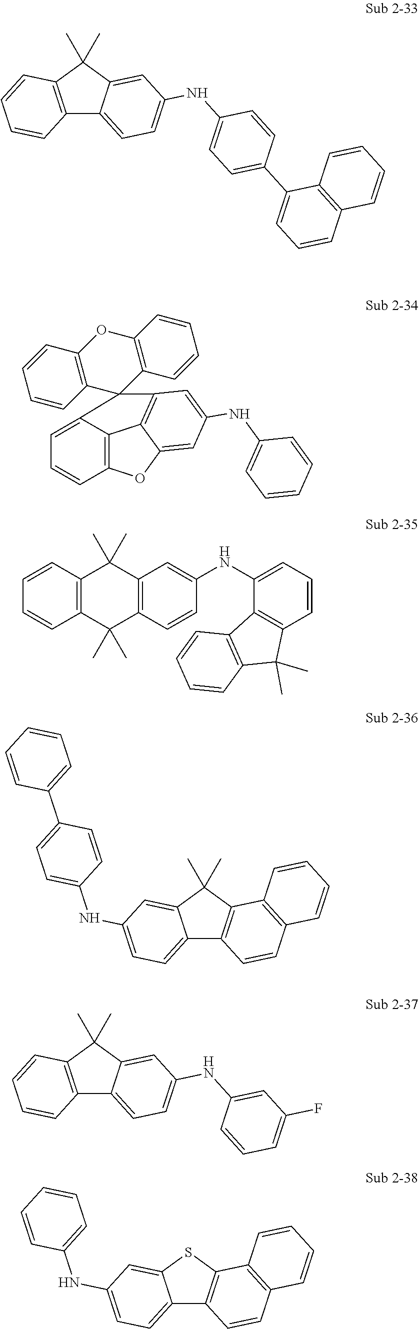 [SMILES: CC1(C)c2ccccc2-c2ccc(Nc3ccc(-c4cccc5ccccc45)cc3)cc21 ; c1ccc(Nc2cc3c4c(c2)oc2cccc(c24)C32c3ccccc3Oc3ccccc32)cc1 ; CC1(C)c2ccccc2C(C)(C)c2cc(Nc3cccc4c3-c3ccccc3C4(C)C)ccc21 ; CC1(C)c2cc(Nc3ccc(-c4ccccc4)cc3)ccc2-c2ccc3ccccc3c21 ; CC1(C)c2ccccc2-c2ccc(Nc3cccc(F)c3)cc21 ; c1ccc(Nc2ccc3c(c2)sc2c4ccccc4ccc32)cc1]

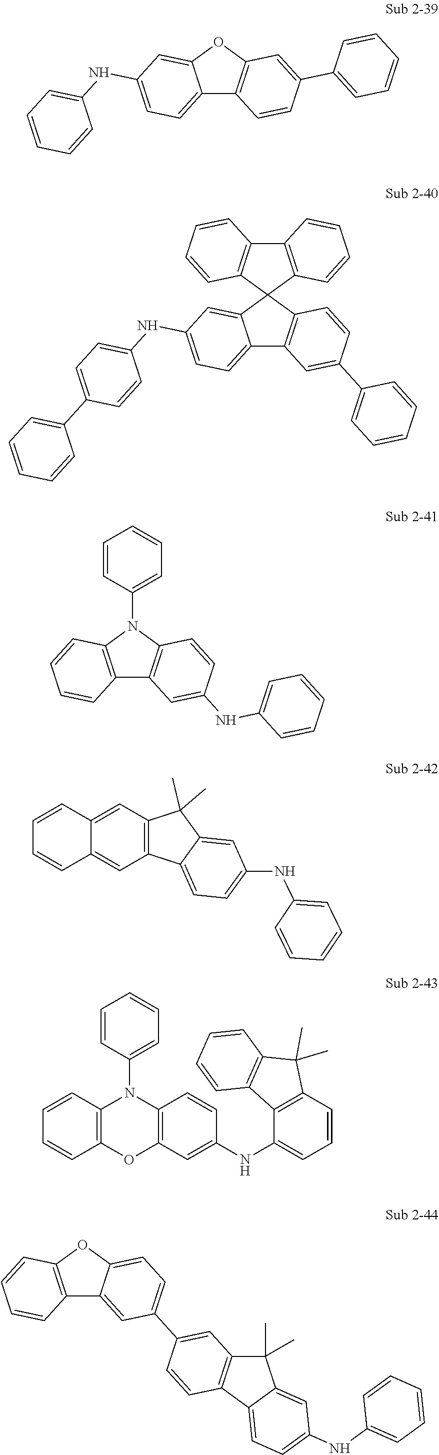 [SMILES: c1ccc(Nc2ccc3c(c2)oc2cc(-c4ccccc4)ccc23)cc1 ; c1ccc(-c2ccc(Nc3ccc4c(c3)C3(c5ccccc5-c5ccccc53)c3ccc(-c5ccccc5)cc3-4)cc2)cc1 ; c1ccc(Nc2ccc3c(c2)c2ccccc2n3-c2ccccc2)cc1 ; CC1(C)c2cc(Nc3ccccc3)ccc2-c2cc3ccccc3cc21 ; CC1(C)c2ccccc2-c2c(Nc3ccc4c(c3)Oc3ccccc3N4c3ccccc3)cccc21 ; CC1(C)c2cc(Nc3ccccc3)ccc2-c2ccc(-c3ccc4oc5ccccc5c4c3)cc21]

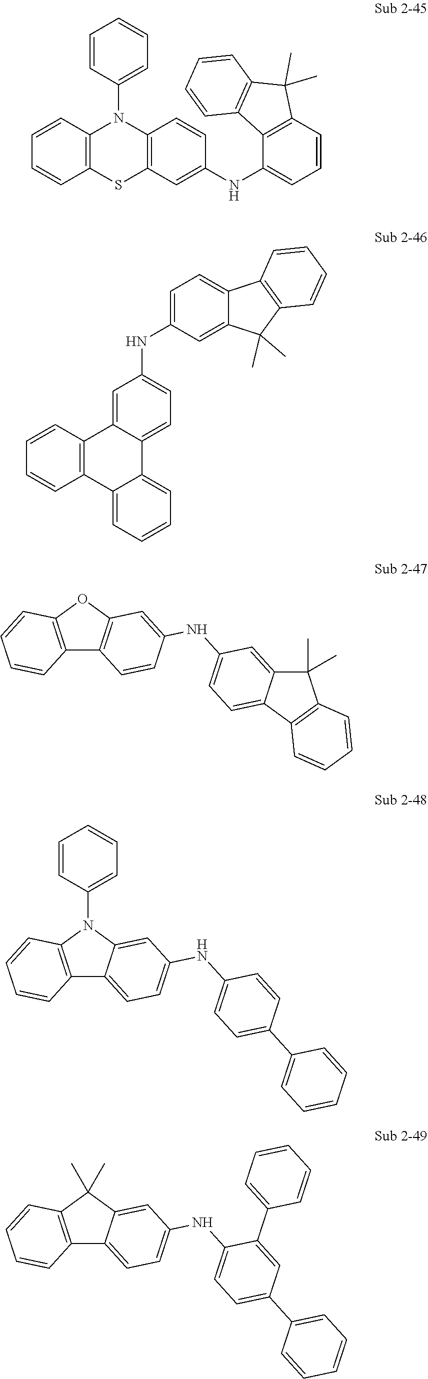

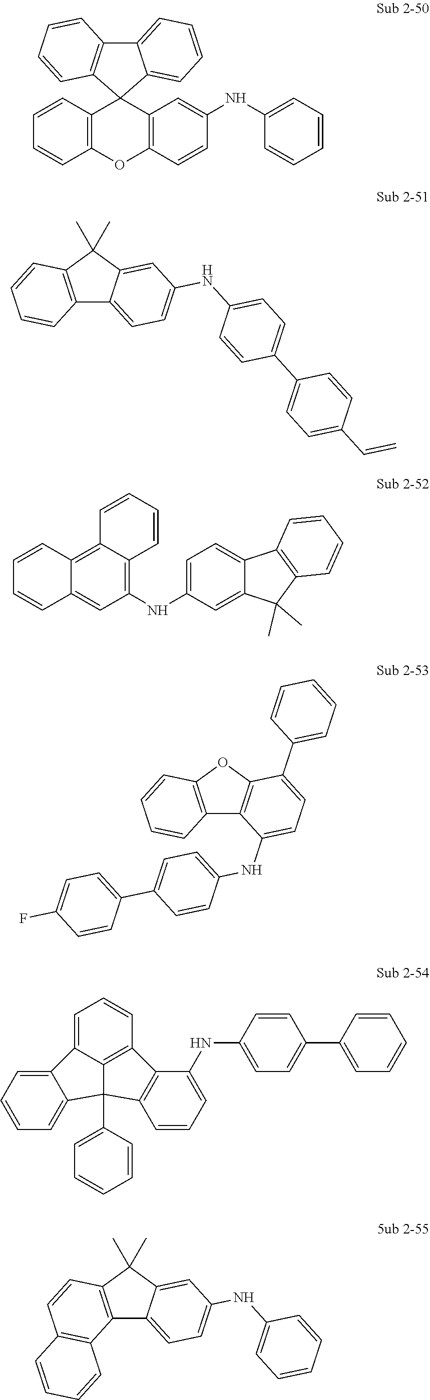

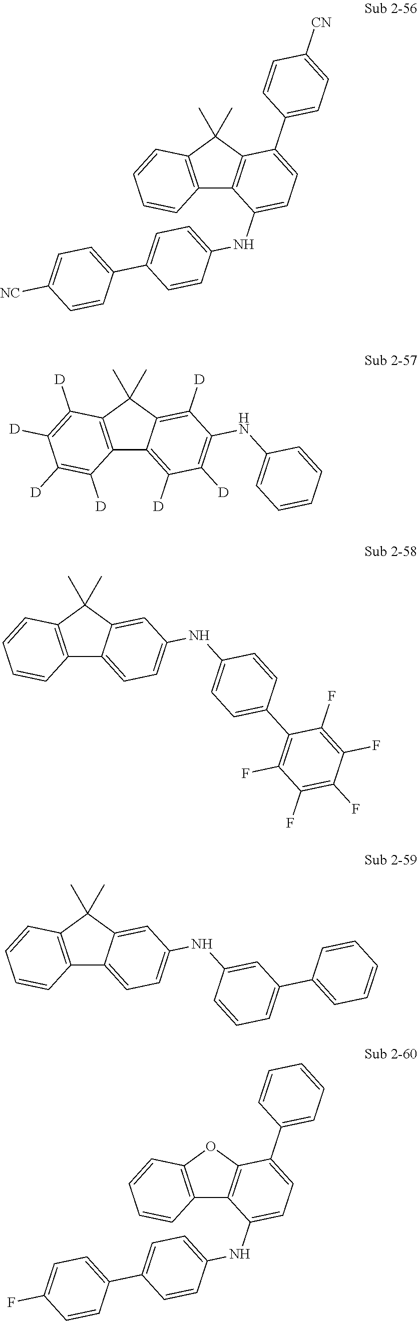 [SMILES: CC1(C)c2ccccc2-c2c(Nc3ccc(-c4ccc(C#N)cc4)cc3)ccc(-c3ccc(C#N)cc3)c21 ; [2H]c1c([2H])c([2H])c2c(c1[2H])-c1c([2H])c([2H])c(Nc3ccccc3)c([2H])c1C2(C)C ; CC1(C)c2ccccc2-c2ccc(Nc3ccc(-c4c(F)c(F)c(F)c(F)c4F)cc3)cc21 ; CC1(C)c2ccccc2-c2ccc(Nc3cccc(-c4ccccc4)c3)cc21 ; Fc1ccc(-c2ccc(Nc3ccc(-c4ccccc4)c4oc5ccccc5c34)cc2)cc1]

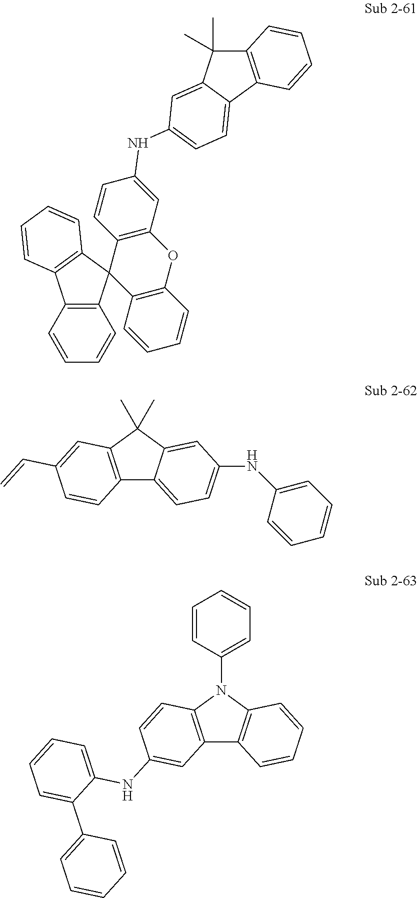

Table 2 below shows Field Desorption-Mass Spectrometry (FD-MS) values of the compounds belonging to Sub 2.

TABLE 2

| Compound | FD-MS | Compound | FD-MS |
|---|---|---|---|
| Sub 2-1 | m/z = 361.18 (C₂₇H₂₃N = 361.49) | Sub 2-2 | m/z = 285.15 (C₂₁H₁₉N = 285.39) |
| Sub 2-3 | m/z = 259.10 (C₁₈H₁₃NO = 259.31) | Sub 2-4 | m/z = 361.18 (C₂₇H₂₃N = 361.49) |
| Sub 2-5 | m/z = 351.11 (C₂₄H₁₇NS = 351 .47) | Sub 2-6 | m/z = 407.17 (C₃₁H₂₁N = 407.52) |
| Sub 2-7 | m/z = 485.21 (C₃₇H₂₇N = 485.63) | Sub 2-8 | m/z = 361.18 (C₂₇H₂₃N = 361.49) |
| Sub 2-9 | m/z = 411.20 (C₃₁H₂₅N = 411.55) | Sub 2-10 | m/z = 347.17 (C₂₆H₂₁N = 347.46) |
| Sub 2-11 | m/z = 351.13 (C₂₄H₁₇NO₂ = 351.41) | Sub 2-12 | m/z = 423.16 (C₃₁H₂₁NO = 423.52) |
| Sub 2-13 | m/z = 501.19 (C₃₆H₂₇NSi = 501.70) | Sub 2-14 | m/z = 275.08 (C₁₈H₁₃NS = 275.37) |
| Sub 2-15 | m/z = 399.05 (C₂₄H₁₇NSe = 398.37) | Sub 2-16 | m/z = 485.21 (C₃₇H₂₇N = 485.63) |
| Sub 2-17 | m/z = 377.21 (C₂₈H₂₇N = 377.53) | Sub 2-18 | m/z = 409.18 (C₃₁H₂₃N = 409.53) |
| Sub 2-19 | m/z = 411.16 (C₃₀H₂₁NO = 411.50) | Sub 2-20 | m/z = 437.21 (C₃₃H₂₇N = 437.59) |
| Sub 2-21 | m/z = 361.18 (C₂₇H₂₃N = 361.49) | Sub 2-22 | m/z = 370.24 (C₂₇H₁₄D₉N = 370.54) |
| Sub 2-23 | m/z = 345.15 (C₂₅H₁₉N = 345.45) | Sub 2-24 | m/z = 375.16 (C₂₇H₂₁NO = 375.47) |
| Sub 2-25 | m/z = 401.55 (C₃₀H₂₇N = 401.21) | Sub 2-26 | m/z = 451.19 (C₃₃H₂₅NO = 451.57) |
| Sub 2-27 | m/z = 447.20 (C₃₄H₂₅N = 447.58) | Sub 2-28 | m/z = 450.21 (C₃₃H₂₆N₂ = 450.59) |
| Sub 2-29 | m/z = 411.20 (C₃₁H₂₅N = 411.55) | Sub 2-30 | m/z = 386.18 (C₂₈H₂₂N₂ = 386.50) |
| Sub 2-31 | m/z = 425.18 (C₃₁H₂₃NO = 425.53) | Sub 2-32 | m/z = 483.20 (C₃₇H₂₅N = 483.61) |
| Sub 2-33 | m/z = 411.20 (C₃₁H₂₅N = 411.55) | Sub 2-34 | m/z = 437.14 (C₃₁H₁₉NO₂ = 437.50) |
| Sub 2-35 | m/z = 443.26 (C₃₃H₃₃N = 443.63) | Sub 2-36 | m/z = 411.20 (C₃₁H₂₅N = 411.55) |
| Sub 2-37 | m/z = 303.14 (C₂₁H₁₈FN = 303.38) | Sub 2-38 | m/z = 325.09 (C₂₂H₁₅NS = 325.43) |
| Sub 2-39 | m/z = 335.13 (C₂₄H₁₇NO = 335.41) | Sub 2-40 | m/z = 559.23 (C₄₃H₂₉N = 559.71) |
| Sub 2-41 | m/z = 334.15 (C₂₄H₁₈N₂ = 334.42) | Sub 2-42 | m/z = 335.17 (C₂₅H₂₁N = 335.45) |
| Sub 2-43 | m/z = 466.20 (C₃₃H₂₆N₂O = 466.58) | Sub 2-44 | m/z = 451.19 (C₃₃H₂₅NO = 451.57) |
| Sub 2-45 | m/z = 482.18 (C₃₃H₂₆N₂S = 482.65) | Sub 2-46 | m/z = 435.20 (C₃₃H₂₅N = 435.57) |
| Sub 2-47 | m/z = 375.16 (C₂₇H₂₁NO = 375.47) | Sub 2-48 | m/z = 410.18 (C₃₀H₂₂N₂ = 410.52) |
| Sub 2-49 | m/z = 437.21 (C₃₃H₂₇N = 437.59) | Sub 2-50 | m/z = 423.16 (C₃₁H₂₁NO = 423.52) |
| Sub 2-51 | m/z = 387.20 (C₂₉H₂₅N = 387.53) | Sub 2-52 | m/z = 385.18 (C₂₉H₂₃N = 385.51) |
| Sub 2-53 | m/z = 429.15 (C₃₀H₂₀FNO = 429.49) | Sub 2-54 | m/z = 483.20 (C₃₇H₂₅N = 483.61) |
| Sub 2-55 | m/z = 335.17 (C₂₅H₂₁N = 335.45) | Sub 2-56 | m/z = 487.20 (C₃₅H₂₅N₃ = 487.61) |
| Sub 2-57 | m/z = 292.20 (C₂₁H₁₂D₇N = 292.43) | Sub 2-58 | m/z = 451.14 (C₂₇H₁₈F₅N = 451.44) |
| Sub 2-59 | m/z = 361.18 (C₂₇H₂₃N = 361.49) | Sub 2-60 | m/z = 429.15 (C₃₀H₂₀FNO = 491.49) |
| Sub 2-61 | m/z = 539.22 (C₄₀H₂₉NO = 539.68) | Sub 2-62 | m/z = 311.17 (C₂₃H₂₁N = 311.43) |
| Sub 2-63 | m/z = 410.18 (C₃₀H₂₂N₂ = 410.52) | | |

### III. Synthesis Example of Final Compound

### P-4 Synthesis Example

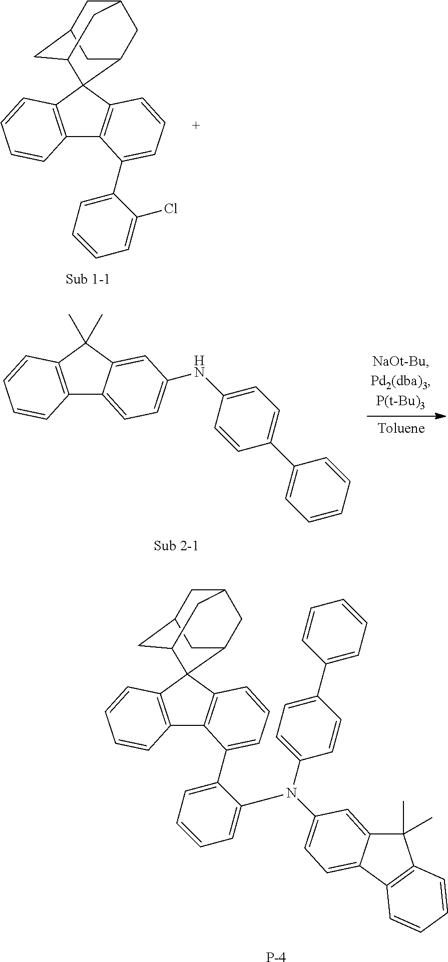

In a round bottom flask, after 16.50 g (41.57 mmol) of Sub 1-1 and 15.03 g (41.57 mmol) of Sub 2-1 were dissolved in 140 ml of toluene, 1.14 g (1.25 mmol) of Pd₂(dba)₃, 1.01 ml (2.49 mmol) of a P(t-Bu)₃ 50% toluene solution, and 11.98 g (124.70 mmol) of NaOt-Bu were added thereto, and then the mixture was stirred at 110° C. for 3 hours. After completion of the reaction, the organic layer was extracted using toluene and water, dried over MgSO₄, and the organic layer was concentrated. The produced compound was subjected to silica gel column chromatography separation and recrystallization to obtain 22.08 g (yield: 73.6%) of a product.

### P-8 Synthesis Example

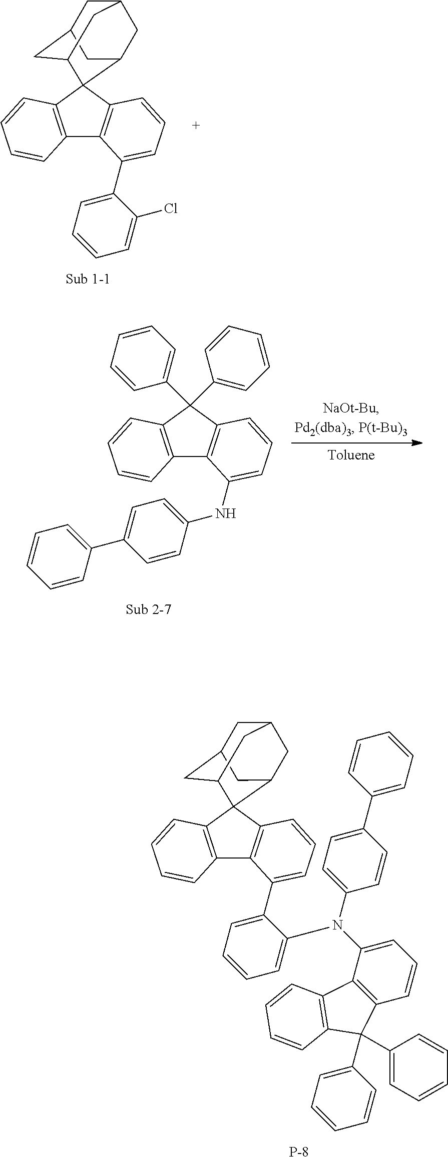

In a round bottom flask, 18.84 g (38.79 mmol) of Sub 2-7, 1.07 g (1.16 mmol) of Pd₂(dba)₃, 0.94 ml (2.33 mmol) of a P(t-Bu)₃ 50% toluene solution, 11.19 g (116.38 mmol) of NaOt-Bu, and 130 ml of toluene were added to 15.40 g (38.79 mmol) of Sub 1-1, and 27.34 g (yield: 83.3%) of a product P-8 was obtained using the above-described P-4 synthesis method.

### P-24 Synthesis Example

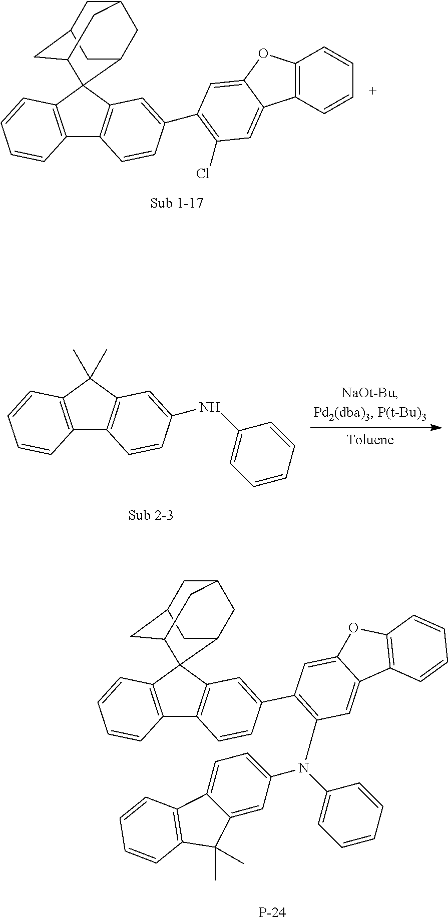

In a round bottom flask, 6.57 g (23.04 mmol) of Sub 2-3, 0.63 g (0.69 mmol) of Pd₂(dba)₃, 0.56 ml (1.38 mmol) of a P(t-Bu)₃ 50% toluene solution, 6.64 g (69.11 mmol) of NaOt-Bu, and 80 ml of toluene were added to 11.20 g (23.04 mmol) of Sub 1-17, and 10.4 g (yield: 61.5%) of a product P-24 was obtained using the above-described P-4 synthesis method.

### P-32 Synthesis Example

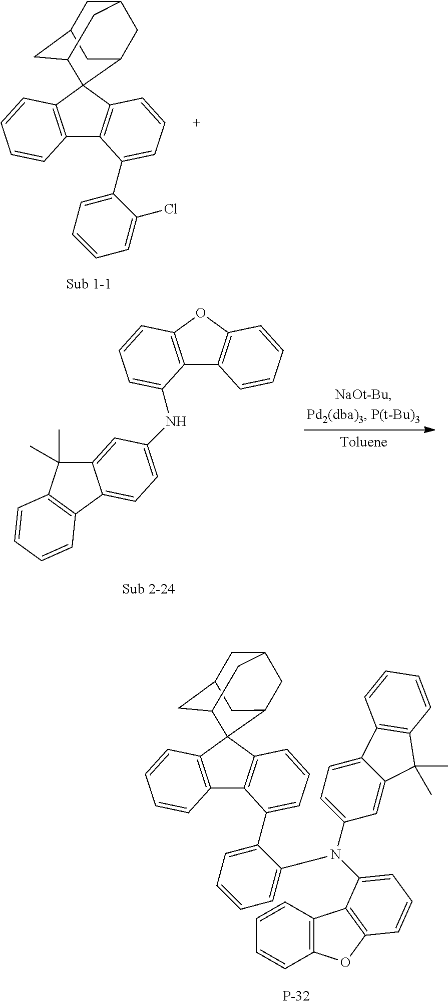

In a round bottom flask, 15.99 g (42.57 mmol) of Sub 2-24, 1.17 g (1.28 mmol) of Pd₂(dba)₃, 1.03 ml (2.55 mmol) of a P(t-Bu)₃ 50% toluene solution, 12.28 g (127.72 mmol) of NaOt-Bu, and 145 ml of toluene were added to 16.90 g (42.57 mmol) of Sub 1-1, and 22.84 g (yield: 72.9%) of a product P-36 was obtained using the above-described P-4 synthesis method.

### P-44 Synthesis Example

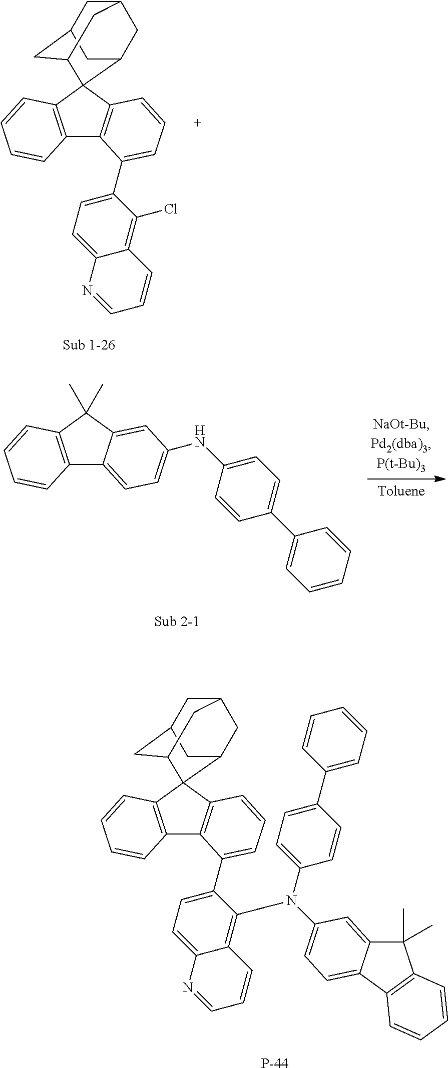

In a round bottom flask, 8.92 g (24.69 mmol) of Sub 2-1, 0.68 g (0.74 mmol) of Pd₂(dba)₃, 0.60 ml (1.48 mmol) of a P(t-Bu)₃ 50% toluene solution, 7.12 g (74.06 mmol) of NaOt-Bu, and 85 ml of toluene were added to 11.06 g (24.69 mmol) of Sub 1-26, and 11.08 g (yield: 58.1%) of a product P-44 was obtained using the above-described P-4 synthesis method.

### P-60 Synthesis Example

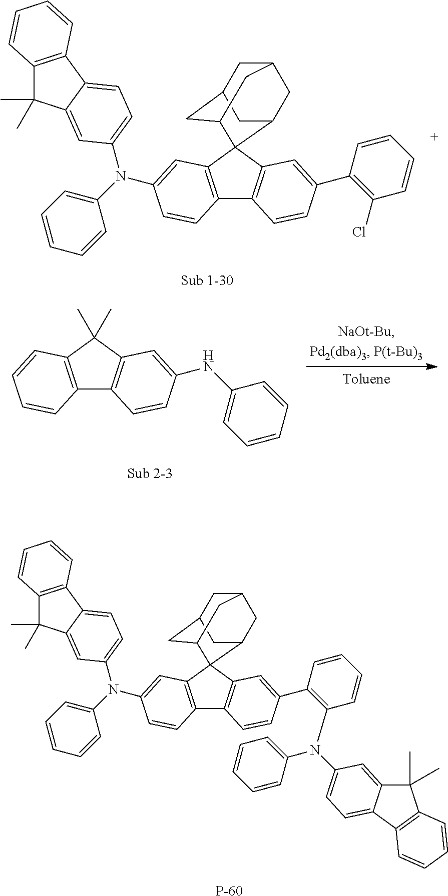

In a round bottom flask, 5.54 g (19.40 mmol) of Sub 2-3, 0.53 g (0.58 mmol) of Pd₂(dba)₃, 0.47 ml (1.16 mmol) of a P(t-Bu)₃ 50% toluene solution, 5.59 g (58.21 mmol) of NaOt-Bu, and 65 ml of toluene were added to 13.20 g (19.40 mmol) of Sub 1-30, and 12.31 g (yield: 68.3%) of a product P-60 was obtained using the above-described P-4 synthesis method.

### P-80 Synthesis Example

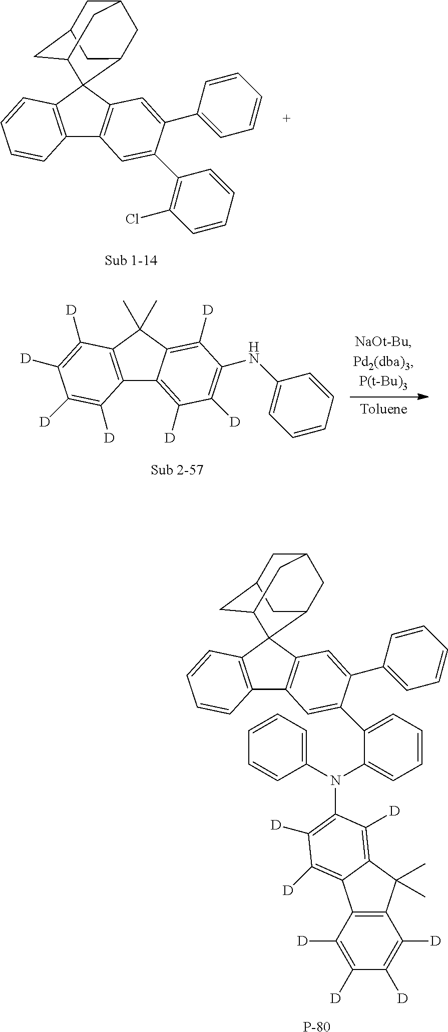

In a round bottom flask, 6.94 g (23.74 mmol) of Sub 2-57, 0.65 g (0.71 mmol) of Pd₂(dba)₃, 0.58 ml (1.42 mmol) of a P(t-Bu)₃ 50% toluene solution, 4.56 g (47.48 mmol) of NaOt-Bu, and 80 ml of toluene were added to 11.23 g (23.74 mmol) of Sub 1-14, and 12.37 g (yield: 71.5%) of a product P-80 was obtained using the above-described P-4 synthesis method.

### P-86 Synthesis Example

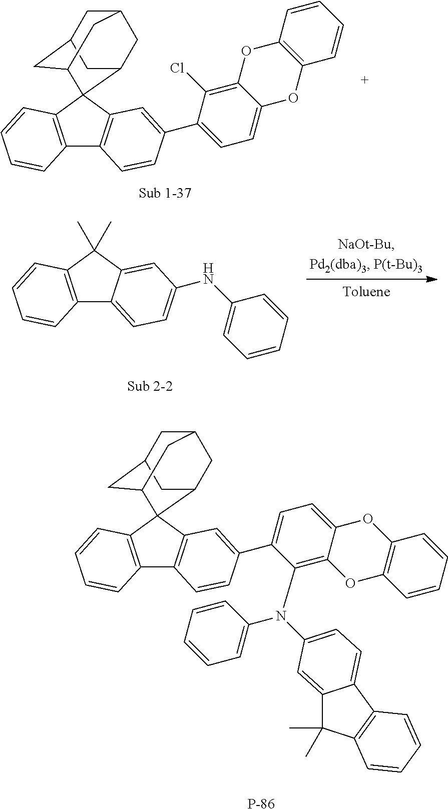

In a round bottom flask, 4.59 g (16.08 mmol) of Sub 2-2, 0.44 g (0.48 mmol) of Pd₂(dba)₃, 0.39 ml (0.96 mmol) of a P(t-Bu)₃ 50% toluene solution, 3.09 g (32.16 mmol) of NaOt-Bu, and 55 ml of toluene were added to 8.09 g (16.08 mmol) of Sub 1-37, and 8.23 g (yield: 68.1%) of a product P-86 was obtained using the above-described P-4 synthesis method.

### P-89 Synthesis Example

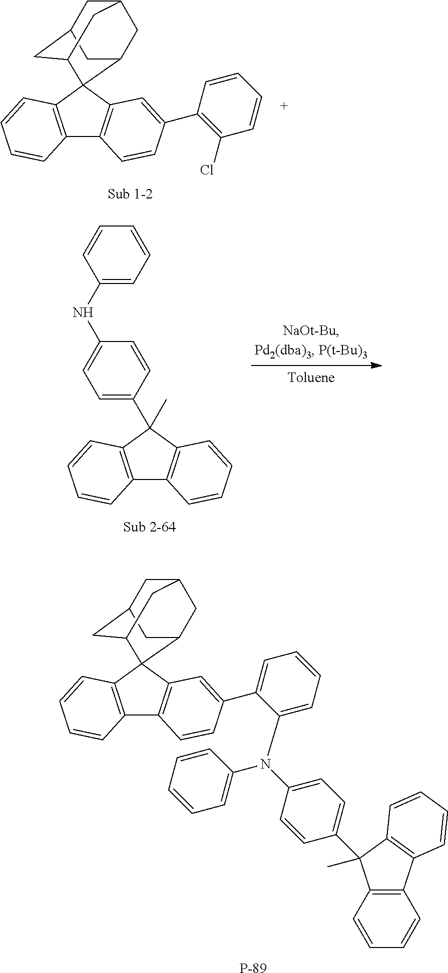

In a round bottom flask, 10.10 g (29.07 mmol) of Sub 2-64, 0.80 g (0.87 mmol) of Pd₂(dba)₃, 0.71 ml (1.74 mmol) of a P(t-Bu)₃ 50% toluene solution, 5.59 g (58.14 mmol) of NaOt-Bu, and 100 ml of toluene were added to 11.54 g (29.07 mmol) of Sub 1-2, and 11.81 g (yield: 57.4%) of a product P-89 was obtained using the above-described P-4 synthesis method.

Meanwhile, FD-MS values of Compounds P-1 to P-69 of the present disclosure prepared according to the Synthesis Examples as described above are as shown in Table 3 below.

TABLE 3

| Compound | FD-MS | Compound | FD-MS |
|---|---|---|---|
| P-1 | m/z = 721.37 (C₅₅H₄₇N = 721.99) | P-2 | m/z = 721.37 (C₅₅H₄₇N = 721.99) |
| P-3 | m/z = 751.35 (C₅₅H₄₅NO₂ = 751.97) | P-4 | m/z = 721.37 (C₅₅H₄₇N = 721.99) |
| P-5 | m/z = 711.30 (C₅₂H₄₁NS = 711.97) | P-6 | m/z = 721.37 (C₅₅H₄₇N = 721.99) |
| P-7 | m/z = 767.36 (C₅₉H₄₅N = 768.02) | P-8 | m/z = 845.40 (C₆₅H₅₁N = 846.13) |
| P-9 | m/z = 721.37 (C₅₅H₄₇N = 721.99) | P-10 | m/z = 885.43 (C₅₈H₅₅N = 886.20) |
| P-11 | m/z = 771.39 (C₅₉H₄₉N = 772.05) | P-12 | m/z = 757.37 (C₅₈H₄₇N = 758.02) |
| P-13 | m/z = 711.31 (C₅₂H₄₁NO₂ = 711.91) | P-14 | m/z = 909.40 (C₆₉H₅₁NO = 910.17) |
| P-15 | m/z = 767.32 (C₅₅H₄₅NOS = 768.03) | P-16 | m/z = 861.38 (C₆₄H₅₁NSi = 862.20) |
| P-17 | m/z = 711.30 (C₅₂H₄₁NS = 711.97) | P-18 | m/z = 759.24 (C₅₂H₄₁NSe = 758.87) |
| P-19 | m/z = 845.40 (C₆₅H₅₁N = 846.13) | P-20 | m/z = 787.42 (C₆₀H₅₃N = 788.09) |
| P-21 | m/z = 851.41 (C₆₄H₅₃NO = 852.13) | P-22 | m/z = 721.37 (C₅₅H₄₇N = 721.99) |
| P-23 | m/z = 771.35 (C₅₈H₄₅NO = 772.00) | P-24 | m/z = 735.35 (C₅₅H₄₅NO = 735.97) |
| P-25 | m/z = 797.40 (C₆₁H₅₁N = 798.09) | P-26 | m/z = 735.35 (C₅5H₄₅NO = 735.97) |
| P-27 | m/z = 721.37 (C₅₅H₄₇N = 721.99) | P-28 | m/z = 730.43 (C₅₅H₃₈D₉N = 731.04) |
| P-29 | m/z = 921.43 (C₇₁H₅₅N = 922.23) | P-30 | m/z = 747.39 (C₅₇H₄₉N = 748.03) |
| P-31 | m/z = 695.36 (C₅₃H₄₅N = 695.95) | P-32 | m/z = 735.35 (C₅₅H₄₅NO = 735.97) |
| P-33 | m/z = 761.40 (C₅₈H₅₁N = 762.05) | P-34 | m/z = 812.41 (C₆₁H₅₂N₂ = 813.10) |
| P-35 | m/z = 811.38 (C₆₁H₄₉NO = 812.07) | P-36 | m/z = 761.31 (C₅₆H₄₃NS = 762.03) |
| P-37 | m/z = 807.39 (C₆₂H₄₉N = 808.08) | P-38 | m/z = 810.40 (C₆₁H₅₀N₂ = 811.09) |
| P-39 | m/z = 902.42 (C₆₇H₅₄N₂O = 903.18) | P-40 | m/z = 771.39 (C₅₉H₄₉N = 772.05) |
| P-41 | m/z = 722.37 (C₅₄H₄₆N₂ = 722.98) | P-42 | m/z = 771.36 (C₅₇H₄₅N₃ = 772.01) |
| P-43 | m/z = 785.37 (C₅₉H47NO = 786.03) | P-44 | m/z = 772.38 (C₅₈H₄₈N₂ = 773.04) |
| P-45 | m/z = 843.39 (C₆₅H₄₉N = 844.11) | P-46 | m/z = 771.39 (C₅₉H₄₉N = 772.05) |
| P-47 | m/z = 797.33 (C₅₉H₄₃NO₂ = 798.00) | P-48 | m/z = 803.45 (C₆₁H₅₇N = 804.13) |
| P-49 | m/z = 813.40 (C₆₁H₅₁NO = 814.09) | P-50 | m/z = 771.39 (C₅₉H₄₉N = 772.05) |
| P-51 | m/z = 663.33 (C₄₉H₄₂FN = 663.88) | P-52 | m/z = 761.31 (C₅₆H₄₃NS = 762.03) |
| P-53 | m/z = 695.32 (C₅₂H₄₁NO = 695.91) | P-54 | m/z = 919.42 (C₇₁H₅₃N = 920.21) |
| P-55 | m/z = 771.39 (C₅₉H₄₉N = 772.05) | P-56 | m/z = 928.48 (C₇₀H₆₀N₂ = 929.26) |
| P-57 | m/z = 771.39 (C₅₉H₄₉N = 772.05) | P-58 | m/z = 750.40 (C₅₆H₅₀N₂ = 751.03) |
| P-59 | m/z = 695.36 (C₅₃H₄₅N = 695.95) | P-60 | m/z = 826.39 (C₆₁H₅₀N₂O = 827.08) |
| P-61 | m/z = 811.38 (C₆₁H₄₉NO = 812.07) | P-62 | m/z = 842.37 (C₆₁H₅₀N₂S = 843.15) |
| P-63 | m/z = 845.40 (C₆₅H₅₁N = 846.13) | P-64 | m/z = 812.41 (C₆₁H₅₂N₂ = 813.10) |
| P-65 | m/z = 645.34 (C₄₉H₄₃N = 645.89) | P-66 | m/z = 735.35 (C₅₅H₄₅NO = 735.97) |
| P-67 | m/z = 721.37 (C₅₅H₄₇N = 721.99) | P-68 | m/z = 770.37 (C₅₈H₄₆N₂ = 771.02) |
| P-69 | m/z = 797.40 (C₆₁H₅₁N = 798.09) | P-70 | m/z = 783.35 (C₅₉H₄₅NO = 784.02) |
| P-71 | m/z = 670.33 (C₅₀H₄₂N₂ = 670.90) | P-72 | m/z = 747.39 (C₅₇H₄₉N = 748.03) |
| P-73 | m/z = 745.37 (C₅₇H₄₇N = 746.01) | P-74 | m/z = 807.33 (C₅₈H₄₃F₂NO = 807.98) |
| P-75 | m/z = 843.39 (C₆₅H₄₉N = 844.11) | P-76 | m/z = 934.43 (C₆₈H₅₈N₂S = 935.29) |
| P-77 | m/z = 731.34 (C₅₃H₄₃F₂N = 731.93) | P-78 | m/z = 797.40 (C₆₁H₅₁N = 798.09) |
| P-79 | m/z = 847.39 (C₆₃H₄₉N₃ = 848.11) | P-80 | m/z = 728.41 (C₅₅H₄₀D₇N = 729.03) |
| P-81 | m/z = 811.32 (C₅₅H₄₂F₅N = 811.94) | P-82 | m/z = 721.37 (C₅₅H₄₇N = 721.99) |
| P-83 | m/z = 807.33 (C₅₈H₄₃F₂NO = 807.98) | P-84 | m/z = 807.44 (C₆₀H₅₇NO = 808.12) |
| P-85 | m/z = 899.41 (C₆₈H₅₃NO = 900.18) | P-86 | m/z = 751.35 (C₅₅H₄₅NO₂ = 751.97) |
| P-87 | m/z = 747.39 (C₅₇H₄₉N = 748.03) | P-88 | m/z = 770.37 (C₅₈H₄₆N₂ = 771.02) |
| P-89 | m/z = 707.36 (C₅₄H₄₅N = 707.96) | P-90 | m/z = 681.34 (C₅₂H₄₃N = 681.92) |

Manufacturing Evaluation of Organic Electric Devices

### (Example 1) Green Organic Light Emitting Device (Hole Transport Layer)

An organic electroluminescent device was manufactured according to a conventional method using the compound of the present disclosure as a hole transport layer material.

First, a hole injection layer was formed by vacuum-depositing N¹-(naphthalen-2-yl)-N⁴,N⁴-bis(4-(naphthalen-2-yl(phenyl)amino)phenyl)-N1-phenylbenzene-1,4-diamine (hereinafter, abbreviated as 2-TNATA) to a thickness of 60 nm on an ITO layer (anode) formed on a glass substrate.

A hole transport layer was formed by vacuum-depositing the Compound P-2 of the present disclosure as a hole transport compound to a thickness of 60 nm on the hole injection layer.

A light emitting layer with a thickness of 30 nm was deposited by doping 4,4′-N,N′-dicarbazole-biphenyl (hereinafter, abbreviated as CBP) as a host and tris(2-phenylpyridine)-iridium (hereinafter, abbreviated as Ir(ppy)₃) as a dopant at a weight ratio of 90:10 on the upper portion of the hole transport layer.

A hole blocking layer was formed by vacuum-depositing (1,1′-biphenyl-4-olato)bis(2-methyl-8-quinolinolato)aluminum (hereinafter abbreviated as BAlq) to a thickness of 5 nm on the light emitting layer.

An electron transport layer was formed by vacuum-depositing Tris(8-quinolinol)aluminum (hereinafter abbreviated as Alq₃) to a thickness of 40 nm on the hole blocking layer.

Thereafter, an organic electroluminescent device was manufactured by depositing an alkali metal halide LiF to a thickness of 0.2 nm to form an electron injection layer, and subsequently depositing Al to a thickness of 150 nm to form a cathode.

### (Example 2) to (Example 14)

An organic electroluminescent device was manufactured in the same manner as in Example 1 except that the compounds of the present disclosure described in Table 4 below were used instead of the Compound P-2 of the present disclosure as the hole transport layer material.

### Comparative Example 1

An organic electroluminescent device was manufactured in the same manner as in Example 1 above except that N,N′-Bis(1-naphthalenyl)-N,N′-bis-phenyl-(1,1′-biphenyl)-4,4′-diamine (hereinafter abbreviated as NPB) was used as the hole transport layer material.

### (Comparative Example 2) to (Comparative Example 4)

An organic electroluminescent device was manufactured in the same manner as in Example 1 above except that the Comparative Compounds 1 to 3 below were used as the hole transport layer material.

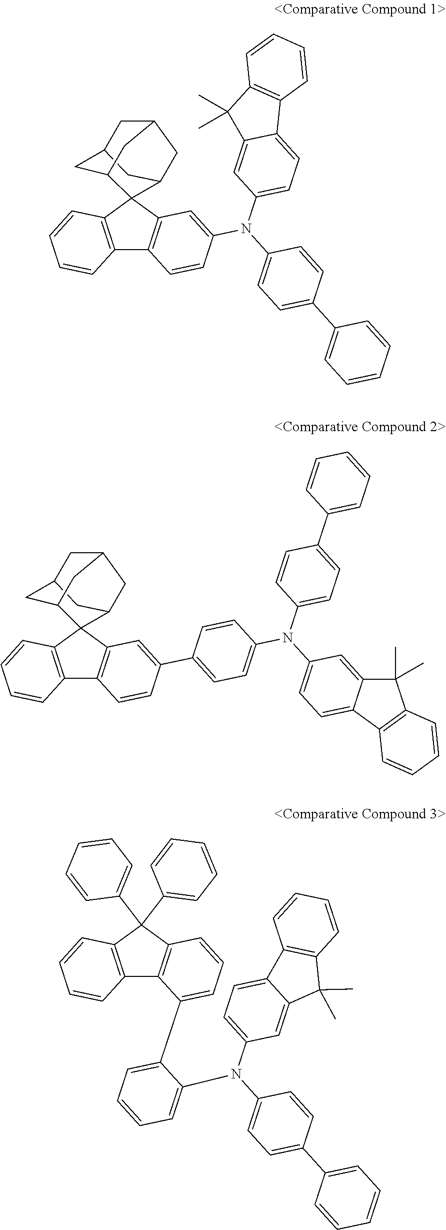 [SMILES: CC1(C)c2ccccc2-c2ccc(N(c3ccc(-c4ccccc4)cc3)c3ccc4c(c3)C3(c5ccccc5-4)C4CC5CC(C4)CC3C5)cc21 ; CC1(C)c2ccccc2-c2ccc(N(c3ccc(-c4ccccc4)cc3)c3ccc(-c4ccc5c(c4)C4(c6ccccc6-5)C5CC6CC(C5)CC4C6)cc3)cc21 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

A forward bias DC voltage was applied to the organic electroluminescent devices manufactured according to Examples 1 to 20 and Comparative Examples 1 to 4 above, and electroluminescence (EL) characteristics were measured with PR-650 manufactured by Photo Research Inc., and the T95 lifespans were measured using a lifespan measuring device manufactured by McScience Inc. at a reference luminance of 5,000 cd/m². Table 4 below shows the manufactured devices and evaluation results.

TABLE 4

| | | Driving | Current | | | | | |
|---|---|---|---|---|---|---|---|---|
| | | voltage | density | Luminance | Efficiency | T | CIE | |
| | Compound | (V) | (mA/cm²) | (cd/m²) | (cd/A) | (95) | X | Y |
| Comparative | NPB | 6.0 | 21.4 | 5000 | 23.4 | 58.6 | 0.31 | 0.61 |
| Example (1) | | | | | | | | |
| Comparative | Comparative | 5.8 | 19.2 | 5000 | 26.0 | 72.4 | 0.32 | 0.60 |
| Example (2) | Compound 1 | | | | | | | |
| Comparative | Comparative | 5.6 | 18.2 | 5000 | 27.5 | 83.2 | 0.32 | 0.60 |
| Example (3) | Compound 2 | | | | | | | |
| Comparative | Comparative | 5.6 | 17.4 | 5000 | 28.8 | 79.7 | 0.32 | 0.60 |
| Example (4) | Compound 3 | | | | | | | |
| Example (1) | P-2 | 5.1 | 12.8 | 5000 | 39.2 | 107.3 | 0.32 | 0.60 |
| Example (2) | P-4 | 5.1 | 12.5 | 5000 | 40.0 | 108.9 | 0.32 | 0.60 |
| Example (3) | P-12 | 5.2 | 13.1 | 5000 | 38.3 | 106.2 | 0.33 | 0.60 |
| Example (4) | P-21 | 5.2 | 13.6 | 5000 | 36.8 | 103.4 | 0.33 | 0.61 |
| Example (5) | P-24 | 5.2 | 13.4 | 5000 | 37.4 | 104.7 | 0.33 | 0.60 |
| Example (6) | P-31 | 5.2 | 12.9 | 5000 | 38.9 | 106.8 | 0.33 | 0.61 |
| Example (7) | P-41 | 5.4 | 14.4 | 5000 | 34.8 | 100.0 | 0.33 | 0.60 |
| Example (8) | P-42 | 5.4 | 14.7 | 5000 | 33.9 | 98.7 | 0.33 | 0.61 |
| Example (9) | P-61 | 5.3 | 14.0 | 5000 | 35.7 | 101.8 | 0.33 | 0.63 |
| Example (10) | P-69 | 5.2 | 13.2 | 5000 | 37.9 | 105.5 | 0.33 | 0.63 |
| Example (11) | P-83 | 5.4 | 15.5 | 5000 | 32.2 | 96.9 | 0.33 | 0.63 |
| Example (12) | P-85 | 5.4 | 15.3 | 5000 | 32.7 | 97.2 | 0.33 | 0.63 |
| Example (13) | P-87 | 5.4 | 14.3 | 5000 | 35.0 | 99.6 | 0.33 | 0.62 |
| Example (14) | P-89 | 5.3 | 13.9 | 5000 | 36.1 | 103.0 | 0.33 | 0.62 |

It can be confirmed from Table 4 above that the devices using the compounds represented by Chemical Formula 1 of the present disclosure as the hole transport layer material have remarkably improved electrical properties compared to the devices using the Comparative Compounds as the hole transport layer material.

That is, the devices of Comparative Examples 2 to 4 manufactured using Comparative Compounds 1 to 3 containing a fluorenyl group in an amine group rather than the device of Comparative Example 1 manufactured using NPB mainly used as a hole transport layer material had improved electrical properties (driving voltage, efficiency, and lifespan). In the devices using the compounds according to Chemical Formula 1 of the present disclosure as the hole transport layer material compared to the devices of Comparative Examples 2 to 4, the electrical properties of the devices were improved as the driving voltages, the luminous efficiencies and lifespans of the organic electroluminescent devices were improved.

First, Comparative Compounds 1 to 3 and the compounds represented by Chemical Formula 1 of the present disclosure have a similar basic composition containing an amine group and a fluorenyl group in the structure in a broad framework. However, based on the compound of the present disclosure, Comparative Compound 1 is different from the compounds of the present disclosure in that the amine group is directly bonded to spiro-adamantane fluorene

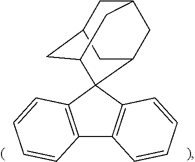 [SMILES: c1ccc2c(c1)-c1ccccc1C21C2CC3CC(C2)CC1C3]

and Comparative Compound 2 is different from the compounds of the present disclosure in that a para-phenyl linking group is introduced between spiro-adamantane fluorene and the amine group, and Comparative Compound 3 is different from the compounds of the present disclosure in that an ortho-phenyl linking group is introduced between diphenylfluorene and the amine group, but a spiro-adamantane fluorene skeleton is not included in the structure.

First, looking at Comparative Example 2 and Comparative Examples 3 to 4, it can be confirmed that Comparative Examples 3 and 4 exhibit improved device properties compared to Comparative Example 2 by having a linking group between the amine group and the fluorenyl group (spiro-adamantane fluorene/diphenyl fluorene). It can be seen from this that whether or not a linking group is introduced between the amine group and the fluorenyl group affects the electrical properties of the devices.

In addition, looking at Comparative Examples 3 and 4, it can be confirmed that all of them are similar in that a linking group is introduced between the amine group and the fluorenyl group, but Comparative Example 3 including a spiro-adamantane fluorene skeleton has improved device properties in terms of lifespan, and Comparative Example 4 including an ortho-phenyl linking group has improved device properties in terms of efficiency, and it can be seen from this that the degree of bending of the linking group or the configuration shape of the fluorenyl group affects the electrical properties of the devices.

Through the results of these Comparative Examples, it can be confirmed that the compounds of the present disclosure represented by Chemical Formula 1 of the present disclosure include an amine group and a spiro-adamantane fluorene skeleton, and a linking group bent by ortho is necessarily present between them so that the device properties of Examples 1 to 14 are remarkably improved compared to Comparative Examples 2 to 4.

Through the device results of the compounds of the present disclosure and the Comparative Compounds, the energy levels (HOMO, LUMO, T1, etc.) of the compounds may significantly vary depending on whether or not the linking group is between the amine group and the fluorenyl group, the substitution form of the linking group, the configuration shape of the fluorenyl group, etc., and since these differences in compound properties act as a major factor in improving device performance during device deposition, it is suggested that different device results as described above may be derived.

Furthermore, it can be confirmed that having a structure in which the fluorenyl group is spiro-adamantane fluorene and an ortho-linking group is connected between the fluorenyl group and the amine group as shown in the chemical formula of the present disclosure is a structure suitable for improving the performance of the devices.

In addition, the properties of devices in which the compounds of the present disclosure are applied to the hole transport layer have been described in the above-described device manufacturing evaluation results, but the compounds of the present disclosure may be applied to one or more of the light emitting layer, the hole transport layer, and the light emitting auxiliary layer.

The above description is merely for exemplarily explaining the present disclosure, and those skilled in the art to which the present disclosure pertains will be able to make various modifications, such as a method for improving performance by including other compounds within a range that does not depart from the essential characteristics of the present disclosure.

Accordingly, the embodiments disclosed in the present specification are not intended to limit the present disclosure, but to explain the present disclosure, and the scope of the spirit of the present disclosure is not limited by these embodiments. The protection scope of the present disclosure should be construed by the claims below, and all technologies within the scope equivalent thereto should be construed as being included in the right scope of the present disclosure.

### EXPLANATION OF REFERENCE NUMERALS

100, 200, 300: Organic electric device 110: First electrode
120: Hole injection layer 130: Hole transport layer
140: Light emitting layer 150: Electron transport layer
160: Electron injection layer 170: Second electrode
180: Capping layer 210: Buffer layer
220: Light emitting auxiliary layer 320: First hole injection layer
330: First hole transport layer 340: First light emitting layer
350: First electron transport layer 360: First charge generation layer
361: Second charge generation layer 420: Second hole injection layer
430: Second hole transport layer 440: Second light emitting layer
450: Second electron transport layer CGL: Charge generation layer
ST1: First stack ST2: Second stack

1. A compound represented by the following Chemical Formula 1: 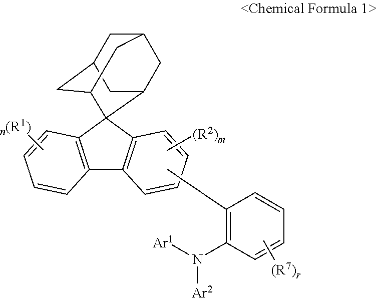
in Chemical Formula 1,
1) Ar¹ to Ar² are each independently a C₆-C₆₀ aryl group; a fluorenyl group; a C₂-C₆₀ heterocyclic group containing at least one heteroatom of O, N, S, Si, and P; or a fused ring group of a C₃-C₆₀ aliphatic ring and a C₆-C₆₀ aromatic ring; a C₁-C₃₀ alkyl group; a C₂-C₃₀ alkenyl group; a C₂-C₃₀ alkynyl group; a C₁-C₃₀ alkoxyl group; a C₆-C₃₀ aryloxy group; -Lₐ-N(Rₐ)(R_{b}); or combinations thereof, or
2) at least one of Ar¹ and Ar² may be represented by Chemical Formula 1-a or Chemical Formula 1-b below, 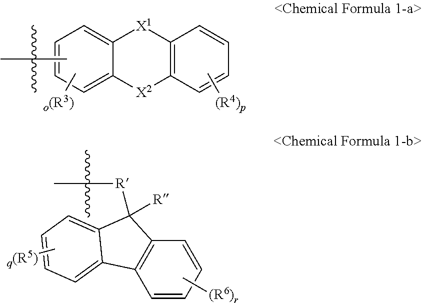
3) R¹ to R⁶, R′, and R″ are each independently hydrogen; deuterium; halogen; a cyano group; a nitro group; a C₆-C₆₀ aryl group; a fluorenyl group; a C₂-C₆₀ heterocyclic group containing at least one heteroatom of O, N, S, Si, and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; or -Lₐ-N(Rₐ)(R_{b}); or adjacent groups may be bonded to each other to form a ring,
4) Rₐ to R_{b} are each independently a C₆-C₆₀ aryl group; a fluorenyl group; a C₂-C₆₀ heterocyclic group containing at least one heteroatom of O, N, S, Si, and P; or a fused ring group of a C₃-C₆₀ aliphatic ring and a C₆-C₆₀ aromatic ring; or combinations thereof,
5) Lₐ is a single bond; a C₆-C₆₀ arylene group; a fluorenylene group; a C₂-C₆₀ heterocyclic group containing at least one heteroatom of O, N, S, Si, and P; a fused ring group of a C₃-C₆₀ aliphatic ring and a C₆-C₆₀ aromatic ring; or combinations thereof,
6) R⁷ is hydrogen; deuterium; halogen; a cyano group; a nitro group; a C₆-C₆₀ aryl group; a fluorenyl group; a C₂-C₆₀ heterocyclic group containing at least one heteroatom of O, N, S, Si, and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; or a C₆-C₃₀ aryloxy group; or adjacent groups may be bonded to each other to form a ring,
7) X¹ is CR′R″, NR′, O, S, Se, or SiR′R″,
8) X² is CR′R″, NR′, O, S, Se, SiR′R″, or a single bond,
9) n, p, q, and r are each independently an integer of 0 to 4,
10) m and o are each independently an integer of 0 to 3,
11) Ar¹ to Ar², R¹ to R⁷, R′, R″, Rₐ to R_{b}, Lₐ, and the rings formed by bonding neighboring groups to each other may each be further substituted with one or more substituents selected from the group consisting of deuterium; halogen; a silane group substituted or unsubstituted with a C₁-C₂₀ alkyl group or a C₆-C₂₀ aryl group; a siloxane group; a boron group; a germanium group; a cyano group; an amino group; a nitro group; a C₁-C₂₀ alkylthio group; a C₁-C₂₀ alkoxy group; a C₆-C₂₀ arylalkoxy group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group containing at least one heteroatom selected from the group consisting of O, N, S, Si, and P; a C₃-C₂₀ aliphatic ring group; a C₇-C₂₀ arylalkyl group; a C₈-C₂₀ arylalkenyl group; and combinations thereof, and adjacent substituents may form a ring with each other.

2. The compound of claim 1, wherein Chemical Formula 1 above is represented by any one of the following Chemical Formulas 2 to 4: 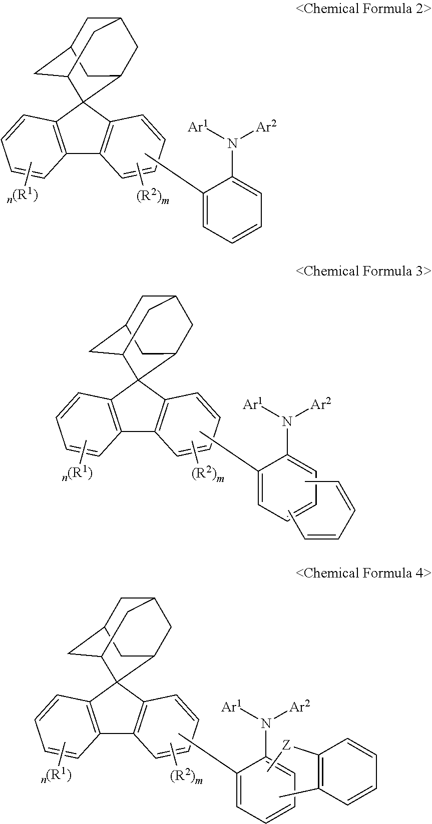
in Chemical Formulas 2 to 4,
1) Z is CR′R″, NR′, O, S, Se, SiR′R″, or a single bond, and
2) Ar¹ to Ar², R′, R″, and R¹ to R² are the same as defined in Chemical Formula 1 of claim 1.

3. The compound of claim 1, wherein Chemical Formula 1 above is represented by the following Chemical Formula 5 or Chemical Formula 6: 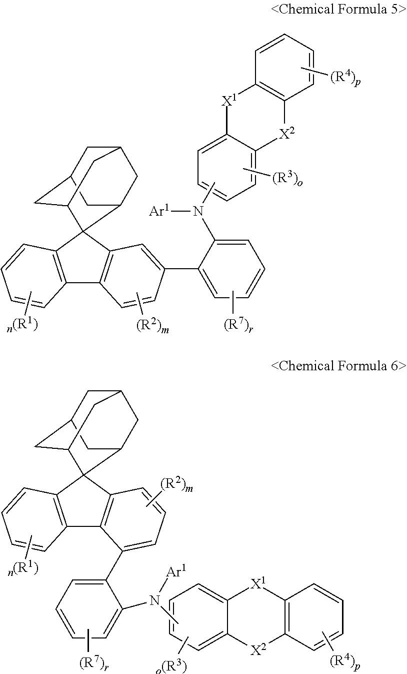
in Chemical Formula 5 and Chemical Formula 6,
X¹, X², Ar¹, R¹ to R⁴, R⁷, m to p, and r are the same as defined in Chemical Formula 1 of claim 1.

4. The compound of claim 1, wherein Chemical Formula 1 above is represented by the following Chemical Formula 7 or Chemical Formula 8: 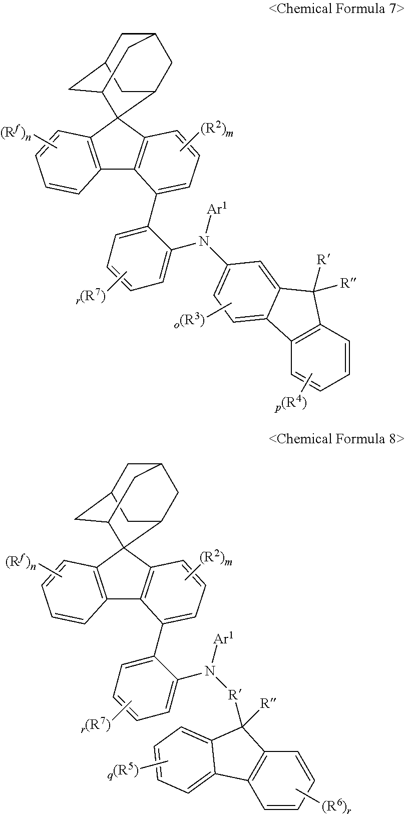
in Chemical Formulas 7 and 8,
Ar¹, R¹ to R⁷, R′, R″, and m to r are the same as defined in Chemical Formula 1 of claim 1.

5. The compound of claim 1, wherein Chemical Formula 1-a is represented by any one of the following Chemical Formulas 1-a-1 to 1-a-5: 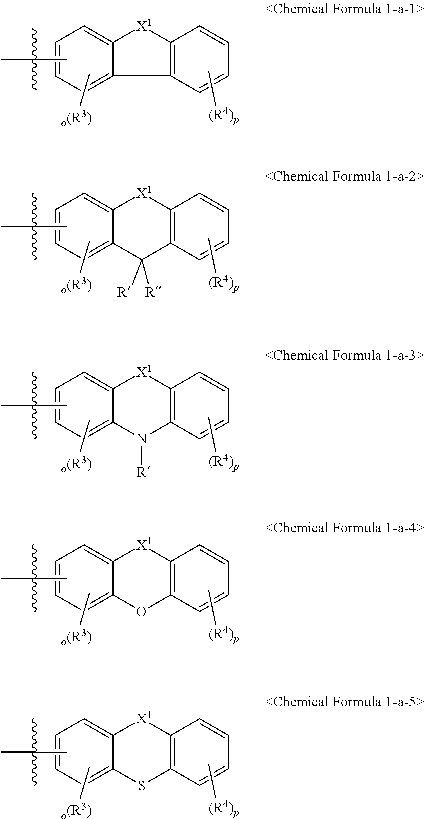
in Chemical Formula 1-a-1 to Chemical Formula 1-a-5,
X¹, R³ to R⁴, R′, R″, o, and p are the same as defined in Chemical Formula 1 of claim 1.

6. The compound of claim 1, wherein the compound represented by Chemical Formula 1 above is one of the following P-1 to P-90:

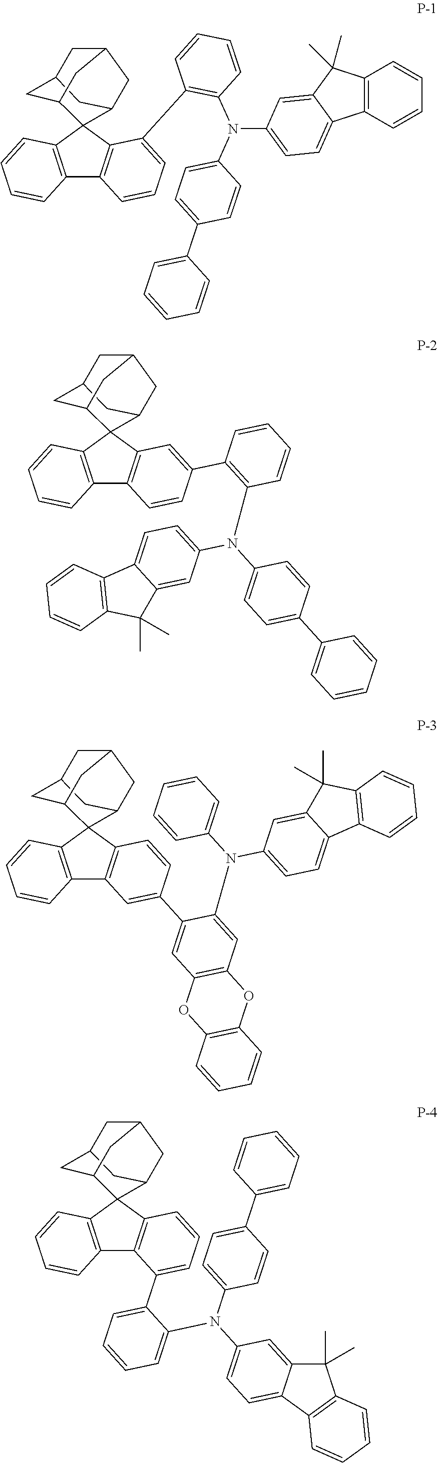 [SMILES: CC1(C)c2ccccc2-c2ccc(N(c3ccc(-c4ccccc4)cc3)c3ccccc3-c3cccc4c3C3(c5ccccc5-4)C4CC5CC(C4)CC3C5)cc21 ; CC1(C)c2ccccc2-c2ccc(N(c3ccc(-c4ccccc4)cc3)c3ccccc3-c3ccc4c(c3)C3(c5ccccc5-4)C4CC5CC(C4)CC3C5)cc21 ; CC1(C)c2ccccc2-c2ccc(N(c3ccccc3)c3cc4c(cc3-c3ccc5c(c3)-c3ccccc3C53C5CC6CC(C5)CC3C6)Oc3ccccc3O4)cc21 ; CC1(C)c2ccccc2-c2ccc(N(c3ccc(-c4ccccc4)cc3)c3ccccc3-c3cccc4c3-c3ccccc3C43C4CC5CC(C4)CC3C5)cc21]

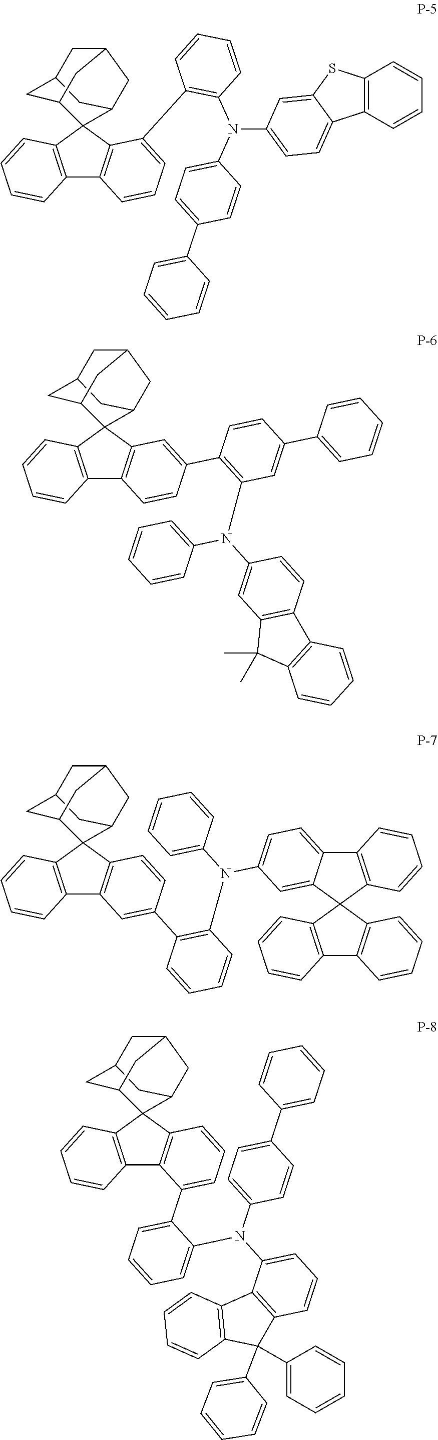 [SMILES: c1ccc(-c2ccc(N(c3ccc4c(c3)sc3ccccc34)c3ccccc3-c3cccc4c3C3(c5ccccc5-4)C4CC5CC(C4)CC3C5)cc2)cc1 ; CC1(C)c2ccccc2-c2ccc(N(c3ccccc3)c3cc(-c4ccccc4)ccc3-c3ccc4c(c3)C3(c5ccccc5-4)C4CC5CC(C4)CC3C5)cc21 ; c1ccc(N(c2ccc3c(c2)C2(c4ccccc4-c4ccccc42)c2ccccc2-3)c2ccccc2-c2ccc3c(c2)-c2ccccc2C32C3CC4CC(C3)CC2C4)cc1 ; c1ccc(-c2ccc(N(c3ccccc3-c3cccc4c3-c3ccccc3C43C4CC5CC(C4)CC3C5)c3cccc4c3-c3ccccc3C4(c3ccccc3)c3ccccc3)cc2)cc1]

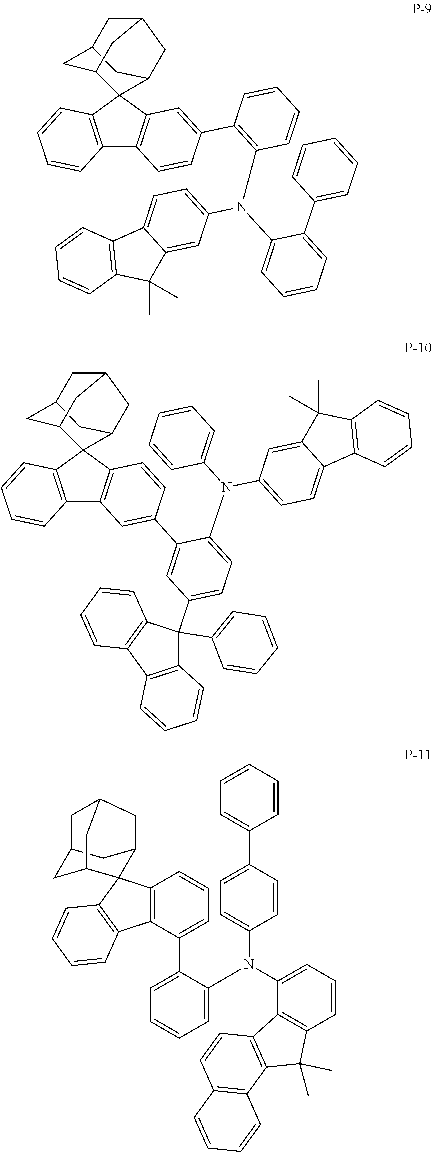 [SMILES: CC1(C)c2ccccc2-c2ccc(N(c3ccccc3-c3ccccc3)c3ccccc3-c3ccc4c(c3)C3(c5ccccc5-4)C4CC5CC(C4)CC3C5)cc21 ; CC1(C)c2ccccc2-c2ccc(N(c3ccccc3)c3ccc(C4(c5ccccc5)c5ccccc5-c5ccccc54)cc3-c3ccc4c(c3)-c3ccccc3C43C4CC5CC(C4)CC3C5)cc21 ; CC1(C)c2cccc(N(c3ccc(-c4ccccc4)cc3)c3ccccc3-c3cccc4c3-c3ccccc3C43C4CC5CC(C4)CC3C5)c2-c2ccc3ccccc3c21]

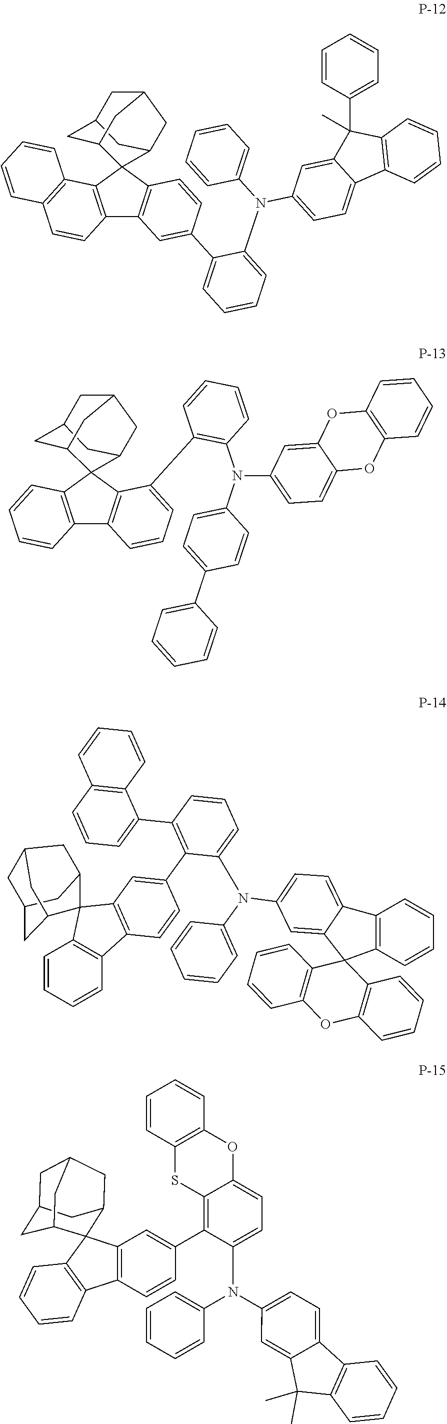 [SMILES: CC1(c2ccccc2)c2ccccc2-c2ccc(N(c3ccccc3)c3ccccc3-c3ccc4c(c3)-c3ccc5ccccc5c3C43C4CC5CC(C4)CC3C5)cc21 ; c1ccc(-c2ccc(N(c3ccc4c(c3)Oc3ccccc3O4)c3ccccc3-c3cccc4c3C3(c5ccccc5-4)C4CC5CC(C4)CC3C5)cc2)cc1 ; c1ccc(N(c2ccc3c(c2)C2(c4ccccc4Oc4ccccc42)c2ccccc2-3)c2cccc(-c3cccc4ccccc34)c2-c2ccc3c(c2)C2(c4ccccc4-3)C3CC4CC(C3)CC2C4)cc1 ; CC1(C)c2ccccc2-c2ccc(N(c3ccccc3)c3ccc4c(c3-c3ccc5c(c3)C3(c6ccccc6-5)C5CC6CC(C5)CC3C6)Sc3ccccc3O4)cc21]

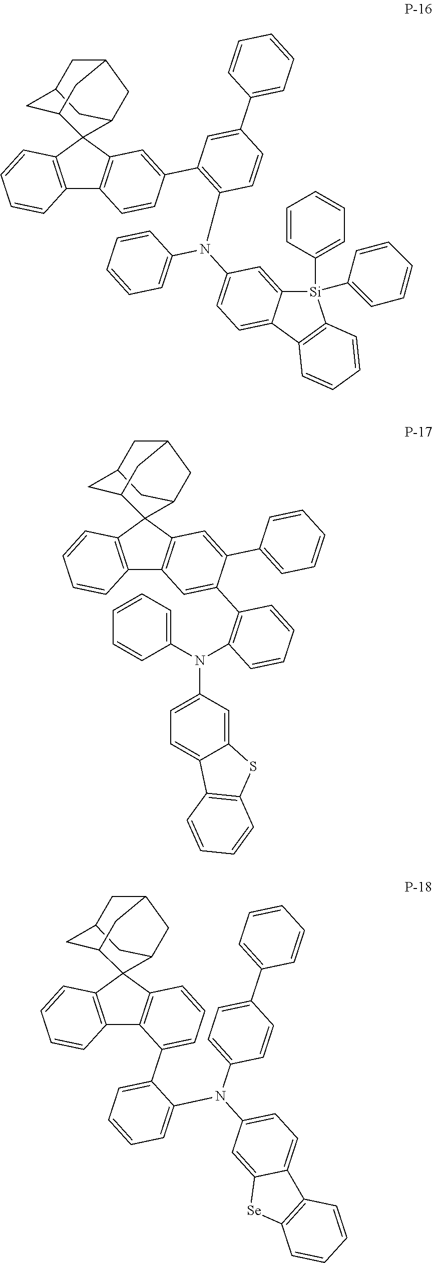 [SMILES: c1ccc(-c2ccc(N(c3ccccc3)c3ccc4c(c3)[Si](c3ccccc3)(c3ccccc3)c3ccccc3-4)c(-c3ccc4c(c3)C3(c5ccccc5-4)C4CC5CC(C4)CC3C5)c2)cc1 ; c1ccc(-c2cc3c(cc2-c2ccccc2N(c2ccccc2)c2ccc4c(c2)sc2ccccc24)-c2ccccc2C32C3CC4CC(C3)CC2C4)cc1 ; c1ccc(-c2ccc(N(c3ccc4c(c3)[se]c3ccccc34)c3ccccc3-c3cccc4c3-c3ccccc3C43C4CC5CC(C4)CC3C5)cc2)cc1]

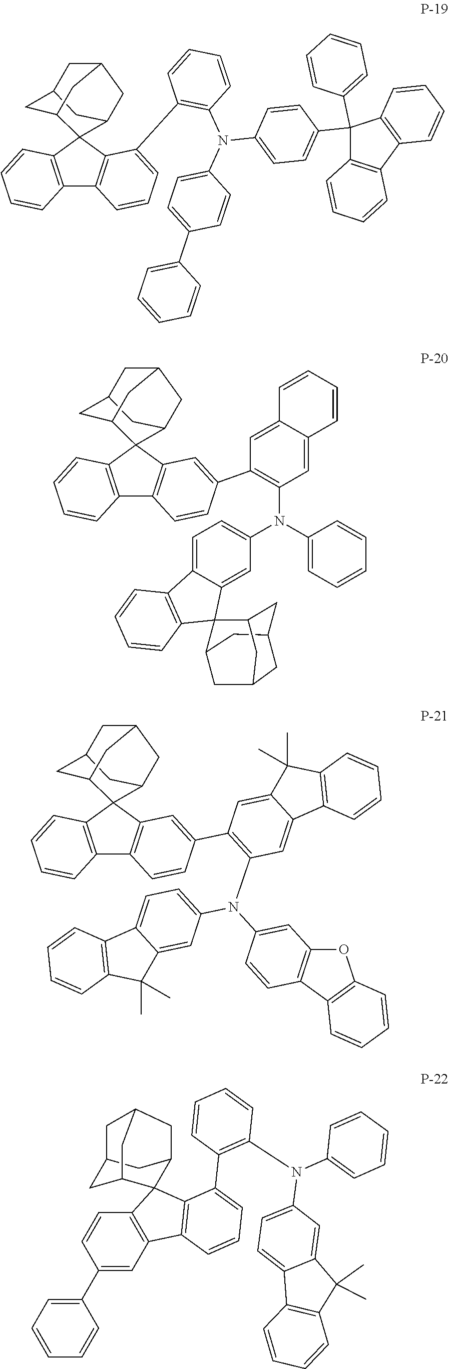

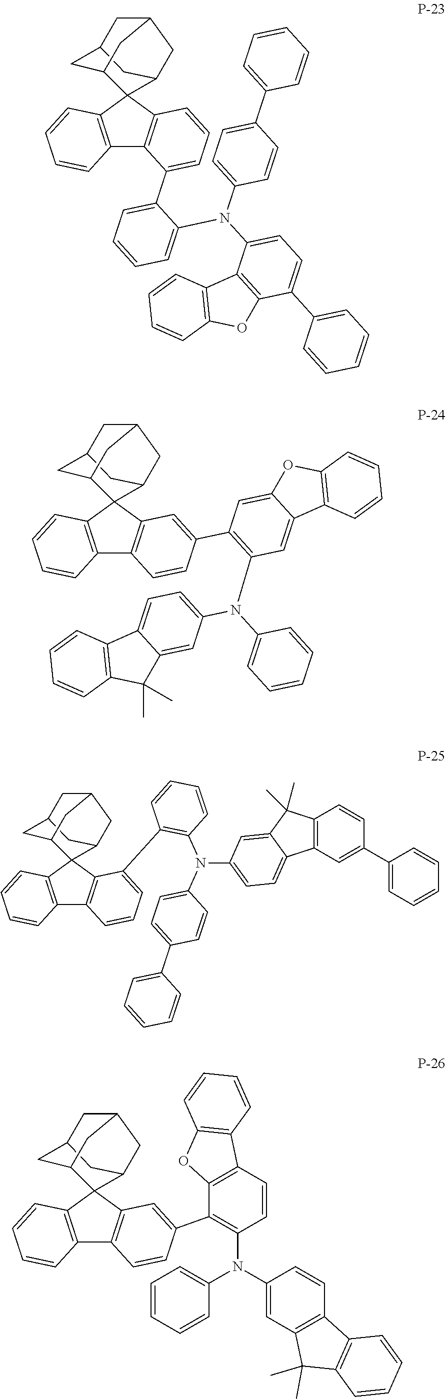 [SMILES: c1ccc(-c2ccc(N(c3ccccc3-c3cccc4c3-c3ccccc3C43C4CC5CC(C4)CC3C5)c3ccc(-c4ccccc4)c4oc5ccccc5c34)cc2)cc1 ; CC1(C)c2ccccc2-c2ccc(N(c3ccccc3)c3cc4c(cc3-c3ccc5c(c3)C3(c6ccccc6-5)C5CC6CC(C5)CC3C6)oc3ccccc34)cc21 ; CC1(C)c2ccc(-c3ccccc3)cc2-c2ccc(N(c3ccc(-c4ccccc4)cc3)c3ccccc3-c3cccc4c3C3(c5ccccc5-4)C4CC5CC(C4)CC3C5)cc21 ; CC1(C)c2ccccc2-c2ccc(N(c3ccccc3)c3ccc4c(oc5ccccc54)c3-c3ccc4c(c3)C3(c5ccccc5-4)C4CC5CC(C4)CC3C5)cc21]

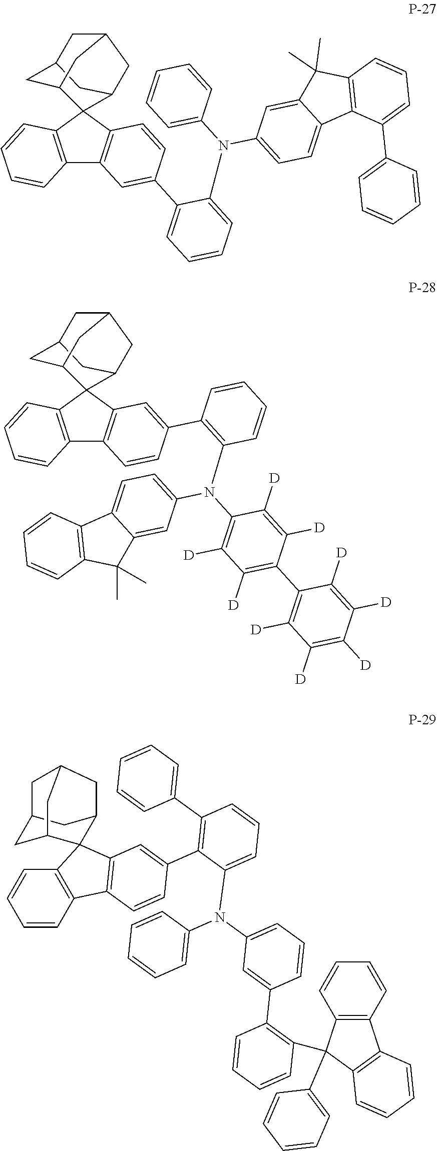 [SMILES: CC1(C)c2cc(N(c3ccccc3)c3ccccc3-c3ccc4c(c3)-c3ccccc3C43C4CC5CC(C4)CC3C5)ccc2-c2c(-c3ccccc3)cccc21 ; [2H]c1c([2H])c([2H])c(-c2c([2H])c([2H])c(N(c3ccc4c(c3)C(C)(C)c3ccccc3-4)c3ccccc3-c3ccc4c(c3)C3(c5ccccc5-4)C4CC5CC(C4)CC3C5)c([2H])c2[2H])c([2H])c1[2H] ; c1ccc(-c2cccc(N(c3ccccc3)c3cccc(-c4ccccc4C4(c5ccccc5)c5ccccc5-c5ccccc54)c3)c2-c2ccc3c(c2)C2(c4ccccc4-3)C3CC4CC(C3)CC2C4)cc1]

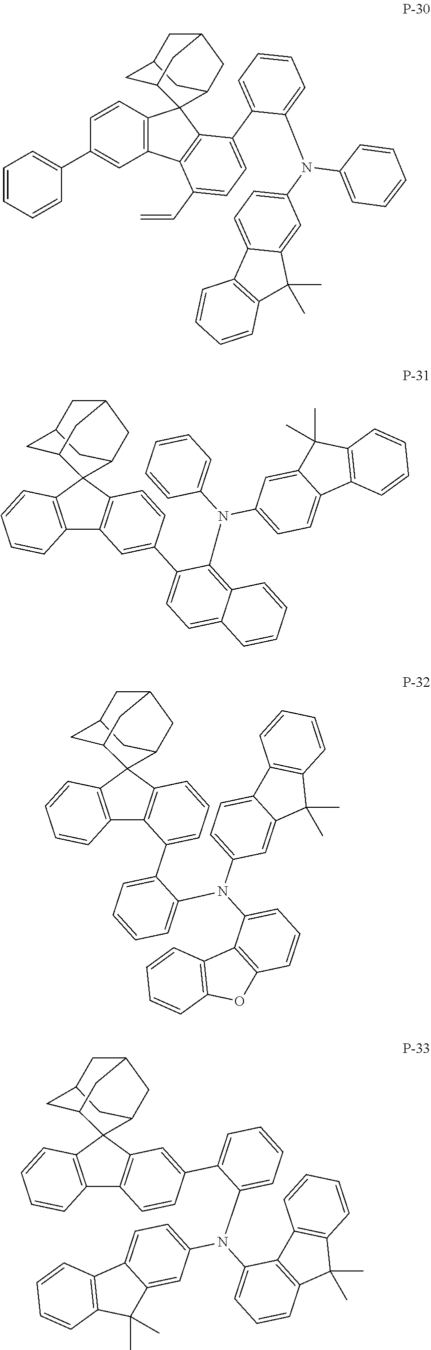 [SMILES: C=Cc1ccc(-c2ccccc2N(c2ccccc2)c2ccc3c(c2)C(C)(C)c2ccccc2-3)c2c1-c1cc(-c3ccccc3)ccc1C21C2CC3CC(C2)CC1C3 ; CC1(C)c2ccccc2-c2ccc(N(c3ccccc3)c3c(-c4ccc5c(c4)-c4ccccc4C54C5CC6CC(C5)CC4C6)ccc4ccccc34)cc21 ; CC1(C)c2ccccc2-c2ccc(N(c3ccccc3-c3cccc4c3-c3ccccc3C43C4CC5CC(C4)CC3C5)c3cccc4oc5ccccc5c34)cc21 ; CC1(C)c2ccccc2-c2ccc(N(c3ccccc3-c3ccc4c(c3)C3(c5ccccc5-4)C4CC5CC(C4)CC3C5)c3cccc4c3-c3ccccc3C4(C)C)cc21]

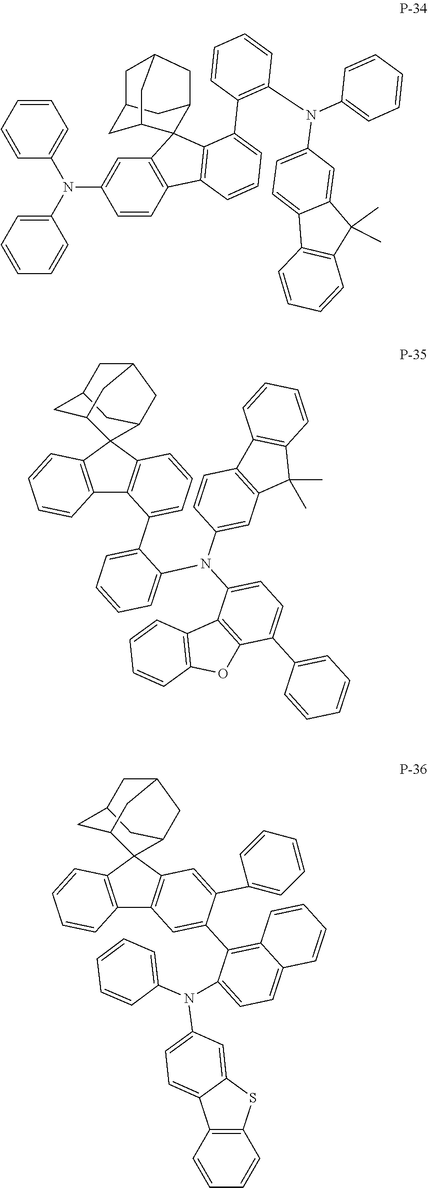 [SMILES: CC1(C)c2ccccc2-c2ccc(N(c3ccccc3)c3ccccc3-c3cccc4c3C3(c5cc(N(c6ccccc6)c6ccccc6)ccc5-4)C4CC5CC(C4)CC3C5)cc21 ; CC1(C)c2ccccc2-c2ccc(N(c3ccccc3-c3cccc4c3-c3ccccc3C43C4CC5CC(C4)CC3C5)c3ccc(-c4ccccc4)c4oc5ccccc5c34)cc21 ; c1ccc(-c2cc3c(cc2-c2c(N(c4ccccc4)c4ccc5c(c4)sc4ccccc45)ccc4ccccc24)-c2ccccc2C32C3CC4CC(C3)CC2C4)cc1]

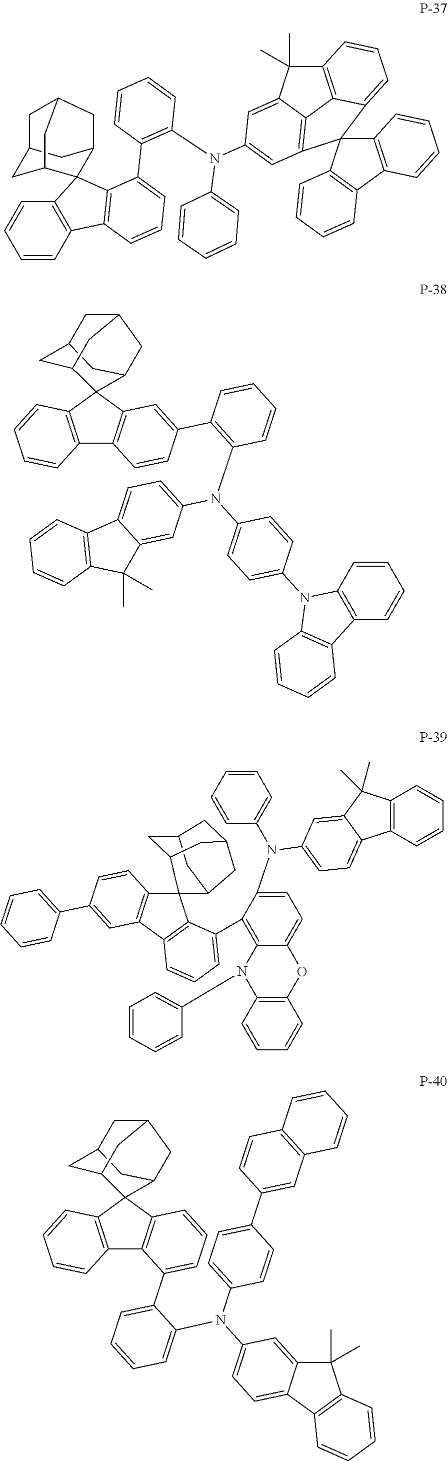 [SMILES: CC1(C)c2cccc3c2-c2c1cc(N(c1ccccc1)c1ccccc1-c1cccc4c1C1(c5ccccc5-4)C4CC5CC(C4)CC1C5)cc2C31c2ccccc2-c2ccccc21 ; CC1(C)c2ccccc2-c2ccc(N(c3ccc(-n4c5ccccc5c5ccccc54)cc3)c3ccccc3-c3ccc4c(c3)C3(c5ccccc5-4)C4CC5CC(C4)CC3C5)cc21 ; CC1(C)c2ccccc2-c2ccc(N(c3ccccc3)c3ccc4c(c3-c3cccc5c3C3(c6ccc(-c7ccccc7)cc6-5)C5CC6CC(C5)CC3C6)N(c3ccccc3)c3ccccc3O4)cc21 ; CC1(C)c2ccccc2-c2ccc(N(c3ccc(-c4ccc5ccccc5c4)cc3)c3ccccc3-c3cccc4c3-c3ccccc3C43C4CC5CC(C4)CC3C5)cc21]

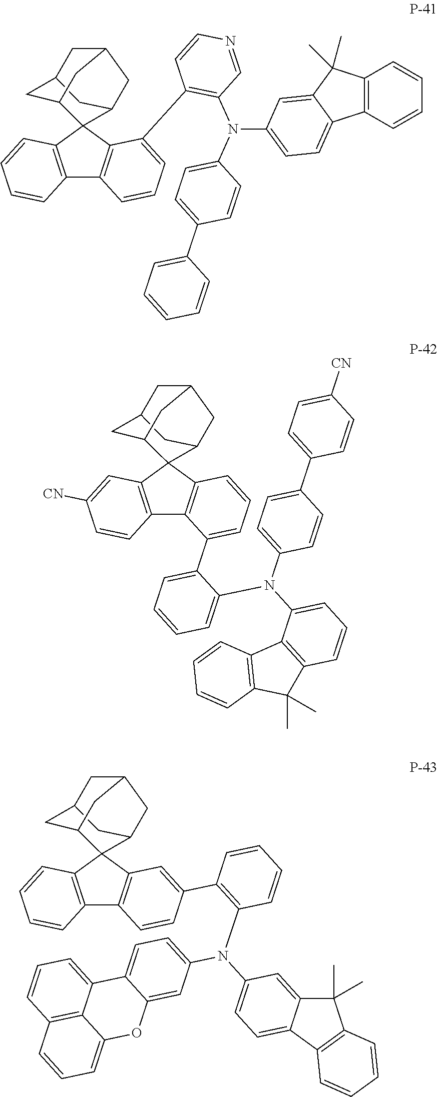

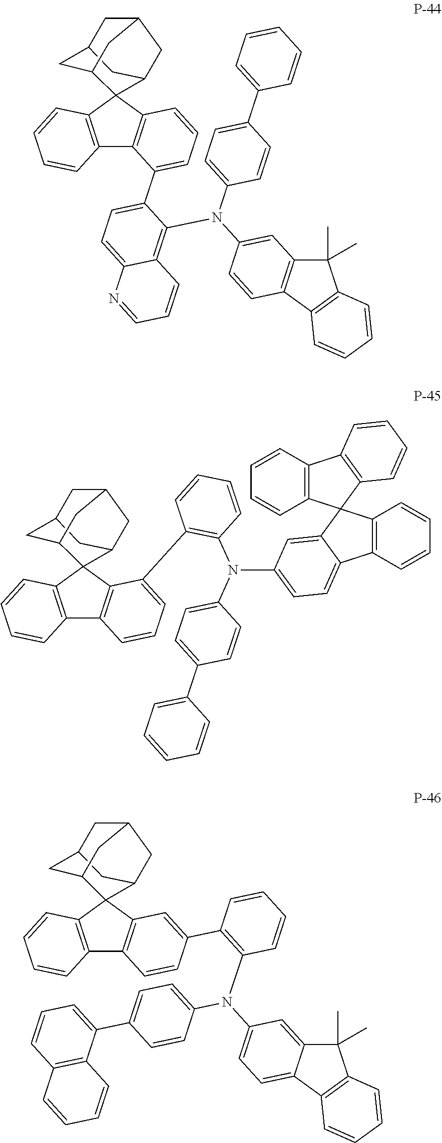 [SMILES: CC1(C)c2ccccc2-c2ccc(N(c3ccc(-c4ccccc4)cc3)c3c(-c4cccc5c4-c4ccccc4C54C5CC6CC(C5)CC4C6)ccc4ncccc34)cc21 ; c1ccc(-c2ccc(N(c3ccc4c(c3)C3(c5ccccc5-c5ccccc53)c3ccccc3-4)c3ccccc3-c3cccc4c3C3(c5ccccc5-4)C4CC5CC(C4)CC3C5)cc2)cc1 ; CC1(C)c2ccccc2-c2ccc(N(c3ccc(-c4cccc5ccccc45)cc3)c3ccccc3-c3ccc4c(c3)C3(c5ccccc5-4)C4CC5CC(C4)CC3C5)cc21]

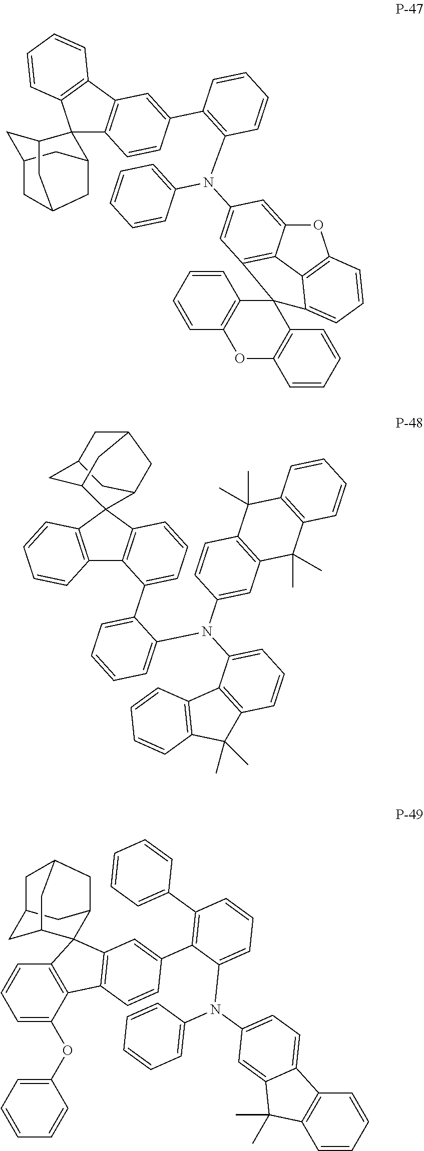 [SMILES: c1ccc(N(c2cc3c4c(c2)oc2cccc(c24)C32c3ccccc3Oc3ccccc32)c2ccccc2-c2ccc3c(c2)-c2ccccc2C32C3CC4CC(C3)CC2C4)cc1 ; CC1(C)c2ccccc2C(C)(C)c2cc(N(c3ccccc3-c3cccc4c3-c3ccccc3C43C4CC5CC(C4)CC3C5)c3cccc4c3-c3ccccc3C4(C)C)ccc21 ; CC1(C)c2ccccc2-c2ccc(N(c3ccccc3)c3cccc(-c4ccccc4)c3-c3ccc4c(c3)C3(c5cccc(Oc6ccccc6)c5-4)C4CC5CC(C4)CC3C5)cc21]

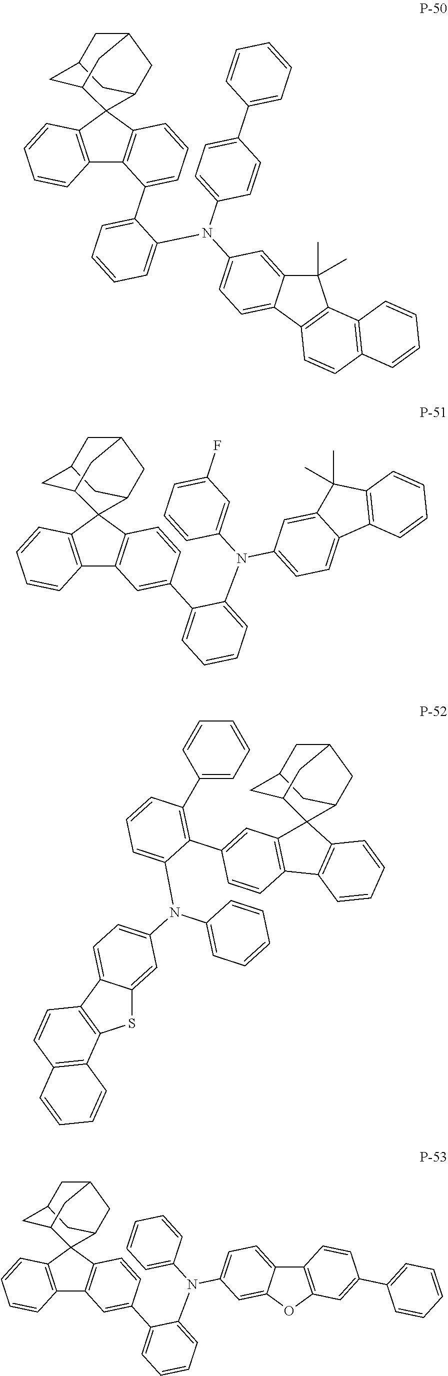 [SMILES: CC1(C)c2cc(N(c3ccc(-c4ccccc4)cc3)c3ccccc3-c3cccc4c3-c3ccccc3C43C4CC5CC(C4)CC3C5)ccc2-c2ccc3ccccc3c21 ; CC1(C)c2ccccc2-c2ccc(N(c3cccc(F)c3)c3ccccc3-c3ccc4c(c3)-c3ccccc3C43C4CC5CC(C4)CC3C5)cc21 ; c1ccc(-c2cccc(N(c3ccccc3)c3ccc4c(c3)sc3c5ccccc5ccc43)c2-c2ccc3c(c2)C2(c4ccccc4-3)C3CC4CC(C3)CC2C4)cc1 ; c1ccc(-c2ccc3c(c2)oc2cc(N(c4ccccc4)c4ccccc4-c4ccc5c(c4)-c4ccccc4C54C5CC6CC(C5)CC4C6)ccc23)cc1]

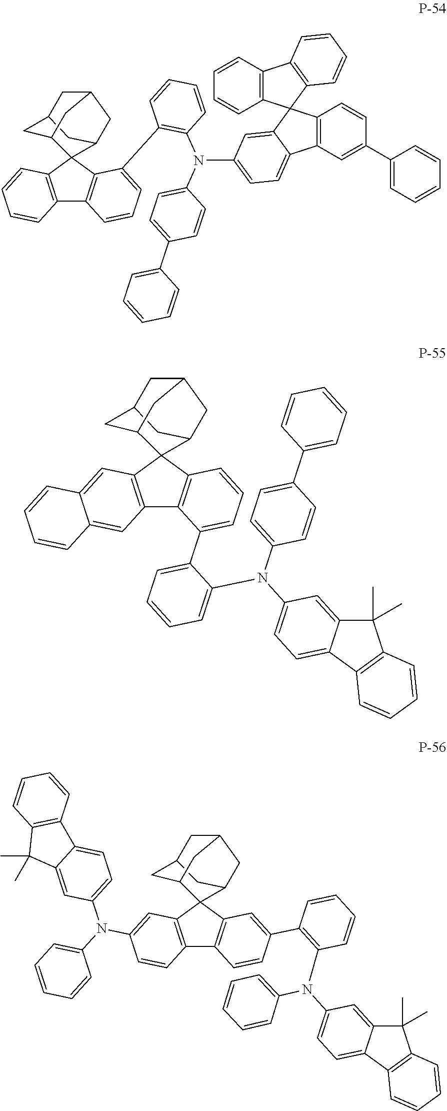 [SMILES: c1ccc(-c2ccc(N(c3ccc4c(c3)C3(c5ccccc5-c5ccccc53)c3ccc(-c5ccccc5)cc3-4)c3ccccc3-c3cccc4c3C3(c5ccccc5-4)C4CC5CC(C4)CC3C5)cc2)cc1 ; CC1(C)c2ccccc2-c2ccc(N(c3ccc(-c4ccccc4)cc3)c3ccccc3-c3cccc4c3-c3cc5ccccc5cc3C43C4CC5CC(C4)CC3C5)cc21 ; CC1(C)c2ccccc2-c2ccc(N(c3ccccc3)c3ccc4c(c3)C3(c5cc(-c6ccccc6N(c6ccccc6)c6ccc7c(c6)C(C)(C)c6ccccc6-7)ccc5-4)C4CC5CC(C4)CC3C5)cc21]

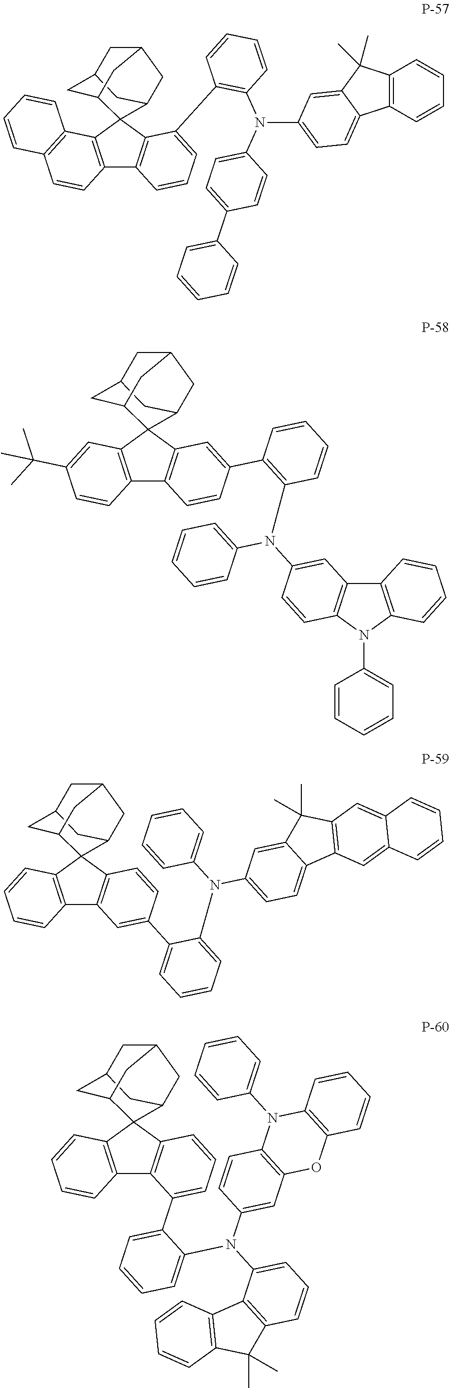 [SMILES: CC1(C)c2ccccc2-c2ccc(N(c3ccc(-c4ccccc4)cc3)c3ccccc3-c3cccc4c3C3(c5c-4ccc4ccccc54)C4CC5CC(C4)CC3C5)cc21 ; CC(C)(C)c1ccc2c(c1)C1(c3cc(-c4ccccc4N(c4ccccc4)c4ccc5c(c4)c4ccccc4n5-c4ccccc4)ccc3-2)C2CC3CC(C2)CC1C3 ; CC1(C)c2cc(N(c3ccccc3)c3ccccc3-c3ccc4c(c3)-c3ccccc3C43C4CC5CC(C4)CC3C5)ccc2-c2cc3ccccc3cc21 ; CC1(C)c2ccccc2-c2c(N(c3ccc4c(c3)Oc3ccccc3N4c3ccccc3)c3ccccc3-c3cccc4c3-c3ccccc3C43C4CC5CC(C4)CC3C5)cccc21]

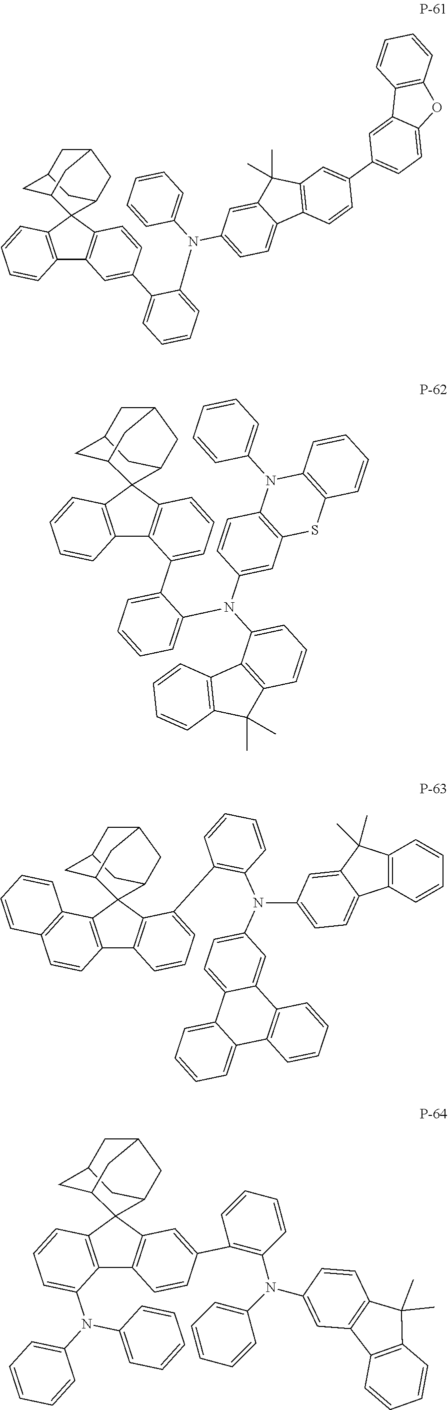 [SMILES: CC1(C)c2cc(-c3ccc4oc5ccccc5c4c3)ccc2-c2ccc(N(c3ccccc3)c3ccccc3-c3ccc4c(c3)-c3ccccc3C43C4CC5CC(C4)CC3C5)cc21 ; CC1(C)c2ccccc2-c2c(N(c3ccc4c(c3)Sc3ccccc3N4c3ccccc3)c3ccccc3-c3cccc4c3-c3ccccc3C43C4CC5CC(C4)CC3C5)cccc21 ; CC1(C)c2ccccc2-c2ccc(N(c3ccc4c5ccccc5c5ccccc5c4c3)c3ccccc3-c3cccc4c3C3(c5c-4ccc4ccccc54)C4CC5CC(C4)CC3C5)cc21 ; CC1(C)c2ccccc2-c2cc(N(c3ccccc3)c3ccccc3-c3ccc4c(c3)C3(c5cccc(N(c6ccccc6)c6ccccc6)c5-4)C4CC5CC(C4)CC3C5)ccc21]

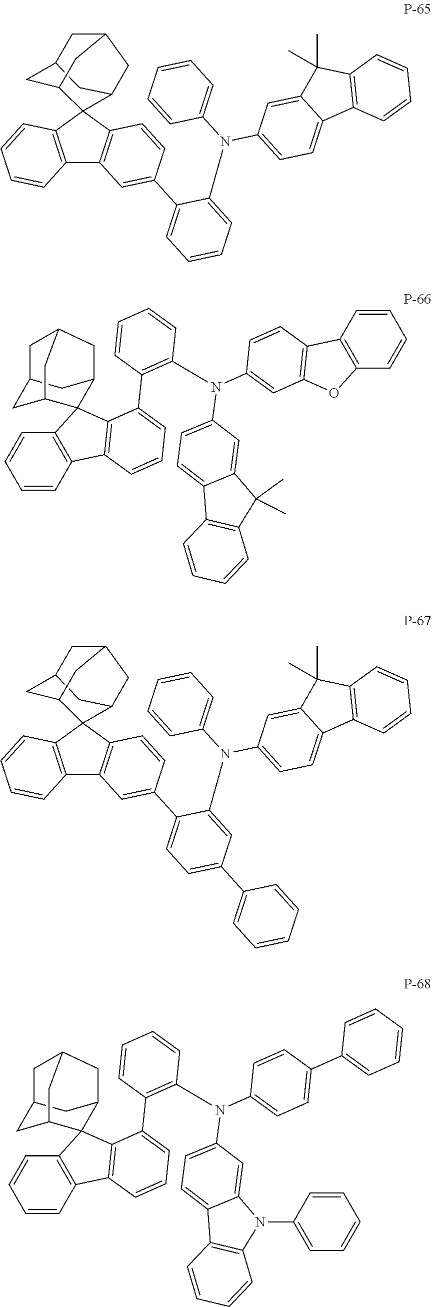 [SMILES: CC1(C)c2ccccc2-c2ccc(N(c3ccccc3)c3ccccc3-c3ccc4c(c3)-c3ccccc3C43C4CC5CC(C4)CC3C5)cc21 ; CC1(C)c2ccccc2-c2ccc(N(c3ccc4c(c3)oc3ccccc34)c3ccccc3-c3cccc4c3C3(c5ccccc5-4)C4CC5CC(C4)CC3C5)cc21 ; CC1(C)c2ccccc2-c2ccc(N(c3ccccc3)c3cc(-c4ccccc4)ccc3-c3ccc4c(c3)-c3ccccc3C43C4CC5CC(C4)CC3C5)cc21 ; c1ccc(-c2ccc(N(c3ccc4c5ccccc5n(-c5ccccc5)c4c3)c3ccccc3-c3cccc4c3C3(c5ccccc5-4)C4CC5CC(C4)CC3C5)cc2)cc1]

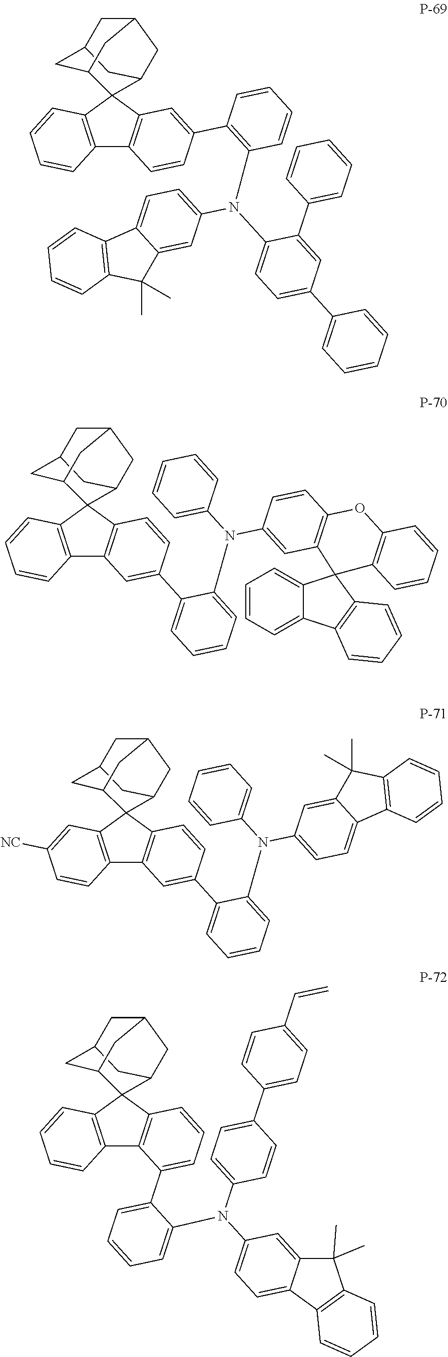 [SMILES: CC1(C)c2ccccc2-c2ccc(N(c3ccccc3-c3ccc4c(c3)C3(c5ccccc5-4)C4CC5CC(C4)CC3C5)c3ccc(-c4ccccc4)cc3-c3ccccc3)cc21 ; c1ccc(N(c2ccc3c(c2)C2(c4ccccc4O3)c3ccccc3-c3ccccc32)c2ccccc2-c2ccc3c(c2)-c2ccccc2C32C3CC4CC(C3)CC2C4)cc1 ; CC1(C)c2ccccc2-c2ccc(N(c3ccccc3)c3ccccc3-c3ccc4c(c3)-c3ccc(C#N)cc3C43C4CC5CC(C4)CC3C5)cc21 ; C=Cc1ccc(-c2ccc(N(c3ccc4c(c3)C(C)(C)c3ccccc3-4)c3ccccc3-c3cccc4c3-c3ccccc3C43C4CC5CC(C4)CC3C5)cc2)cc1]

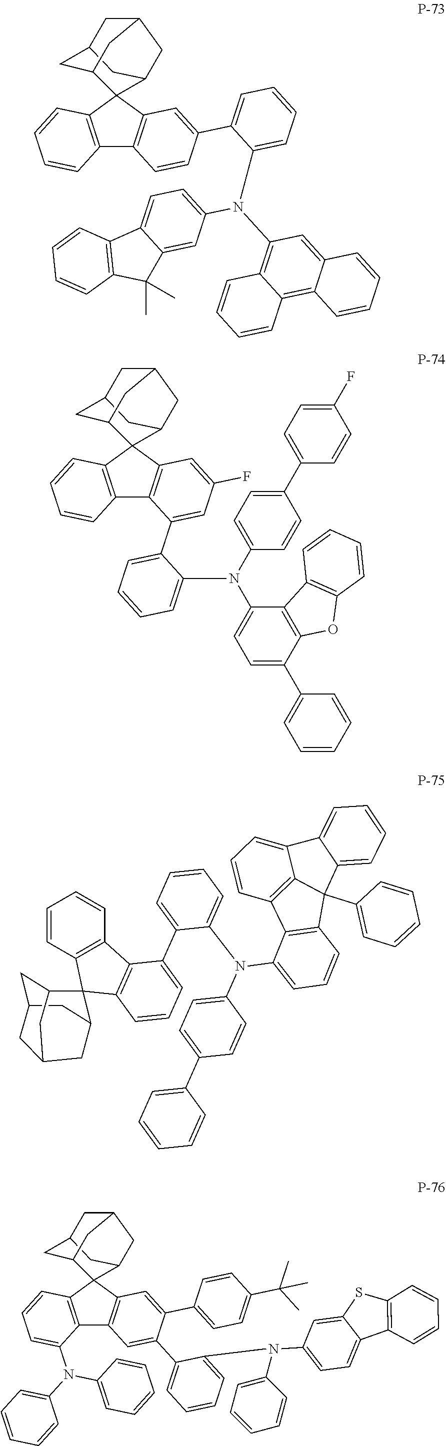 [SMILES: CC1(C)c2ccccc2-c2ccc(N(c3ccccc3-c3ccc4c(c3)C3(c5ccccc5-4)C4CC5CC(C4)CC3C5)c3cc4ccccc4c4ccccc34)cc21 ; Fc1ccc(-c2ccc(N(c3ccccc3-c3cc(F)cc4c3-c3ccccc3C43C4CC5CC(C4)CC3C5)c3ccc(-c4ccccc4)c4oc5ccccc5c34)cc2)cc1 ; c1ccc(-c2ccc(N(c3ccccc3-c3cccc4c3-c3ccccc3C43C4CC5CC(C4)CC3C5)c3cccc4c3-c3cccc5c3C4(c3ccccc3)c3ccccc3-5)cc2)cc1 ; CC(C)(C)c1ccc(-c2cc3c(cc2-c2ccccc2N(c2ccccc2)c2ccc4c(c2)sc2ccccc24)-c2c(N(c4ccccc4)c4ccccc4)cccc2C32C3CC4CC(C3)CC2C4)cc1]

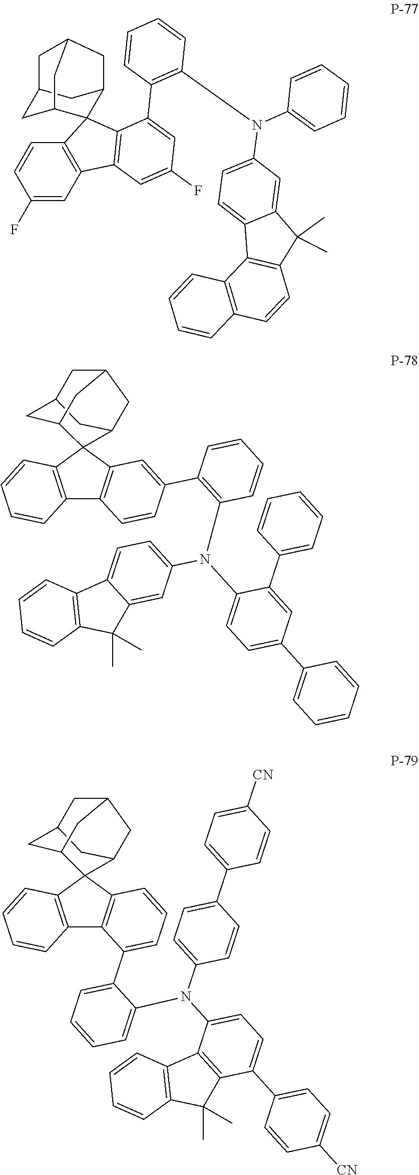 [SMILES: CC1(C)c2cc(N(c3ccccc3)c3ccccc3-c3cc(F)cc4c3C3(c5ccc(F)cc5-4)C4CC5CC(C4)CC3C5)ccc2-c2c1ccc1ccccc21 ; CC1(C)c2ccccc2-c2ccc(N(c3ccccc3-c3ccc4c(c3)C3(c5ccccc5-4)C4CC5CC(C4)CC3C5)c3ccc(-c4ccccc4)cc3-c3ccccc3)cc21 ; CC1(C)c2ccccc2-c2c(N(c3ccc(-c4ccc(C#N)cc4)cc3)c3ccccc3-c3cccc4c3-c3ccccc3C43C4CC5CC(C4)CC3C5)ccc(-c3ccc(C#N)cc3)c21]

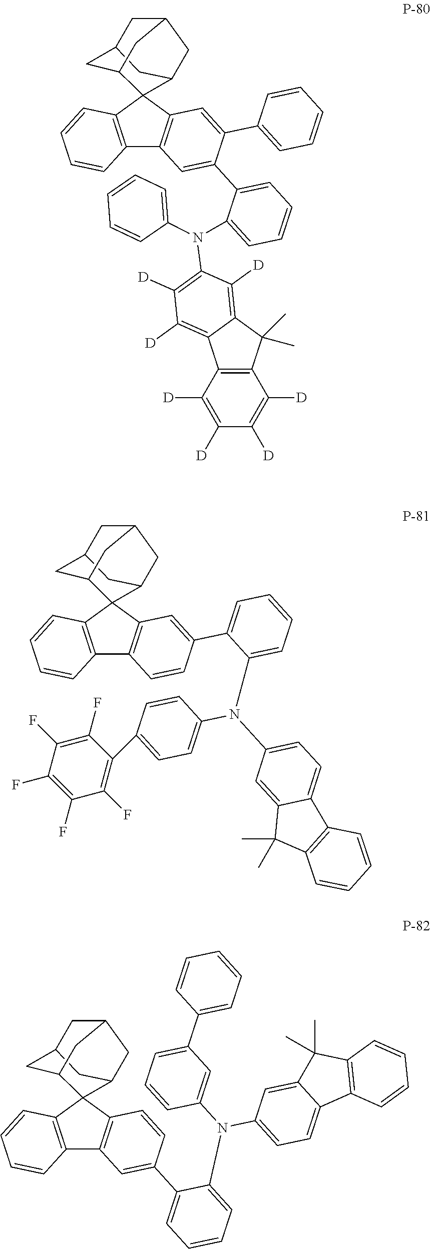 [SMILES: [2H]c1c([2H])c([2H])c2c(c1[2H])-c1c([2H])c([2H])c(N(c3ccccc3)c3ccccc3-c3cc4c(cc3-c3ccccc3)C3(c5ccccc5-4)C4CC5CC(C4)CC3C5)c([2H])c1C2(C)C ; CC1(C)c2ccccc2-c2ccc(N(c3ccc(-c4c(F)c(F)c(F)c(F)c4F)cc3)c3ccccc3-c3ccc4c(c3)C3(c5ccccc5-4)C4CC5CC(C4)CC3C5)cc21 ; CC1(C)c2ccccc2-c2ccc(N(c3cccc(-c4ccccc4)c3)c3ccccc3-c3ccc4c(c3)-c3ccccc3C43C4CC5CC(C4)CC3C5)cc21]

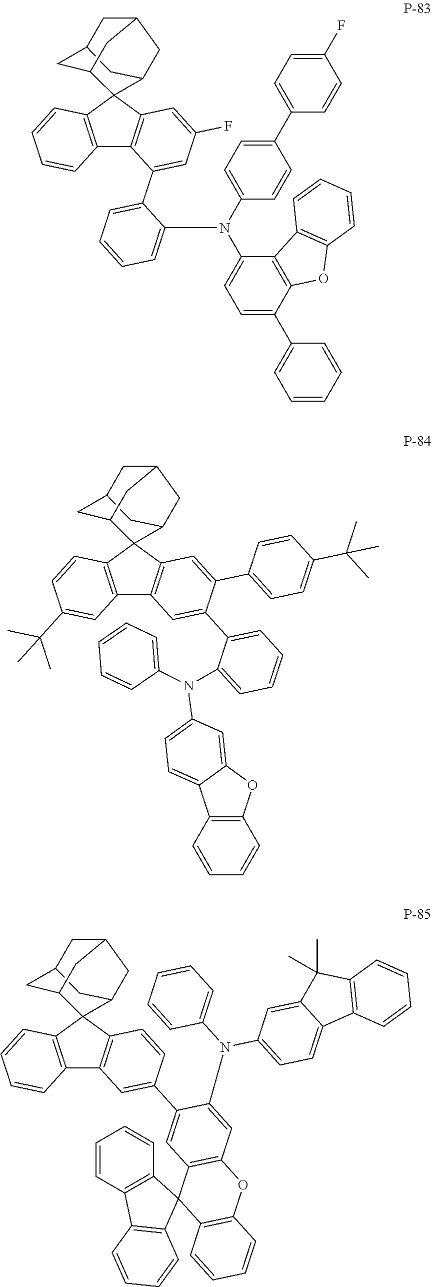 [SMILES: Fc1ccc(-c2ccc(N(c3ccccc3-c3cc(F)cc4c3-c3ccccc3C43C4CC5CC(C4)CC3C5)c3ccc(-c4ccccc4)c4oc5ccccc5c34)cc2)cc1 ; CC(C)(C)c1ccc(-c2cc3c(cc2-c2ccccc2N(c2ccccc2)c2ccc4c(c2)oc2ccccc24)-c2cc(C(C)(C)C)ccc2C32C3CC4CC(C3)CC2C4)cc1 ; CC1(C)c2ccccc2-c2ccc(N(c3ccccc3)c3cc4c(cc3-c3ccc5c(c3)-c3ccccc3C53C5CC6CC(C5)CC3C6)C3(c5ccccc5O4)c4ccccc4-c4ccccc43)cc21]

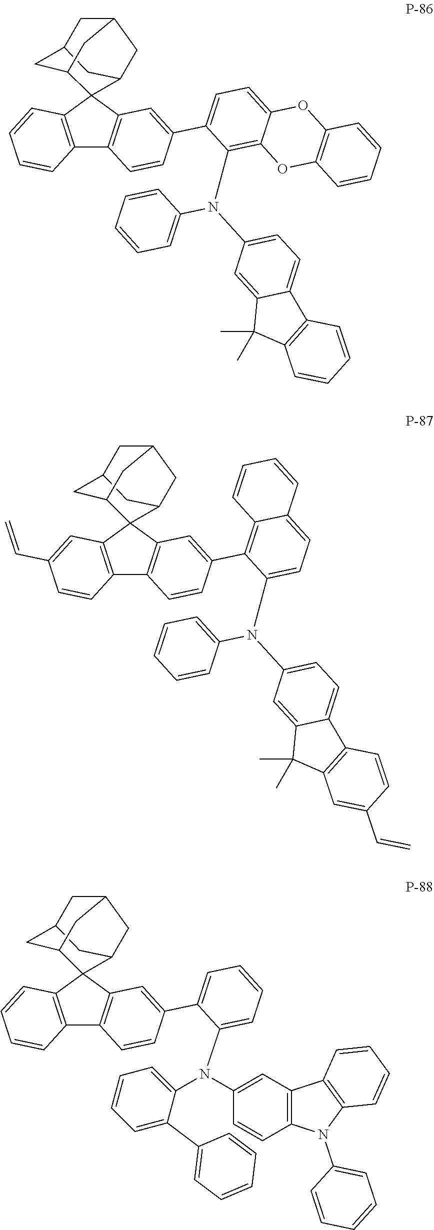 [SMILES: CC1(C)c2ccccc2-c2ccc(N(c3ccccc3)c3c(-c4ccc5c(c4)C4(c6ccccc6-5)C5CC6CC(C5)CC4C6)ccc4c3Oc3ccccc3O4)cc21 ; C=Cc1ccc2c(c1)C(C)(C)c1cc(N(c3ccccc3)c3ccc4ccccc4c3-c3ccc4c(c3)C3(c5cc(C=C)ccc5-4)C4CC5CC(C4)CC3C5)ccc1-2 ; c1ccc(-c2ccccc2N(c2ccc3c(c2)c2ccccc2n3-c2ccccc2)c2ccccc2-c2ccc3c(c2)C2(c4ccccc4-3)C3CC4CC(C3)CC2C4)cc1]

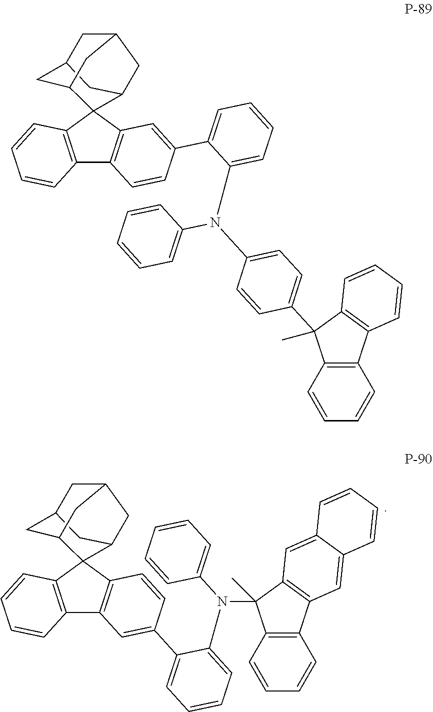 [SMILES: CC1(c2ccc(N(c3ccccc3)c3ccccc3-c3ccc4c(c3)C3(c5ccccc5-4)C4CC5CC(C4)CC3C5)cc2)c2ccccc2-c2ccccc21 ; CC1(N(c2ccccc2)c2ccccc2-c2ccc3c(c2)-c2ccccc2C32C3CC4CC(C3)CC2C4)c2ccccc2-c2cc3ccccc3cc21]

7. An organic electric device comprising:
a first electrode;
a second electrode; and
an organic material layer formed between the first electrode and the second electrode,
wherein the organic material layer contains the compounds represented by Chemical Formula 1 of claim 1 alone or in mixtures thereof.

8. An organic electric device comprising:
a first electrode;
a second electrode;
an organic material layer formed between the first electrode and the second electrode; and
a capping layer,
wherein the capping layer is formed on one surface of both surfaces of the first electrode and the second electrode that is not in contact with the organic material layer, and the organic material layer or the capping layer contains the compounds represented by Chemical Formula 1 of claim 1 alone or in mixtures thereof.

9. The organic electric device of claim 7, wherein the organic material layer includes at least one of a hole injection layer, a hole transport layer, a light emitting auxiliary layer, a light emitting layer, an electron transport auxiliary layer, an electron transport layer, and an electron injection layer.

10. The organic electric device of claim 9, wherein the organic material layer includes at least one of the hole transport layer, the light emitting auxiliary layer, and the light emitting layer.

11. The organic electric device of claim 7, wherein the organic material layer contains two or more stacks including a hole transport layer, a light emitting layer, and an electron transport layer sequentially formed on the anode.

12. The organic electric device of claim 11, wherein the organic material layer further includes a charge generation layer formed between the two or more stacks.

13. An electronic device comprising:
a display device including the organic electric device of claim 7; and
a controller for driving the display device.

14. The electronic device of claim 13, wherein the organic electric device is selected from the group consisting of an organic electroluminescent device, an organic solar cell, an organic photoreceptor, an organic transistor, a device for monochromatic lighting, a device for a quantum dot display, and the like.

15. The organic electric device of claim 8, wherein the organic material layer includes at least one of a hole injection layer, a hole transport layer, a light emitting auxiliary layer, a light emitting layer, an electron transport auxiliary layer, an electron transport layer, and an electron injection layer.

16. The organic electric device of claim 15, wherein the organic material layer includes at least one of the hole transport layer, the light emitting auxiliary layer, and the light emitting layer.

17. The organic electric device of claim 8, wherein the organic material layer contains two or more stacks including a hole transport layer, a light emitting layer, and an electron transport layer sequentially formed on the anode.

18. The organic electric device of claim 17, wherein the organic material layer further includes a charge generation layer formed between the two or more stacks.

19. An electronic device comprising:
a display device including the organic electric device of claim 8; and
a controller for driving the display device.

20. The electronic device of claim 19, wherein the organic electric device is selected from the group consisting of an organic electroluminescent device, an organic solar cell, an organic photoreceptor, an organic transistor, a device for monochromatic lighting, a device for a quantum dot display, and the like.
